(19) United States
(12) Patent Application Publication
(10) Pub. No.: US 2015/0291658 A1
(43) Pub. Date: Oct. 15, 2015

(54) NOVEL CHONDRAMIDE DERIVATIVES

(71) Applicant: HELMOLTZ-ZENTRUM FÜR INFEKTIONSFORSCHUNG, Braunschweig (DE)

(72) Inventors: Rolf Müller, Blieskastel (DE); Stephan Hüttel, Saarbrücken (DE); Jennifer Herrmann, Saarbrücken (DE); Uli Kazmaier, Homburg (DE); Dominic Becker, Saarbrücken (DE)

(21) Appl. No.: 14/646,824

(22) PCT Filed: Nov. 22, 2013
(86) PCT No.: PCT/EP2013/003540

§ 371 Date: May 22, 2015

(30) Foreign Application Priority Data
Nov. 23, 2012 (EP) 12007921.5

(51) Int. Cl. C07K 5/083; A61K 38/06

(52) U.S. Cl. CPC C07K 5/0806; A61K 38/06

(57) ABSTRACT

The present invention provides novel chondramide derivatives of formula (I) which can be used for the treatment of cancer.

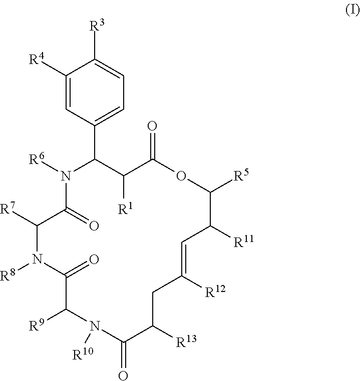

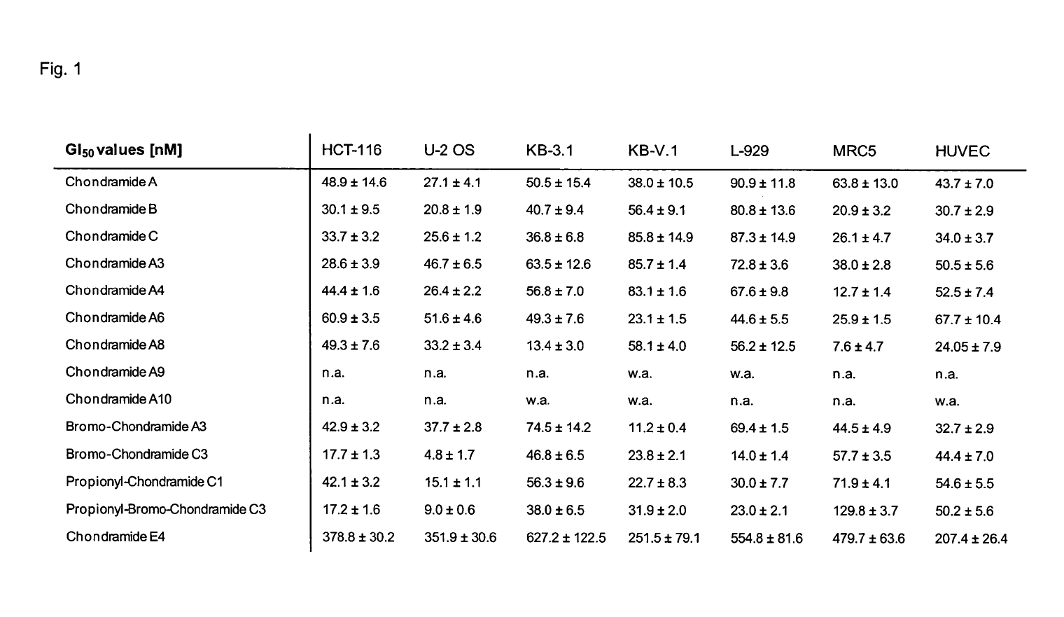

Chondramides A to D are cyclodepsipeptides that were originally isolated from terrestrial strains of Chondromyces crocatus (Kunze, B., Jansen, R., Sasse, F., Höfle, G., and Reichenbach, H. (1995). Chondramides A˜D, New Antifungal and Cytostatic Depsipeptides from Chondromyces crocatus (Myxobacteria) Production, Physico-chemical and Biological Properties. J. Antibiot. 48, 1262-1266).

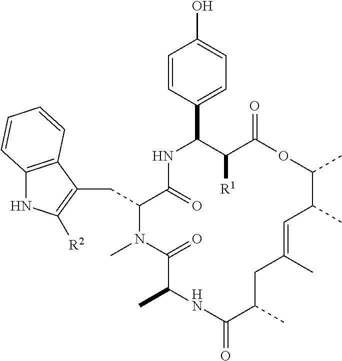

| | | |
|---|---|---|
| Chondramide A: | R¹ = OMe; | R² = H |
| Chondramide B: | R¹ = OMe; | R² = Cl |
| Chondramide C: | R¹ = H; | R² = H |
| Chondramide D: | R¹ = H; | R² = Cl |

Chondramides are structurally very similar to jasplakinolide/jaspamide isolated from marine sponges and like those act on actin, one of the major components of the eukaryotic cytoskeleton. Both, jasplakinolide and chondramide, are able to induce G-actin polymerization in vitro under non-polymerizing (low salt) conditions.

The cytoskeletal proteins actin and tubulin are involved in signaling and are critical for cell motility, maintenance of cell shape, transport mechanisms, cytokinesis, and mitosis. In particular, antimitotic agents directly targeting microtubules are well established chemotherapeutics for the treatment of cancer. However, no actin-targeting drug is currently in clinical use, although both, tubulin and actin, have been thought to be valid cancer targets. For the last years, migration of cancer cells has become an extensively studied target in cancer therapy, which might also be inhibited by actin-targeting cytostatic drugs. Despite being used as therapeutic lead, labeled chondramides are therefore also of interest as molecular probes to study actin dynamics.

More than ten years after the discovery of the 18-membered macrocyclic chondramides, two independent reports on the total synthesis of chondramide C, enabling the configurational assignment of all stereogenic centers, were published (Eggert, U., Diestel, R., Sasse, F., Jansen, R., Kunze, B., and Kalesse, M. (2008). Chondramide C: Synthesis, Configurational Assignment, and Structure-Activity Relationship Studies. Angew. Chem. Int. Ed. 47, 6478-6482; Waldmann, H., Hu, T.-S., Renner, S., Menninger, S., Tannert, R., Oda, T., and Arndt, H.-D. (2008). Total Synthesis of Chondramide C and Its Binding Mode to F-Actin. Angew. Chem. Int. Ed. 47, 6473-6477). Further, a total synthesis of chondramide A containing 3-amino-2-methoxy-3-arylpropanoic acid instead of β-tyrosine has been published (Schmauder, A., Sibley, L. D., and Maier, M. E. (2010). Total Synthesis and Configurational Assignment of Chondramide A. Chem. Eur. J. 16, 4328-4336).

The present invention provides compounds of formula (I):

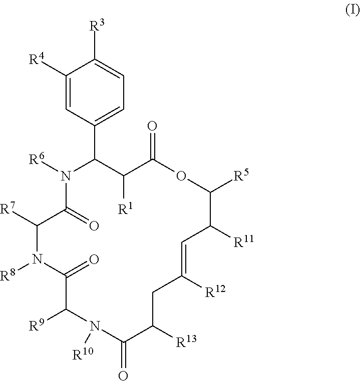

wherein
R¹ is a hydrogen atom, a hydroxy group, an alkyl, an alkenyl, or a heteroalkyl group;
R³ is a hydrogen atom, a halogen atom, a phosphate group, a hydroxy group, an amino group, a thiol group, an alkyl, an alkenyl, an alkynyl, a heteroalkyl, a cycloalkyl, a heterocycloalkyl, an alkylcycloalkyl, a heteroalkylcycloalkyl, an aryl, a heteroaryl, an aralkyl or a heteroaralkyl group;
R⁴ is a halogen atom or a hydrogen atom;
R⁵ is a hydrogen atom or an alkyl, an alkenyl or a heteroalkyl group;
R⁶ is a hydrogen atom or an alkyl group;
R⁷ is a hydrogen atom, an alkyl, an alkenyl, an alkynyl, a heteroalkyl, a cycloalkyl, a heterocycloalkyl, an alkylcycloalkyl, a heteroalkylcycloalkyl, an aryl, a heteroaryl, an aralkyl or a heteroaralkyl group;
R⁸ is a hydrogen atom or an alkyl group;
R⁹ is a hydrogen atom, an alkyl, an alkenyl, an alkynyl, a heteroalkyl, a cycloalkyl, a heterocycloalkyl, an alkylcycloalkyl, a heteroalkylcycloalkyl, an aryl, a heteroaryl, an aralkyl or a heteroaralkyl group;
R¹⁰ is a hydrogen atom or an alkyl group;
R¹¹ is a hydrogen atom or an alkyl group;
R¹² is a hydrogen atom or an alkyl group;
R¹³ is a hydrogen atom or an alkyl group; and/or
R⁷ and R⁸ and/or R⁹ and R¹⁰ together are part of an optionally substituted heterocycloalkyl group;
or a pharmaceutically acceptable salt, solvate or hydrate or a pharmaceutically acceptable formulation thereof.

The expression alkyl refers to a saturated, straight-chain or branched hydrocarbon group that contains from 1 to 20 carbon atoms, preferably from 1 to 12 carbon atoms, especially from 1 to 6 (e.g. 1, 2, 3 or 4) carbon atoms, for example a methyl, ethyl, propyl, iso-propyl, n-butyl, iso-butyl, sec-butyl, tert-butyl, n-pentyl, iso-pentyl, n-hexyl, 2,2-dimethylbutyl or n-octyl group.

The expressions alkenyl and alkynyl refer to at least partially unsaturated, straight-chain or branched hydrocarbon groups that contain from 2 to 20 carbon atoms, preferably from 2 to 12 carbon atoms, especially from 2 to 6 (e.g. 2, 3 or 4) carbon atoms, for example an ethenyl (vinyl), propenyl (allyl), iso-propenyl, butenyl, ethinyl, propinyl, butinyl, acetylenyl, propargyl, isoprenyl or hex-2-enyl group. Preferably, alkenyl groups have one or two (especially preferably one) double bond(s), and alkynyl groups have one or two (especially preferably one) triple bond(s).

Furthermore, the terms alkyl, alkenyl and alkynyl refer to groups in which one or more hydrogen atoms have been replaced by a halogen atom (preferably F or Cl) such as, for example, a 2,2,2-trichloroethyl or a trifluoromethyl group.

The expression heteroalkyl refers to an alkyl, alkenyl or alkynyl group in which one or more (preferably 1, 2 or 3) carbon atoms have been replaced by an oxygen, nitrogen, phosphorus, boron, selenium, silicon or sulfur atom (preferably by an oxygen, sulfur or nitrogen atom) or by a SO or a SO₂ group. The expression heteroalkyl furthermore refers to a carboxylic acid or to a group derived from a carboxylic acid, such as, for example, acyl, acylalkyl, alkoxycarbonyl, acyloxy, acyloxyalkyl, carboxyalkylamide or alkoxycarbonyloxy.

Preferably, a heteroalkyl group contains from 1 to 12 carbon atoms and from 1 to 4 hetero atoms selected from oxygen, nitrogen and sulphur (especially oxygen and nitrogen). Especially preferably, a heteroalkyl group contains from 1 to 6 (e.g. 1, 2, 3 or 4) carbon atoms and 1, 2 or 3 (especially 1 or 2) hetero atoms selected from oxygen, nitrogen and sulphur (especially oxygen and nitrogen). The term C₁-C₆ heteroalkyl refers to a heteroalkyl group containing from 1 to 6 carbon atoms and 1, 2 or 3 heteroatoms selected from O, S and/or N (especially O and/or N). The term C₁-C₄ heteroalkyl refers to a heteroalkyl group containing from 1 to 4 carbon atoms and 1, 2 or 3 heteroatoms selected from O, S and/or N (especially O and/or N). Furthermore, the term heteroalkyl refers to groups in which one or more hydrogen atoms have been replaced by a halogen atom (preferably F or Cl).

Examples of heteroalkyl groups are groups of formulae: R^{a}—O—Y^{a}—, R^{a}—S—Y^{a}—, R^{a}—SO—Y^{a}—, R^{a}—SO₂—Y^{a}—, R^{a}—N(R^{b})—Y^{a}—, R^{a}—CO—Y^{a}—, R^{a}—CO—N(R^{b})—Y^{a}—, R^{a}—N(R^{b})—CO—Y^{a}—, R^{a}—O—CO—N(R^{b})—Y^{a}—, R^{a}—N(R^{b})—CO—O—Y^{a}—, R^{a}—N(R^{b})—CO—N(R^{b})—Y^{a}—, R^{a}—O—CO—O—Y^{a}—, R^{a}—N(R^{b})—C(═NR^{d})—N(R^{b})—Y^{a}—, R^{a}—CS—Y^{a}—, R^{a}—O—CS—Y^{a}—, R^{a}—CS—N(R^{b})—Y^{a}—, R^{a}—N(R^{b})—CS—Y^{a}—, R^{a}—O—CS—N(R^{b})—Y^{a}—, R^{a}—N(R^{b})—CS—O—Y^{a}—, R^{a}—N(R^{b})—CS—N(R^{c})—Y^{a}—, R^{a}—O—CS—O—Y^{a}—, R^{a}—S—CO—Y^{a}—, R^{a}—CO—S—Y^{a}—, R^{a}—S—CO—N(R^{b})—Y^{a}—, R^{a}—N(R^{b})—CO—S—Y^{a}—, R^{a}—S—CO—S—Y^{a}—, R^{a}—S—CS—Y^{a}—, R^{a}—CS—S—Y^{a}—, R^{a}—S—CS—N(R^{b})—Y^{a}—, R^{a}—N(R^{b})—CS—S—Y^{a}—, R^{a}—S—CS—O—Y^{a}—, R^{a}—O—CS—S—Y^{a}—, wherein R^{a} being a hydrogen atom, a C₁-C₆ alkyl, a C₂-C₆ alkenyl or a C₂-C₆ alkynyl group; R^{b} being a hydrogen atom, a C₁-C₆ alkyl, a C₂-C₆ alkenyl or a C₂-C₆ alkynyl group; R^{c} being a hydrogen atom, a C₁-C₆ alkyl, a C₂-C₆ alkenyl or a C₂-C₆ alkynyl group; R^{d} being a hydrogen atom, a C₁-C₆ alkyl, a C₂-C₆ alkenyl or a C₂-C₆ alkynyl group and Y^{a} being a bond, a C₁-C₆ alkylene, a C₂-C₆ alkenylene or a C₂-C₆ alkynylene group, wherein each heteroalkyl group contains at least one carbon atom and one or more hydrogen atoms may be replaced by fluorine or chlorine atoms.

Specific examples of heteroalkyl groups are methoxy, trifluoromethoxy, ethoxy, n-propyloxy, isopropyloxy, butoxy, tert-butyloxy, methoxymethyl, ethoxymethyl, —CH₂CH₂OH, —CH₂OH, —SO₂Me, methoxyethyl, 1-methoxyethyl, 1-ethoxyethyl, 2-methoxyethyl or 2-ethoxyethyl, methylamino, ethylamino, propylamino, isopropylamino, dimethylamino, diethylamino, isopropylethylamino, methylamino methyl, ethylamino methyl, diisopropylamino ethyl, methylthio, ethylthio, isopropylthio, enol ether, dimethylamino methyl, dimethylamino ethyl, acetyl, propionyl, butyryloxy, acetyloxy, methoxycarbonyl, ethoxycarbonyl, propionyloxy, acetylamino or propionylamino, carboxymethyl, carboxyethyl or carboxypropyl, N-ethyl-N-methylcarbamoyl or N-methylcarbamoyl. Further examples of heteroalkyl groups are nitrile, isonitrile, cyanate, thiocyanate, isocyanate, isothiocyanate and alkylnitrile groups.

The expression cycloalkyl refers to a saturated or partially unsaturated (for example, a cycloalkenyl group) cyclic group that contains one or more rings (preferably 1 or 2), and contains from 3 to 14 ring carbon atoms, preferably from 3 to 10 (especially 3, 4, 5, 6 or 7) ring carbon atoms. The expression cycloalkyl refers furthermore to groups in which one or more hydrogen atoms have been replaced by fluorine, chlorine, bromine or iodine atoms or by OH, ═O, SH, ═S, NH₂, ═NH, N₃ or NO₂ groups, thus, for example, cyclic ketones such as, for example, cyclohexanone, 2-cyclohexenone or cyclopentanone. Further specific examples of cycloalkyl groups are a cyclopropyl, cyclobutyl, cyclopentyl, spiro[4,5]decanyl, norbornyl, cyclohexyl, cyclopentenyl, cyclohexadienyl, decalinyl, bicyclo[4.3.0]nonyl, tetraline, cyclopentylcyclohexyl, fluorocyclohexyl or cyclohex-2-enyl group.

The expression heterocycloalkyl refers to a cycloalkyl group as defined above in which one or more (preferably 1, 2 or 3) ring carbon atoms have been replaced by an oxygen, nitrogen, silicon, selenium, phosphorus or sulfur atom (preferably by an oxygen, sulfur or nitrogen atom) or a SO group or a SO₂ group. A heterocycloalkyl group has preferably 1 or 2 ring(s) containing from 3 to 10 (especially 3, 4, 5, 6 or 7) ring atoms (preferably secected from C, O, N and S). The expression heterocycloalkyl refers furthermore to groups that are substituted by fluorine, chlorine, bromine or iodine atoms or by OH, ═O, SH, ═S, NH₂, ═NH, N₃ or NO₂ groups. Examples are a piperidyl, prolinyl, imidazolidinyl, piperazinyl, morpholinyl, urotropinyl, pyrrolidinyl, tetrahydrothiophenyl, tetrahydropyranyl, tetrahydrofuryl or 2-pyrazolinyl group and also lactames, lactones, cyclic imides and cyclic anhydrides.

The expression alkylcycloalkyl refers to groups that contain both cycloalkyl and also alkyl, alkenyl or alkynyl groups in accordance with the above definitions, for example alkylcycloalkyl, cycloalkylalkyl, alkylcycloalkenyl, alkenylcycloalkyl and alkynylcycloalkyl groups. An alkylcycloalkyl group preferably contains a cycloalkyl group that contains one or two rings having from 3 to 10 (especially 3, 4, 5, 6 or 7) ring carbon atoms, and one or two alkyl, alkenyl or alkynyl groups (especially alkyl groups) having 1 or 2 to 6 carbon atoms.

The expression heteroalkylcycloalkyl refers to alkylcycloalkyl groups as defined above in which one or more (preferably 1, 2 or 3) carbon atoms have been replaced by an oxygen, nitrogen, silicon, selenium, phosphorus or sulfur atom (preferably by an oxygen, sulfur or nitrogen atom) or a SO group or a SO₂ group. A heteroalkylcycloalkyl group preferably contains 1 or 2 rings having from 3 to 10 (especially 3, 4, 5, 6 or 7) ring atoms, and one or two alkyl, alkenyl, alkynyl or heteroalkyl groups (especially alkyl or heteroalkyl groups) having from 1 or 2 to 6 carbon atoms. Examples of such groups are alkylheterocycloalkyl, alkylheterocycloalkenyl, alkenylheterocycloalkyl, alkynylheterocycloalkyl, heteroalkylcycloalkyl, heteroalkylheterocycloalkyl and heteroalkylheterocycloalkenyl, the cyclic groups being saturated or mono-, di- or tri-unsaturated.

The expression aryl refers to an aromatic group that contains one or more rings containing from 6 to 14 ring carbon atoms, preferably from 6 to 10 (especially 6) ring carbon atoms. The expression aryl refers furthermore to groups that are substituted by fluorine, chlorine, bromine or iodine atoms or by OH, SH, NH₂, N₃ or NO₂ groups. Examples are the phenyl, naphthyl, biphenyl, 2-fluorophenyl, anilinyl, 3-nitrophenyl or 4-hydroxyphenyl group.

The expression heteroaryl refers to an aromatic group that contains one or more rings containing from 5 to 14 ring atoms, preferably from 5 to 10 (especially 5 or 6 or 9 or 10) ring atoms, and contains one or more (preferably 1, 2, 3 or 4) oxygen, nitrogen, phosphorus or sulfur ring atoms (preferably O, S or N). The expression heteroaryl refers furthermore to groups that are substituted by fluorine, chlorine, bromine or iodine atoms or by OH, SH, N₃, NH₂ or NO₂ groups. Examples are pyridyl (e.g. 4-pyridyl), imidazolyl (e.g. 2-imidazolyl), phenylpyrrolyl (e.g. 3-phenylpyrrolyl), thiazolyl, isothiazolyl, 1,2,3-triazolyl, 1,2,4-triazolyl, oxadiazolyl, thiadiazolyl, indolyl, indazolyl, tetrazolyl, pyrazinyl, pyrimidinyl, pyridazinyl, oxazolyl, isoxazolyl, triazolyl, tetrazolyl, isoxazolyl, indazolyl, indolyl, benzimidazolyl, benzoxazolyl, benzisoxazolyl, benzthiazolyl, pyridazinyl, quinolinyl, isoquinolinyl, pyrrolyl, purinyl, carbazolyl, acridinyl, pyrimidyl, 2,3′-bifuryl, pyrazolyl (e.g. 3-pyrazolyl) and isoquinolinyl groups.

The expression aralkyl refers to groups containing both aryl and also alkyl, alkenyl, alkynyl and/or cycloalkyl groups in accordance with the above definitions, such as, for example, arylalkyl, arylalkenyl, arylalkynyl, arylcycloalkyl, arylcycloalkenyl, alkylarylcycloalkyl and alkylarylcycloalkenyl groups. Specific examples of aralkyls are toluene, xylene, mesitylene, styrene, benzyl chloride, o-fluorotoluene, 1H-indene, tetraline, dihydronaphthalene, indanone, phenylcyclopentyl, cumene, cyclohexylphenyl, fluorene and indane. An aralkyl group preferably contains one or two aromatic ring systems (1 or 2 rings) containing from 6 to 10 carbon atoms and one or two alkyl, alkenyl and/or alkynyl groups containing from 1 or 2 to 6 carbon atoms and/or a cycloalkyl group containing 5 or 6 ring carbon atoms.

The expression heteroaralkyl refers to an aralkyl group as defined above in which one or more (preferably 1, 2, 3 or 4) carbon atoms have been replaced by an oxygen, nitrogen, silicon, selenium, phosphorus, boron or sulfur atom (preferably oxygen, sulfur or nitrogen), that is to say to groups containing both aryl or heteroaryl, respectively, and also alkyl, alkenyl, alkynyl and/or heteroalkyl and/or cycloalkyl and/or heterocycloalkyl groups in accordance with the above definitions. A heteroaralkyl group preferably contains one or two aromatic ring systems (1 or 2 rings) containing from 5 or 6 to 10 ring carbon atoms and one or two alkyl, alkenyl and/or alkynyl groups containing 1 or 2 to 6 carbon atoms and/or a cycloalkyl group containing 5 or 6 ring carbon atoms, wherein 1, 2, 3 or 4 of these carbon atoms have been replaced by oxygen, sulfur or nitrogen atoms.

Examples are arylheteroalkyl, arylheterocycloalkyl, arylheterocycloalkenyl, arylalkylheterocycloalkyl, arylalkenylheterocycloalkyl, arylalkynylheterocycloalkyl, arylalkylheterocycloalkenyl, heteroarylalkyl, heteroarylalkenyl, heteroarylalkynyl, heteroarylheteroalkyl, heteroarylcycloalkyl, heteroarylcycloalkenyl, heteroarylheterocycloalkyl, heteroarylheterocycloalkenyl, heteroarylalkylcycloalkyl, heteroarylalkylheterocycloalkenyl, heteroarylheteroalkylcycloalkyl, heteroarylheteroalkylcycloalkenyl and heteroarylheteroalkylheterocycloalkyl groups, the cyclic groups being saturated or mono-, di- or tri-unsaturated. Specific examples are a tetrahydroisoquinolinyl, benzoyl, 2- or 3-ethylindolyl, 4-methylpyridino, 2-, 3- or 4-methoxyphenyl, 4-ethoxyphenyl, 2-, 3- or 4-carboxyphenylalkyl group.

As already stated above, the expressions cycloalkyl, heterocycloalkyl, alkylcycloalkyl, heteroalkylcycloalkyl, aryl, heteroaryl, aralkyl and heteroaralkyl also refer to groups that are substituted by fluorine, chlorine, bromine or iodine atoms or by OH, ═O, SH, ═S, NH₂, ═NH, N₃ or NO₂ groups.

The expression “optionally substituted” especially refers to groups that are optionally substituted by fluorine, chlorine, bromine or iodine atoms or by OH, ═O, SH, ═S, NH₂, ═NH, N₃ or NO₂ groups. This expression refers furthermore to groups that may be substituted by one, two, three or more unsubstituted C₁-C₁₀ alkyl, C₂-C₁₀ alkenyl, C₂-C₁₀ alkynyl, C₁-C₁₀ heteroalkyl, C₃-C₁₈ cycloalkyl, C₂-C₁₇ heterocycloalkyl, C₄-C₂₀ alkylcycloalkyl, C₂-C₁₉ heteroalkylcycloalkyl, C₆-C₁₈ aryl, C₁-C₁₇ heteroaryl, C₇-C₂₀ aralkyl or C₂-C₁₉ heteroaralkyl groups. This expression refers furthermore especially to groups that may be substituted by one, two, three or more unsubstituted C₁-C₆ alkyl, C₂-C₆ alkenyl, C₂-C₆ alkynyl, C₁-C₆ heteroalkyl, C₃-C₁₀ cycloalkyl, C₂-C₉ heterocycloalkyl, C₇-C₁₂ alkylcycloalkyl, C₂-C₁₁ heteroalkylcycloalkyl, C₆-C₁₀ aryl, C₁-C₉ heteroaryl, C₇-C₁₂ aralkyl or C₂-C₁₁ heteroaralkyl groups.

The term halogen preferably refers to F, Cl, Br or I.

According to a preferred embodiment, all alkyl, alkenyl, alkynyl, heteroalkyl, aryl, heteroaryl, cycloalkyl, heterocycloalkyl, alkylcycloalkyl, heteroalkylcycloalkyl, aralkyl and heteroaralkyl groups described herein may independently of each other optionally be substituted.

When an aryl, heteroaryl, cycloalkyl, alkylcycloalkyl, heteroalkylcycloalkyl, heterocycloalkyl, aralkyl or heteroaralkyl group contains more than one ring, these rings may be bonded to each other via a single or double bond or these rings may be annulated.

Owing to their substitution, compounds of formula (I) may contain one or more centers of chirality. The present invention therefore includes both all pure enantiomers and all pure diastereoisomers and also mixtures thereof in any mixing ratio. The present invention moreover also includes all cis/trans-isomers of the compounds of the general formula (I) and also mixtures thereof. The present invention moreover includes all tautomeric forms of the compounds of formula (I).

Preferred are compounds, wherein R¹ is a hydrogen atom, a hydroxy group or a methoxy group; especially a hydrogen atom.

Further preferred are compounds, wherein R³ is a hydroxy group, a phosphate group, or one of the following groups:

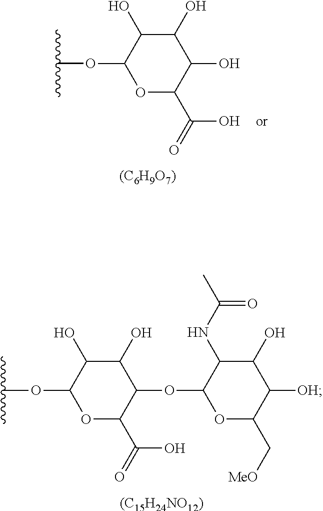

Especially preferably, R³ is a hydroxy group.

Moreover preferred are compounds, wherein R⁴ is Br, F or Cl; especially Br or Cl.

Further preferred are compounds, wherein R⁵ is a C₁-C₈ alkyl group; especially an ethyl group.

Moreover preferred are compounds, wherein R⁵ is a hydrogen atom.

Further preferred are compounds, wherein R⁵ is a is a C₂-C₈ alkyl group.

Moreover preferred are compounds, wherein R⁵ is a methyl group.

Further preferred are compounds, wherein R⁶ is a hydrogen atom or a methyl group; especially a hydrogen atom.

Further preferred are compounds, wherein R⁷ is a group of formula —CH₂-Ind, wherein Ind is an optionally substituted indole group.

Moreover preferred are compounds, wherein R⁷ is a C₁-C₆ alkyl group

Further preferred are compounds, wherein R⁷ has the following structure:

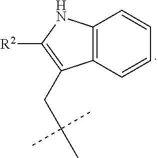

wherein R² is a hydrogen atom or Cl; especially a hydrogen atom.

Moreover preferred are compounds, wherein R⁷ has the following structure:

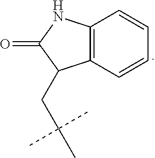

Further preferred are compounds, wherein R⁸ is a hydrogen atom or a methyl group; especially a methyl group.

Further preferred are compounds, wherein R⁹ is a methyl group.

Further preferred are compounds, wherein R¹⁰ is a hydrogen atom or a methyl group; especially a hydrogen atom.

Further preferred are compounds, wherein R¹¹ is a hydrogen atom or a methyl group; especially a methyl group.

Further preferred are compounds, wherein R¹² is a hydrogen atom or a methyl group; especially a methyl group.

Further preferred are compounds, wherein R¹³ is a hydrogen atom or a methyl group; especially a methyl group.

Further preferably, R⁷ and/or R⁹ are independently selected from H, CH₃, CH₂OH or the following groups:

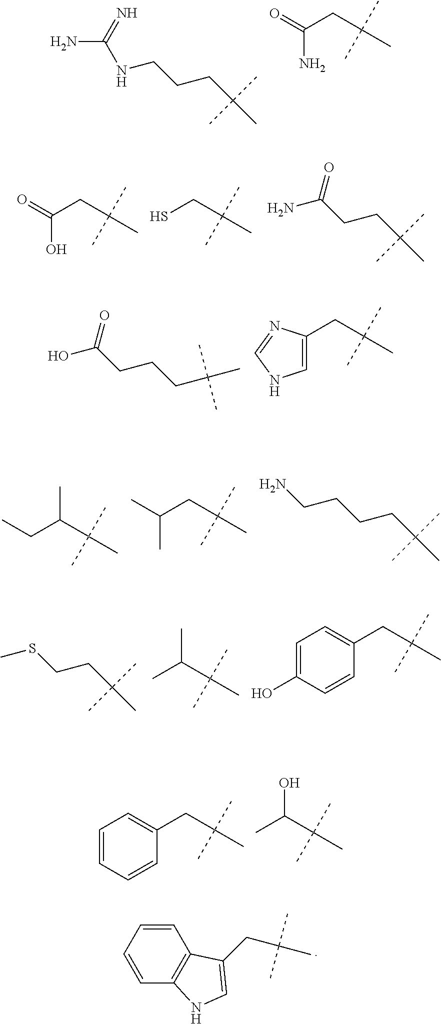

Further preferably, R⁷ and R⁸ and/or R⁹ and R¹⁰ together are a group of formula —(CH₂)₃—.

Moreover preferably, the stereochemistry of the compounds of formula (I) corresponds to the stereochemistry of known Chondramides A to D.

Especially preferred are compounds of the following structural formula (II):

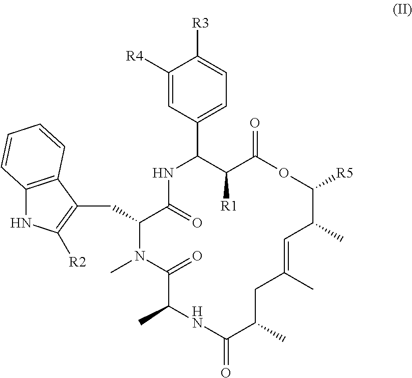

In formula (II), R¹, R², R³, R⁴ and R⁵ are as defined above.

Especially preferred are compounds of formula (II), wherein R¹ is H; R² is H; R³ is OH; R⁴ is F, Cl or Br (especially Cl or Br); and R⁵ is a C₁-C₈ alkyl group (especially an ethyl group) or a pharmaceutically acceptable salt, solvate or hydrate or a pharmaceutically acceptable formulation thereof.

Further preferred are compounds of formula (I) or (II) carrying a fluorescent group such as a group derived from: fluorescein, diacetylated fluorescein, rhodamine, Carboxytetramethylrhodamine (TAMRA), BODIPY (boron-dipyrromethene) or a cyanine (e.g. Cy5, Cy2). Such fluorescent conjugates are e.g. described in: Milroy, L.-G., Rizzo, S., Calderon, A., Ellinger, B., Erdmann, S., Mondry, J., Verveer, P., Bastiaens, P., Waldmann, H., Dehmelt, L., and Arndt, H.-D. (2012). Selective chemical imaging of static actin in live cells. J. Am. Chem. Soc. 134, 8480-8486.

Preferably, R⁷ and/or R⁹ is a group of formula —CH₂—CH₂—CH₂—CH₂—NH₂ or —CH₂—CH₂—CH₂—CH₂—NHR⁷¹, wherein R⁷¹ is fluoresceinyl, diacetylated fluoresceinyl, rhodaminyl, Carboxytetramethylrhodaminyl (TAMRA), BODIPY or a cyanine (e.g. Cy5, Cy2).

The present invention further provides pharmaceutical compositions comprising one or more compounds described herein or a pharmaceutically acceptable salt, solvate or hydrate thereof, optionally in combination with a pharmaceutically acceptable carrier.

The present invention furthermore provides compounds or pharmaceutical compositions as described herein for use in the treatment of cancer.

It is a further object of the present invention to provide a compound as described herein or a pharmaceutical composition as defined herein for the preparation of a medicament for the treatment of cancer.

The compounds of the present invention are useful in the treatment of different cancers, such as, for example, breast, colon, lung and prostate tumors, as well as bone cancer (e.g. osteosarcoma, chondrosarcoma, ESFT), leukemia (e.g. acute myeloid leukaemia, B-cell chronic lymphocytic leukaemia), sporadic endometrial cancer, melanoma, malignant melanoma, sarcoma, soft tissue sarcoma, gastric cancer, cervical cancer, hepatocellular carcinoma, pancreatic cancer, renal cancer, kidney cancer, colorectal cancer, bladder cancer, brain cancer, CNS lymphoma, esophageal cancer, NSCLC/SCLC, ovarian cancer and lymphoma (Hodgkin's disease and Non-Hodgkin's lymphoma).

Examples of pharmacologically acceptable salts of sufficiently basic compounds are salts of physiologically acceptable mineral acids like hydrochloric, hydrobromic, sulfuric and phosphoric acid; or salts of organic acids like methanesulfonic, p-toluenesulfonic, lactic, acetic, trifluoroacetic, citric, succinic, fumaric, maleic and salicylic acid. Further, a sufficiently acidic compound may form alkali or earth alkali metal salts, for example sodium, potassium, lithium, calcium or magnesium salts; ammonium salts; or organic base salts, for example methylamine, dimethylamine, trimethylamine, triethylamine, ethylenediamine, ethanolamine, choline hydroxide, meglumin, piperidine, morpholine, tris-(2-hydroxyethyl)amine, lysine or arginine salts; all of which are also further examples of salts of the compounds described herein. The compounds described herein may be solvated, especially hydrated. The hydratization/hydration may occur during the process of production or as a consequence of the hygroscopic nature of the initially water free compounds. The solvates and/or hydrates may e.g. be present in solid or liquid form.

The therapeutic use of the compounds described herein, their pharmacologically acceptable salts, solvates and hydrates, respectively, as well as formulations and pharmaceutical compositions also lie within the scope of the present invention.

The pharmaceutical compositions according to the present invention comprise at least one compound described herein and, optionally, one or more carrier substances and/or adjuvants.

As mentioned above, therapeutically useful agents that contain compounds described herein, their solvates, salts or formulations are also comprised in the scope of the present invention. In general, the compounds described herein will be administered by using the known and acceptable modes known in the art, either alone or in combination with any other therapeutic agent.

For oral administration such therapeutically useful agents can be administered by one of the following routes: oral, e.g. as tablets, dragees, coated tablets, pills, semisolids, soft or hard capsules, for example soft and hard gelatine capsules, aqueous or oily solutions, emulsions, suspensions or syrups, parenteral including intravenous, intramuscular and subcutaneous injection, e.g. as an injectable solution or suspension, rectal as suppositories, by inhalation or insufflation, e.g. as a powder formulation, as microcrystals or as a spray (e.g. liquid aerosol), transdermal, for example via an transdermal delivery system (TDS) such as a plaster containing the active ingredient or intranasal. For the production of such tablets, pills, semisolids, coated tablets, dragees and hard, e.g. gelatine, capsules the therapeutically useful product may be mixed with pharmaceutically inert, inorganic or organic excipients as are e.g. lactose, sucrose, glucose, gelatine, malt, silica gel, starch or derivatives thereof, talc, stearinic acid or their salts, dried skim milk, and the like. For the production of soft capsules one may use excipients as are e.g. vegetable, petroleum, animal or synthetic oils, wax, fat, polyols. For the production of liquid solutions, emulsions or suspensions or syrups one may use as excipients e.g. water, alcohols, aqueous saline, aqueous dextrose, polyols, glycerin, lipids, phospholipids, cyclodextrins, vegetable, petroleum, animal or synthetic oils. Especially preferred are lipids and more preferred are phospholipids (preferred of natural origin; especially preferred with a particle size between 300 to 350 nm) preferred in phosphate buffered saline (pH=7 to 8, preferred 7.4). For suppositories one may use excipients as are e.g. vegetable, petroleum, animal or synthetic oils, wax, fat and polyols. For aerosol formulations one may use compressed gases suitable for this purpose, as are e.g. oxygen, nitrogen and carbon dioxide. The pharmaceutically useful agents may also contain additives for conservation, stabilization, e.g. UV stabilizers, emulsifiers, sweetener, aromatizers, salts to change the osmotic pressure, buffers, coating additives and antioxidants.

In general, in the case of oral or parenteral administration to adult humans weighing approximately 80 kg, a daily dosage of about 1 mg to about 10,000 mg, preferably from about 5 mg to about 1,000 mg, should be appropriate, although the upper limit may be exceeded when indicated. The daily dosage can be administered as a single dose or in divided doses, or for parenteral administration, it may be given as continuous infusion or subcutaneous injection.

Especially halogenated chondramide derivatives (R⁴═F, Cl, Br) like the novel brominated chondramide derivatives (R⁴═Br) exhibit a significantly higher growth inhibitory potential on some cancer cell lines than chondramides A to D. In addition, these halogenated chondramides (like e.g. bromo-chondramides) are in average by factor 2-4 less cytotoxic for non-cancerous human cells. In vitro actin polymerization using the halogenated (e.g. brominated) chondramide derivatives showed an apparently different progression than observed for chondramides A to C, which provided evidence for a putative second target for this exciting class of natural products.

### Biological Evaluation of the Novel Chondramides

First, the compounds were tested on a small panel of cell lines derived from various origins and tissues. GI₅₀ values were compared to those determined using chondramides A˜C (see Table 1).

It has been found that the potency of chondramide E4 is approximately 10-fold reduced compared to the described chondramides, and with glycosylated derivatives (chondramide A9 and A10) activity was significantly reduced. Other derivatives exhibited a similar activity pattern as chondramides A˜C, although it has to be highlighted that halogenated (e.g. brominated) chondramide C analogs are generally more active than the reference compounds. The results from human U-2 OS osteosarcoma and HCT-116 colon carcinoma cell lines show that mean GI₅₀ values of bromo-chondramide C3 and propionyl-bromo-condramide C3 are by factor 3.5 and 2, respectively, lower than those of chondramides A˜C. With primary cells (HUVEC, human umbilical vein endothelial cells) and the fibroblast cell line MRC5 this is not the case. Whereas chondramides A˜C are comparably active on both, cancer and non-cancerous cells, brominated chondramide C3 analogs are on average by factor 2-4 less toxic for non-cancerous cells. Accordingly they show a higher selectivity.

In order to investigate whether the altered activity patterns and the reduction of activity observed with some derivatives are partly caused by hindered cellular uptake and/or adverse actin polymerization kinetics, in vitro experiments using pyrene-labeled rabbit muscle actin (Cooper, J. A., Walker, S. B., and Pollard, T. D. (1983). Pyrene actin: documentation of the validity of a sensitive assay for actin polymerization. J. Muscle Res. Cell Motil. 4, 253-262) were performed. A final concentration of 20 μM for each derivative was chosen and polymerization was followed over 85 min. Like in cell-based studies, the glycosylated chondramide A9 does not show any actin-polymerizing effect, although with the A10 analog a slow increase in G-actin polymerization can be observed, which is nevertheless significantly less pronounced than with reference compounds. The 10-fold drop in activity of chondramide E4, in terms of GI₅₀ values on cell lines, might also partly be explained by its decreased ability to polymerize actin. Chondramides A3, A6, A8, and propionyl-chondramide C1, although having GI₅₀ values comparable to chondramides A˜C, show a significantly reduced actin-polymerizing effect, merely chondramide A4 is similarly potent in the cell-free system. Halogenated (e.g. brominated) derivatives, that were found to be the most promising with respect to cancer cell growth inhibition, were only poorly active in actin polymerization studies. It is also noticeable, that bromo-chondramide A3, the least potent amongst the brominated variants, quickly polymerizes G-actin, whereas bromo-chondramide C3 analogs show significantly weaker effects.

In growth inhibition experiments it has been found that all chondramides are active in the low to mid nanomolar range, which is in accordance with previously reported values (Kunze, B., Jansen, R., Sasse, F., Höfle, G., and Reichenbach, H. (1995). Chondramides A˜D, New Antifungal and Cytostatic Depsipeptides from Chondromyces crocatus (Myxobacteria) Production, Physico-chemical and Biological Properties. J. Antibiot. 48, 1262-1266; Sasse, F., Kunze, B., Gronewold, T. M. A., and Reichenbach, H. (1998). The Chondramides: Cytostatic Agents From Myxobacteria Acting on the Actin Cytoskeleton. J. Nat. Cancer Inst. 90, 1559-1563). Reference compounds chondramides A˜D exhibited no certain specificity with respect to origin and tissue of tested cell lines; these were active in the two-digit nanomolar range on cancer cell lines, including the multidrug resistant (MDR) KB-V.1 cell line, non-cancerous cell lines, and human primary cells (HUVEC). The same applies for most novel derivatives, solely a hydroxyl group at C2′ of the polyketide segment instead of methoxy or simply hydrogen leads to an approximately 10-fold reduced potency. GI₅₀ values of glycosylated derivatives were not determinable precisely in the assay's concentration range. Most interestingly, brominated C3 analogs were clearly more active on cancer cell lines than on non-cancerous cells and aside from that generally more potent than all other tested derivatives.

For the glycosylated derivatives, halogenation on the tryptophan residue can partly restore the ability to polymerize actin when directly comparing chondramides A9 and A10. The same is observed for chondramide A3 and A4, where additional chlorination at R² increases the polymerization rate even more drastically. It has been concluded that substitution of the tryptophan residue in the peptide backbone for another amino acid lowers the molecule's affinity to actin and thereby increases availability for other putative targets. Absence of the methoxy group present in chondramide A and B further weakens this affinity. Although the actin polymerization activity is directly influenced by the propionyl or bromine group, these two substitutions nevertheless lower toxicity in absence of the methoxy group on non-cancerous cells and increase toxicity on cancer cell lines.

Novel chondramide derivatives have been prepared. Halogenated (e.g. brominated) analogs exceed, in terms of their biological activity, all chondramides described to date. Initial biological profiling of 11 new derivatives in comparison to the reference compounds (chondramides A˜C) showed that bromo-chondramide A3 and propionyl-bromo-chondramide C3 are the most active in cell-based studies, with GI₅₀ values on human cancer cell lines in the low nanomolar range. Due to productivity issues using natural sources several fully synthetic routes to these chondramide derivatives have been developed. This, in turn, allows a continuous supply of chondramide derivatives, as well as the introduction of further structural elements that are not present in the naturally produced analogues.

### EXPERIMENTAL

### General Remarks

Reactions with dry solvents were carried out in oven-dried glassware (100° C.) under nitrogen. Solvents were dried as follows: THF was distilled from LiAlH₄, CH₂Cl₂ from CaH₂, MeOH from Mg and toluene from Na. The products were purified by flash chromatography on silica gel (0.063-0.2 mm). Mixtures of EtOAc and hexanes were generally used as eluents. Analysis by TLC was carried out on commercially precoated Polygram SIL-G/UV 254 plates (Machery-Nagel, Dueren). Visualization was accomplished with UV light, KMnO₄ solution or ninhydrine. ¹H-NMR and ¹³C-NMR spectra were obtained at room temperature at 400 MHz and 100 MHz respectively. Chemical shifts are expressed in ppm relative to internal solvent. Selected signals of minor isomers are extracted from the NMR spectra of the isomeric mixtures. The enantiomeric and diastereomeric ratios were determined by HPLC using a chiral column (Reprosil 100 Chiral-NR 8 μm) or by LCMS using RP column (Luna 3μ C18, 50×4.6 mm). Optical rotation measurements were performed on a Perkin-Elmer 341 polarimeter, with concentrations given in g/100 mL. Melting points were determined with a MEL-TEMP II apparatus and are uncorrected. High-resolution mass spectra and elemental analyses were performed at Saarland Univeristy.

The synthesis of the chondramides can be subdivided into two parts: the synthesis of the peptide fragment and the synthesis of the substituted unsaturated hydroxy-carboxylic acid. Both can be synthesized independently. This allows the easy generation of a library of chondramide derivatives by combination of different peptide fragments with different substituted hydroxy acids

### Synthesis of the Peptide Fragment

The synthesis of the peptide fragment of the chondramides A-D is described in the literature. The N-terminal dipeptide fragment containing a non halogenated tryptophan can be obtained according to the following scheme:^{[3]}

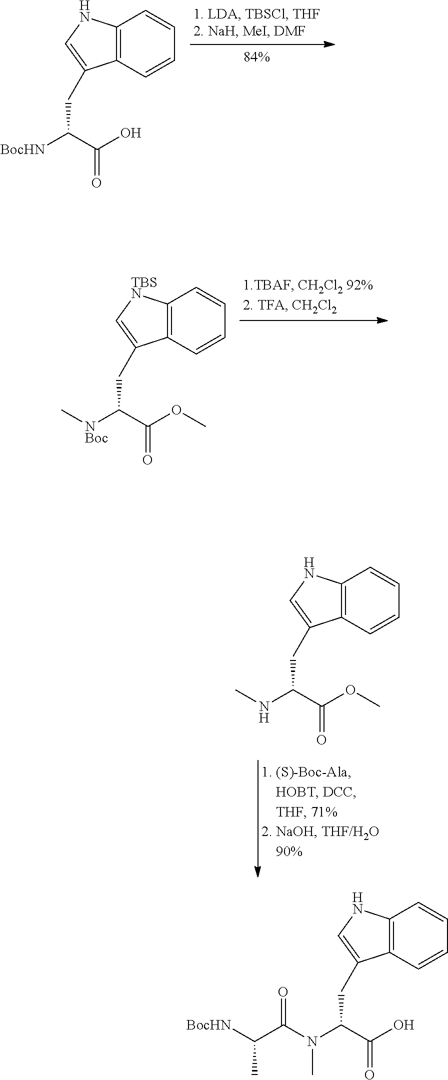

For the synthesis of the chlorinated tryptophan peptide we developed an independent route:

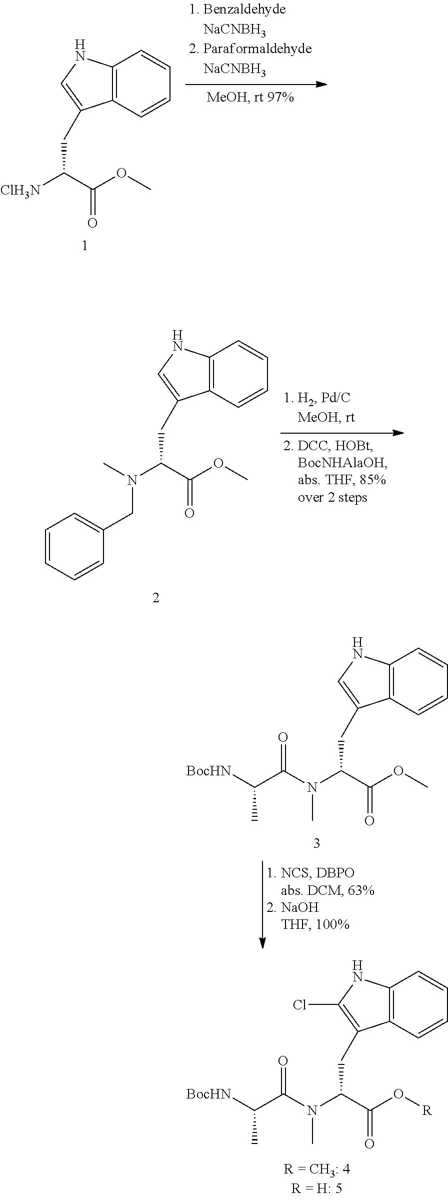

### (R)-methyl 2-(benzyl(methyl)amino)-3-(1H-indol-3-yl)propanoate (2)

5.00 g (19.6 mmol) D-Tryptophan-methylester-hydrochlorid 1 was dissolved in satd NaHCO₃ and the resulting solution was extracted with DCM (5×). The combined organic layers were dried over Na₂SO₄ and the solvent was removed under reduced pressure. To a solution of the crude product in 150 mL dry MeOH were added 1.42 g (22.6 mmol) NaCNBH₃, 2.29 g (21.6 mmol) benzaldehyde and 1.18 g (19.6 mmol) acetic acid at room temperature. After 12 h at this temperature, 1.95 g (21.6 mmol) paraformaldehyde, 1.42 g (22.6 mmol) NaCNBH₃ and 1.18 g (19.6 mmol) acetic acid were added. After further 12 h, the solvent was removed under reduced pressure, the residue was dissolved in satd NaHCO₃, the aqueous layer was extracted with DCM and the combined organic layers were dried over Na₂SO₄. Removal of the solvent under reduced pressure and purification by flash chromatography (hexanes/ethyl acetate 7:3) resulted in the isolation of 2 (6.14 g, 19.0 mmol, 97%) as white solid. R_{f}: 0.27 (hexanes/ethyl acetate 7:3). [α]²⁰_{D}=+59.7° (c=1.0, CHCl₃). ¹H NMR (400 MHz, CDCl₃): δ 2.39 (s, 3H), 3.14 (m, 1H), 3.37 (m, 1H), 3.65 (m, 1H), 3.67 (s, 3H), 3.76 (m, 1H), 3.88 (m, 1H), 7.02 (s, 1H), 7.16 (m, 1H), 7.19 (t, J=7.5 Hz, 1H), 7.31 (m, 6H), 7.51 (d, J=7.5 Hz, 1H), 7.95 (s, NH). ¹³C NMR (100 MHz, CDCl₃): δ 25.6, 38.2, 50.9, 58.8, 66.3, 111.0, 112.3, 118.6, 119.2, 121.8, 122.6, 126.9, 127.5, 128.2, 128.8, 136.1, 139.3, 172.6. HRMS (Cl) m/z calc. for C₂₀H₂₃N₂O₂ (M+H)⁺: 323.1754. found 323.1719. Anal. Calc for C₂₀H₂₂N₂O₂ (322.40): C, 74.51; H, 6.88; N, 8.69. found C, 73.79; H, 6.85; N, 8.48.

### (R)-methyl-2-((S)-2-((tert-butoxycarbonyl)amino)-N-methylpropanamido)-3-(1H-indol-3-yl) propanoate (3)

A solution of 3.75 g (11.5 mmol) 2 and 375 mg Pd/C (10% on carbon) in 40 mL dry MeOH was stirred under hydrogen atmosphere (1013 hPa) for 24 h. After filtration over celite and removal of the solvent under reduced pressure the crude product was dissolved in 100 mL dry THF. 2.16 g (11.4 mmol) N-Boc-L-Alanin, 154 mg (1.14 mmol) HOBt*aq and 2.36 g (11.4 mmol) DCC were added at 0° C. and the mixture was allowed to warm up to room temperature overnight. The precipitate was filtered off and the organic layer was washed twice with 1 M KHSO₄, H₂O, satd NaHCO₃-solution and brine, dried over Na₂SO₄ and concentrated in vacuo. Purification by flash chromatography (hexanes/ethyl acetate 1:1) provided dipeptide 3 (3.92 g, 9.72 mmol, 85%) as a white solid. R₁: 0.25 (hexanes/ethyl acetate 1:1). [α]²⁰_{D}+53.3° (c=1.0, CHCl₃). Mp: 68-69° C. 8:1 Mixture of rotamers. ¹H NMR (400 MHz, CDCl₃): δ 0.91 (d, J=6.8 Hz, 3H), 1.39 (s, 1H), 1.43 (s, 8H), 2.82 (s, 2.5H), 2.92 (s, 0.5H), 3.19 (m, 0.15H), 3.29 (dd, J=15.3, 11.6 Hz, 0.85H), 3.46 (dd, J=15.6, 4.3 Hz, 1H), 3.75 (s, 3H), 4.49 (m, 1H), 4.84 (m, 0.1H), 5.27 (dd, J=11.0, 6.0 Hz, 0.9H), 5.46 (d, J=7.8 Hz, NH), 6.99 (d, J=2.3 Hz, 1H), 7.11 (m, 1H), 7.19 (m, 1H), 7.34 (d, J=7.8 Hz, 1H), 7.58 (d, J=7.8 Hz, 1H), 8.25 (s, 1H, NH). ¹³C NMR (100 MHz, CDCl₃): δ 18.2, 24.4, 28.3, 32.8, 46.5, 52.3, 58.1, 79.5, 110.7, 111.2, 118.3, 119.5, 122.1, 122.4, 127.1, 136.1, 155.1, 171.2, 173.4.

### (R)-methyl 2-((S)-2-((tert-butoxycarbonyl)amino)-N-methylpropanamido)-3-(2-chloro-1H-indol-3-yl)propanoate (4)

To a solution of 270 mg (0.67 mmol) 3 and 11.0 mg (33.0 μmol) DBPO (75%) in 81 mL dry DCM was added 94 mg (0.70 mmol) NCS at −20° C. The resulting reaction mixture was allowed to warm up to room temperature overnight, the solvent was removed under reduced pressure and the residue was dissolved in ethyl acetate. The organic layer was further washed with 1 M HCl, H₂O, satd NaHCO₃-solution and brine, dried over Na₂SO₄ and concentrated in vacuo. Purification by flash chromatography (hexanes/ethyl acetate 7:3) afforded chlorinated dipeptide 4 (183 mg, 0.42 mmol, 63%) as a white foam. R_{f}: 0.15 (hexanes/ethyl acetate 7:3). [α]²⁰_{D}=+64.2° (c=1.0, CHCl₃). Mp: 87-89° C. 8:1 Mixture of rotamers. ¹H NMR (400 MHz, CDCl₃): δ 0.79 (d, J=6.8 Hz, 3H), 1.38 (s, 1H), 1.41 (s, 8H), 2.84 (s, 2.6H), 2.97 (s, 0.4H), 3.33 (dd, J=15.0, 10.8 Hz, 1H), 3.41 (dd, J=15.8, 5.5 Hz, 1H), 3.76 (s, 3H), 4.44 (m, 1H), 5.18 (dd, J=10.8, 5.2 Hz, 1H), 5.44 (d, J=8.0 Hz, NH), 7.09 (m, 1H), 7.15 (m, 1H), 7.23 (m, 1H), 7.48 (d, J=7.8 Hz, 1H), 8.61 (s, NH). ¹³C NMR (100 MHz, CDCl₃): δ 18.2, 23.4, 24.9 (rotamere), 25.5 (rotamere), 28.3, 29.7 (rotamere), 33.6, 33.9 (rotamere), 46.4, 49.2 (rotamere), 52.4, 57.1 (rotamere), 57.9, 79.4, 107.1, 110.5, 118.0, 120.3, 121.7, 122.5, 127.2, 134.4, 155.0, 170.9, 173.2. HRMS (Cl) m/z calc. for C₂₁H₂₉ClN₃O₅ (M+H)⁺: 438.1790. found 438.1823. Anal. Calc for C₂₁H₂₈ClN₃O₅ (437.92): C, 57.60; H, 6.44; N, 9.60. found C, 58.25; H, 6.73; N, 9.13.

### (R)-2-((S)-2-((tert-butoxycarbonyl)amino)-N-methylpropanamido)-3-(2-chloro-1H-indol-3-yl)propanoic acid (5)

To a solution of 168 mg (0.38 mmol) 4 in 4 mL MeOH was added 0.60 mL (0.60 mmol) NaOH (1M in H₂O) at 0° C. The solution was allowed to warm up to room temperature overnight, the solvent was removed and the residue was dissolved in H₂O. After acidification with 1 M KHSO₄ to pH=1-2, the aqueous layer was extracted with ethyl acetate. The combined organic layers were further washed with brine, dried over Na₂SO₄ and concentrated in vacuo to receive the chlorinated dipeptide 5 (163 mg, 0.38 mmol, 100%) as a yellow foam. R_{f}: 0.31 (hexanes/ethyl acetate 1:1+1% AcOH). [α]²⁰_{D}=+41.7° (c=1.0, MeOH). Mp: 109-111° C. ¹H NMR (400 MHz, CDCl₃): δ 0.82 (d, J=6.8 Hz, 3H), 1.41 (s, 9H), 2.85 (s, 3H), 3.41 (m, 2H), 4.49 (m, 1H), 5.11 (m, 1H), 5.59 (d, J=8.0 Hz, NH), 7.11 (m, 1H), 7.15 (m, 1H), 7.24 (m, 1H), 7.48 (d, J=7.8 Hz, 1H), 8.69 (s, NH). ¹³C NMR (100 MHz, CDCl₃): δ 17.2 (rotamere), 17.8, 23.2, 24.1 (rotamere), 28.3, 29.7 (rotamere), 34.4, 46.5, 58.9, 79.8, 107.1, 110.7, 117.9, 120.3, 121.9, 122.5, 127.2, 134.4, 155.3, 173.6, 173.9. HRMS (Cl) m/z calc. for C₂₀H₂₇ClN₃O₅ (M+H)⁺: 424.1634. found 424.1634. Anal. Calc for C₂₀H₂₆ClN₃O₅ (423.89): C, 56.67; H, 6.18; N, 9.91. found C, 56.41; H, 6.37; N, 9.02.

### Synthesis of the Substituted Hydroxy Carboxylic Acid Fragment

During the syntheses of Chondramides A and C two different synthetic routes have been developed towards this fragment.

### Route 1 (Described for R⁵═R¹¹═R¹²═R¹³=Me^{[3,4]}):

Key step of this synthesis is an allylboration of an aldehyde with a chiral borane, which generates the two stereogenic centers at R⁵ and R¹¹. While acetaldehyde (R⁵=Me) gives rise to the building block for chondramide itself, other aldehydes allow variation at position R⁵. Choosing different substituted Wittig reagents allows variation at position R¹². The Substituent R¹³ is introduced stereoselectively via asymmetric alkylation allowing the modification at this position.

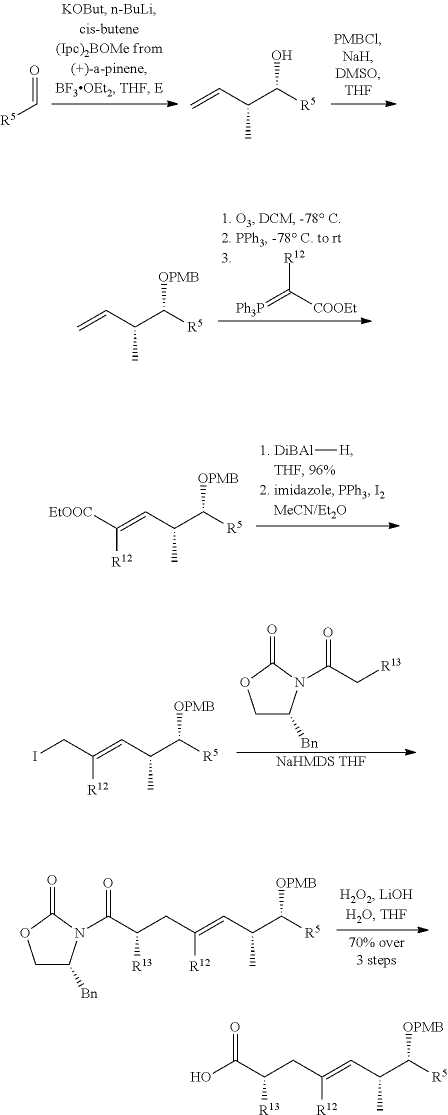

Route 2 (described for R⁵═R¹¹═R¹²═R¹³=Me^{[5]}):

Key step of this synthesis is an asymmetric dienolate aldol reaction generating the stereogenic centers at R⁵ and R¹¹ and is especially suited to vary these positions. The substituent R¹³ is introduced via asymmetric alkylation and is also variable.

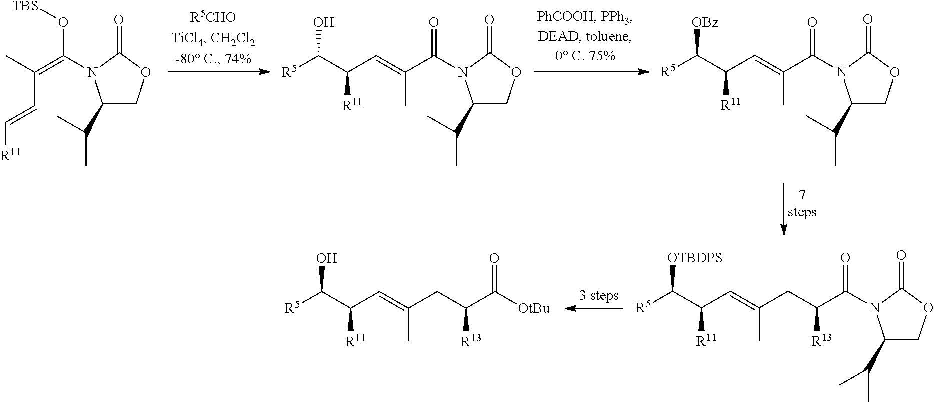

In addition to these literature known syntheses we developed an own one based on a Claisen rearrangement as a key Step. This protocol is also suitable for the synthesis of less substituted derivatives (R⁵, R¹¹, R¹², R¹³=Me, H and combinations thereof)

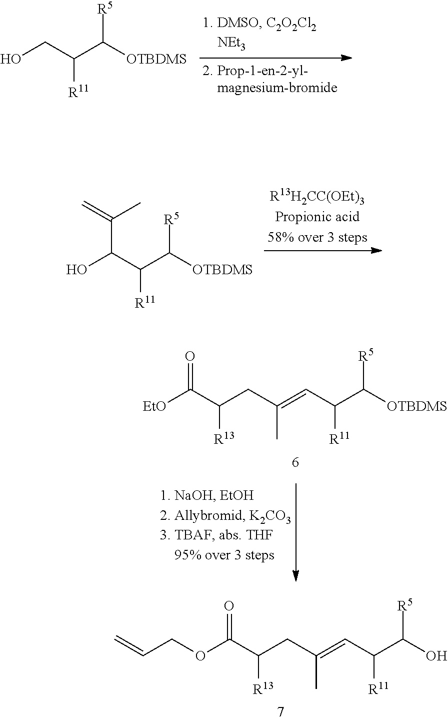

### (E)-ethyl-7-((tert-butyldimethylsilyl)oxy)-4-methylhept-4-enoate (6a)

### (R⁵═R¹¹═R¹³═H)

To a solution of 2.34 (1.58 mL, 18.4 mmol) oxalyl chloride in 47 mL dry DCM was added a solution of 2.87 g (2.61 mL, 36.7 mmol) dry DMSO in 15 mL dry DCM at −78° C. After 30 min at this temperature, 8.00 g (15.8 mmol) of 2-((tert-butyldimethylsilyl)oxy)ethanol in 40 mL dry DCM was added, after further 60 min at −78° C. 4.55 g (6.26 mL, 44.9 mmol) triethylamine was added and the reaction mixture was allowed to warm up to room temperature over 2 h. After addition of H₂O, the aqueous layer was extracted with DCM, the combined organic layers were washed with 1 M HCl, H₂O, satd NaHCO₃, brine, dried over Na₂SO₄, and concentrated to dryness to obtain the crude aldehyde.

To a solution of 44.0 mL (22.0 mmol) Iso-propenylmagnesium bromide (0.5 M in dry THF) was added the crude aldehyde at 0° C. and the reaction mixture was allowed to warm up to room temperature overnight. After hydrolysis with satd NH₄Cl-solution, the aqueous phase was extracted with diethyl ether. The combined organic layers were washed with satd NaHCO₃, H₂O and brine, and dried over Na₂SO₄. Removal of the solvent under reduced pressure afforded the crude secondary alcohol as a slight yellowish oil.

A solution of the allylic alcohol, 58.0 mg (0.79 mmol) propionic acid in 17.0 mL (15.3 g, 95.6 mmol) triethyl orthoacetate was heated to reflux overnight. Removal of the solvent by distillation under reduced pressure (40 mbar, 140° C.) and purification by flash chromatography (hexanes/ethyl acetate 95:5) resulted in the isolation of 6b (2.75 g, 9.16 mmol, 58%) as colorless oil. R_{f}: 0.38 (hexanes/ethyl acetate 95:5). ¹H NMR (400 MHz, CDCl₃): δ 0.04 (s, 6H), 0.88 (s, 9H), 1.24 (t, J=7.2 Hz, 3H), 1.62 (s, 3H), 2.21 (dt, J=7.2, 7.2 Hz, 2H), 2.30 (m, 2H), 2.38 (m, 2H), 3.56 (t, J=7.2 Hz, 2H), 4.11 (t, J=7.3 Hz, 2H), 5.15 (tq, J=7.3, 1.3 Hz, 1H). ¹³C NMR (100 MHz, CDCl₃): δ −5.3, 14.2, 16.0, 18.3, 25.9, 31.8, 33.2, 34.7, 60.2, 62.9, 121.2, 135.3, 173.4.

### (E)-allyl-7-hydroxy-4-methylhept-4-enoate (7a)

To a solution of 2.12 g (7.06 mmol) 6a in 25 mL EtOH was added 7.70 mL (7.70 mmol) NaOH (1M in H₂O) at 0° C. The solution was allowed to warm up to room temperature overnight, the solvent was removed and the residue was dissolved in H₂O. After acidification with 1 M HCl to pH=1-2, the aqueous layer was extracted with ethyl acetate. The combined organic layers were further washed with brine, dried over Na₂SO₄ and concentrated in vacuo to receive the crude unsaturated acid. To a solution of the crude acid in 25 mL dry DMF were added 1.95 g (14.1 mmol) K₂CO₃ and 0.92 mL (10.6 mmol) allyl bromide at room temperature. After 12 h at this temperature, diethyl ether was added and the organic layer was washed several times with H₂O, dried over Na₂SO₄ and concentrated under reduced pressure. The resulting crude ester and 2.03 g (7.77 mmol) TBAF trihydat was further dissolved in 25 mL dry THF. After stirring for 1 h; the reaction mixture was diluted with ethyl acetate and the organic layer was washed with 1 M HCl, brine, dried over Na₂SO₄ and concentrated in vacuo. Purification by flash chromatography (hexanes/ethyl acetate 1:1) resulted in the isolation of 7a (1.33 g, 6.68 mmol, 95%) as a colorless oil. R_{f}: 0.14 (hexanes/ethyl acetate 8:2). ¹H NMR (400 MHz, CDCl₃): δ 1.58 (s, OH), 1.66 (s, 3H), 2.27 (dt, J=6.5, 6.5 Hz, 2H), 2.35 (m, 2H), 2.46 (m, 2H), 3.60 (t, J=6.3 Hz, 2H), 4.56 (ddd, J=5.8, 1.5, 1.5 Hz, 2H), 5.15 (tq, J=7.3, 1.2 Hz, 1H), 5.23 (ddt, J=10.3, 1.3, 1.3 Hz, 1H), 5.31 (ddt, J=17.3, 1.5, 1.5 Hz, 1H), 5.89 (ddt, J=17.1, 11.3, 5.8 Hz, 1H). ¹³C NMR (100 MHz, CDCl₃): δ 16.1, 31.4, 32.9, 34.8, 62.3, 65.1, 118.2, 121.1, 132.2, 136.8, 172.9. HRMS (Cl) m/z calc. for C₁₁H₁₉O₃ (M+H)⁺: 199.1329. found 199.1346. Anal. Calc for C₁₁H₁₈O₃ (198.26): C, 66.64; H, 9.15; N−. found C, 66.27; H, 9.68; N−.

Exemplarily, also the synthesis of the ethyl substituted derivative is described:

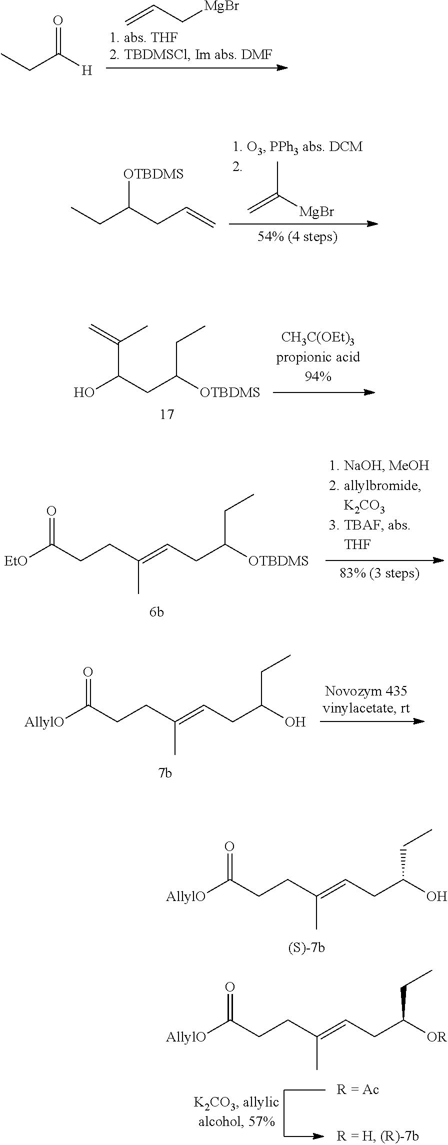

### 5-((tert-butyldimethylsilyl)oxy)-2-methylhept-1-en-3-ol (17)

To a solution of 41.3 mL (41.3 mmol, 1 M in Et₂O) allylmagnesium bromide was added 2.47 mL (34.4 mmol) propionaldehyde at 0° C. The reaction mixture was allowed to warm up to room temperature overnight, hydrolyzed with satd NH₄Cl-solution and extracted with diethyl ether. The combined organic layers were washed with brine, dried over Na₂SO₄ and concentrated by distillation.

The resulted secondary alcohol was dissolved in 80 mL abs. DMF and 5.19 g (34.4 mmol) TBDMS-Cl and 2.34 g (34.3 mmol) Imidazole were added at 0° C.: After 12 h, H₂O and diethyl ether were added and the organic layer was further washed with 1 M HCl, H₂O, satd NaHCO₃, brine, dried over Na₂SO₄ and concentrated in vacuo.

Through a solution of the protected crude product in 160 mL dry DCM was bubbled O₃ at −78° C. till a characteristic blue color appeared. 9.03 g (34.4 mmol) PPh₃ was added and further stirred for 1 h at this temperature. The reaction mixture was allowed to warm up to room temperature in 1 h and the solvent was evaporated under reduce pressure.

To the resulted crude aldehyde was added a solution of 83.0 mL (41.3 mmol, 0.5 M in THF) Iso-propenylmagnesium bromide at 0° C. The mixture was allowed to warm up to room temperature overnight, quenched with satd NH₄Cl and extracted with diethyl ether. The organic layers were washed with brine, dried over Na₂SO₄ and concentrated in vacuo. Purification by flash chromatography (hexanes/ethyl acetate 9:1) resulted in the isolation of 17 (4.78 g, 18.5 mmol, 54%) as a colorless oil. R_{f}: 0.31 (hexanes/ethyl acetate 9:1). Mixture of diastereomeres: 1:1. ¹H NMR (400 MHz, CDCl₃): δ 0.08 (s, 1.5H), 0.10 (s, 3H), 0.12 (s, 1.5H), 0.87 (m, 3H), 0.90 (s, 4.5H), 0.91 (s, 4.5H), 1.51-1.67 (m, 4H), 1.72 (s, 1.5H), 1.73 (S, 1.5H), 3.19 (d, J=2.5 Hz, 0.5 OH), 3.24 (d, J=1.25 Hz, 0.5 OH), 3.91 (m, 1H), 4.17 (m, 0.5H), 4.33 (m, 0.5H), 4.81 (m, 1H), 5.01 (m, 1H). ¹³C NMR (100 MHz, CDCl₃): δ −4.7, −4.1, 8.8, 9.9, 17.9, 18.0, 18.2, 25.8, 29.1, 30.5, 39.9, 41.0, 72.1, 72.5, 74.2, 74.9, 109.9, 110.4, 147.4, 147.8.

### (E)-ethyl 7-((tert-butyldimethylsilyl)oxy)-4-methylnon-4-enoate (6b)

A solution of 3.52 g (13.3 mmol) allylic alcohol 17, 30.0 mg (0.40 mmol) propionic acid in 14.7 mL (13.0 g, 80.0 mmol) triethyl orthoacetate was heated to reflux overnight. Removal of the solvent by distillation under reduced pressure (40 mbar, 140° C.) and purification by flash chromatography (hexanes/ethyl acetate 95:5) resulted in the isolation of 6b (4.14 g, 12.6 mmol, 94%) as colorless oil. R_{f}: 0.38 (hexanes/ethyl acetate 95:5). R_{f}: 0.33 (hexanes/ethyl acetate 95:5). ¹H NMR (400 MHz, CDCl₃): δ 0.03 (s, 6H), 0.86 (t, J=7.3 Hz, 3H), 0.88 (s, 9H), 1.25 (t, J=7.1 Hz, 3H), 1.40 (m, 2H), 1.61 (s, 3H), 2.13 (m, 2H), 2.32 (m, 2H), 2.39 (m, 2H), 3.57 (m, 1H), 4.11 (q, J=7.1 Hz, 2H), 5.18 (t, J=7.3 Hz, 1H). ¹³C NMR (100 MHz, CDCl₃): δ −4.6, −4.5, 9.7, 14.2, 16.2, 18.1, 25.9, 29.5, 33.2, 34.8, 35.5, 60.2, 73.6, 121.8, 134.6, 173.5.

### (E)-allyl 7-hydroxy-4-methylnon-4-enoate (7b)

To a solution of 4.11 g (11.8 mmol) 6b in 42 mL EtOH was added 17.6 mL (17.6 mmol) NaOH (1M in H₂O) at 0° C. The solution was allowed to warm up to room temperature overnight, the solvent was removed and the residue was dissolved in H₂O. After acidification with 1 M HCl to pH=1-2, the aqueous layer was extracted with ethyl acetate. The combined organic layers were further washed with brine, dried over Na₂SO₄ and concentrated in vacuo to receive the crude unsaturated acid. To a solution of the crude acid in 42 mL dry DMF were added 3.25 g (23.5 mmol) K₂CO₃ and 1.53 mL (17.6 mmol) allyl bromide at room temperature. After 12 h at this temperature, diethyl ether was added and the organic layer was washed several times with H₂O, dried over Na₂SO₄ and concentrated under reduced pressure. The resulting crude ester and 3.69 g (14.1 mmol) TBAF trihydat was further dissolved in 12 mL dry THF. After stirring for 12 h, the reaction mixture was diluted with ethyl acetate and the organic layer was washed with 1 M HCl, brine, dried over Na₂SO₄ and concentrated in vacuo. Purification by flash chromatography (hexanes/ethyl acetate) resulted in the isolation of 7b (2.22 g, 9.81 mmol, 83%) as a colorless oil. R_{f}: 0.24 (hexanes/ethyl acetate 8:2). ¹H NMR (400 MHz, CDCl₃): δ 0.93 (t, J=7.5 Hz, 3H), 1.46 (m, 2H), 1.64 (s, 3H), 1.65 (s, OH), 2.14 (m, 2H), 2.34 (m, 2H), 2.46 (m, 2H), 3.52 (m, 1H), 4.55 (ddd, J=5.7, 1.3, 1.3 Hz, 2H), 5.18 (m, 1H), 5.22 (ddt, J=10.5, 1.25, 1.25 Hz, 1H), 5.30 (ddt, J=17.3, 1.5, 1.5 Hz, 1H), 5.89 (ddt, J=17.0, 11.5, 5.8 Hz, 1H). ¹³C NMR (100 MHz, CDCl₃): δ 10.0, 16.1, 25.6, 29.5, 32.9, 34.8, 35.6, 65.1, 72.9, 118.2, 121.3, 132.2, 136.7, 172.9.

### (S,E)-allyl 7-hydroxy-4-methylnon-4-enoate ((S,E)-7b) and (R,E)-allyl 7-acetoxy-4-methylnon-4-enoate ((R,E)-7b-Acetate)

Entry 1: A mixture of racemic alcohol 7b (2.11 g, 9.32 mmol) in vinyl acetate (4.3 mL, 46.6 mmol) was shaken in the presence of Novozym® 435 (127 mg, 6 wt. %) for 48 h at room temperature. The enzyme was filtered off and the resulting solution was diluted with 30 mL dry diethyl ether. Purification by flash chromatography (hexanes/ethyl acetate 8:2) resulted in the isolation of (S,E)-7b (741 mg, 3.27 mmol, 35%, >99% ee) and (R,E)-7b-Acetate (1.47 g, 5.48 mmol, 59%, 60% ee) as colorless oils. The nmr data and R_{f}-value of (S,E)-7b correspond with the values of the racemic alcohol 7b. R_{f}((R,E)-7b-Acetate): 0.46 (hexanes/ethyl acetate 8:2). ¹H NMR (400 MHz, CDCl₃): δ 0.87 (t, J=7.4 Hz, 3H), 1.53 (m, 2H), 1.62 (s, 3H), 2.02 (s, 3H), 2.23 (m, 2H), 2.32 (m, 2H), 2.43 (m, 2H), 4.56 (ddd, J=5.8, 1.4, 1.4 Hz, 2H), 4.77 (m, 1H), 5.14 (tq, J=7.1, 1.9 Hz, 1H), 5.23 (ddt, J=10.4, 1.25, 1.25 Hz, 1H), 5.31 (ddt, J=17.1, 3.0, 1.5 Hz, 1H), 5.91 (ddt, J=17.2, 10.4, 5.6 Hz, 1H). ¹³C NMR (100 MHz, CDCl₃): δ 9.7, 16.1, 21.2, 26.4, 32.1, 32.9, 34.6, 65.0, 75.2, 118.1, 120.1, 132.2, 136.1, 170.8, 172.9. HPLC (OD-H, Hex:iPrOH, 5 min at 95:5, than in 5 min to 99:1, 1 mL/min): t_{R(Acetate, major)}=9.60 min. t_{R(Acetate, minor)}=7.41 min, t_{R(Alcohol, major)}=20.15 min.
Entry 2: A mixture of alcohol 7b (1.11 g, 4.66 mmol, 21% ee (R->S-enantiomere) in vinyl acetate (2.16 mL, 23.3 mmol) was shaken in the presence of Novozym® 435 (67.0 mg, 6 wt. %) for 12 h at room temperature. The enzyme was filtered off and the resulting solution was diluted with 30 mL dry diethyl ether. Purification by flash chromatography (hexanes/ethyl acetate 8:2) resulted in the isolation of (S,E)-7b (295 mg, 1.30 mmol, 28%, 39% ee) and (R,E)-7b-Acetate (829 mg, 3.09 mmol, 66%, 91% ee) as colorless oils. The nmr data and R_{f}-value of (S,E)-7b correspond with the values of the racemic alcohol 7b. R_{f} ((R,E)-7b-Acetate): 0.46 (hexanes/ethyl acetate 8:2). ¹H NMR (400 MHz, CDCl₃): δ 0.87 (t, J=7.4 Hz, 3H), 1.53 (m, 2H), 1.62 (s, 3H), 2.02 (s, 3H), 2.23 (m, 2H), 2.32 (m, 2H), 2.43 (m, 2H), 4.56 (ddd, J=5.8, 1.4, 1.4 Hz, 2H), 4.77 (m, 1H), 5.14 (tq, J=7.1, 1.9 Hz, 1H), 5.23 (ddt, J=10.4, 1.25, 1.25 Hz, 1H), 5.31 (ddt, J=17.1, 3.0, 1.5 Hz, 1H); 5.91 (ddt, J=17.2, 10.4, 5.6 Hz, 1H). ¹³C NMR (100 MHz, CDCl₃): δ 9.7, 16.1, 21.2, 26.4, 32.1, 32.9, 34.6, 65.0, 75.2, 118.1, 120.1, 132.2, 136.1, 170.8, 172.9. HPLC (OD-H, Hex:iPrOH, 5 min at 95:5, than in 5 min to 99:1, 1 mL/min): t_{R(Acetate, major)}=9.60 min, t_{R(Acetate, minor)}=7.41 min, t_{R(Alcohol, major)}=20.15 min.
A solution of (R,E)-7b-Acetate (810 mg, 3.02 mmol, 91° A) ee) and 83 mg (604 μmol) K₂CO₃ in 5 mL allylic alcohol was stirred for 12 h at 80° C. After evaporation of the solvent, 1 M KHSO₄ and DCM were added. The aqueous layer was further extracted with DCM and the combined organic layers were washed with brine, dried over Na₂SO₄ and concentrated in vacuo. Purification by flash chromatography (hexanes/ethyl acetate 8:2) afforded alcohol (R,E)-7b (386 mg, 1.71 mmol, 56%) as a colorless oil. The nmr data and R_{f}-value of (R,E)-7b correspond with the values of the racemic alcohol 7b.

### Syntheses of the β-Tyrosine-Derivative

During the syntheses of Chondramide A and D synthetic routes towards β-tyrosine^{[3,6]} and α-methoxy-β-tyrosin^{[7]} have been developed.

### β-Tyrosine:^{[3,6]}

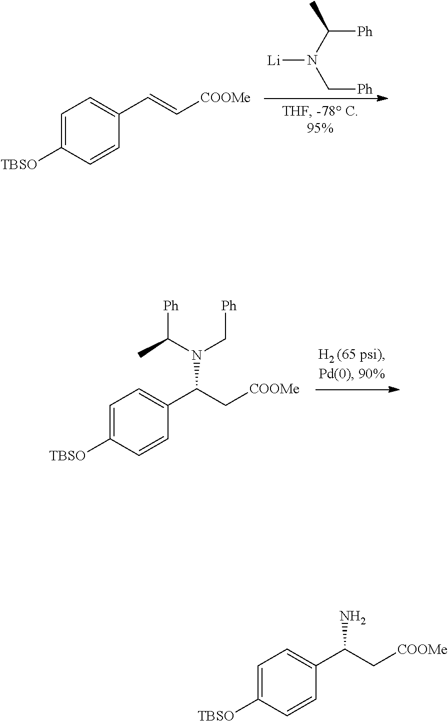

α-Methoxy-β-tyrosin^{[7]}

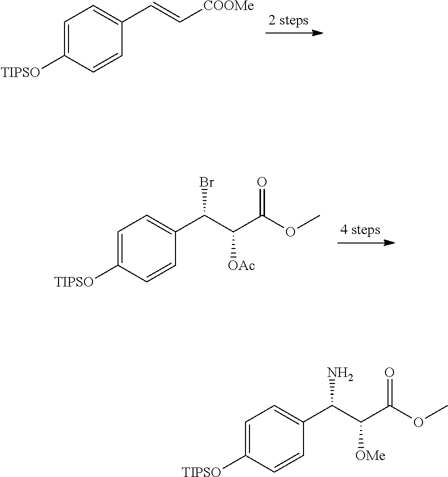

For the synthesis of halogenated β-tyrosines, we developed two independent routes giving access to chlorinated and brominated derivatives. The halogenation was carried out with O-protected hydroxyphenylglycine derivatives. Subsequent Arndt-Eistert reaction provides an active intermediate, which allows the direct coupling with the corresponding hydroxy carboxylic acid fragment.

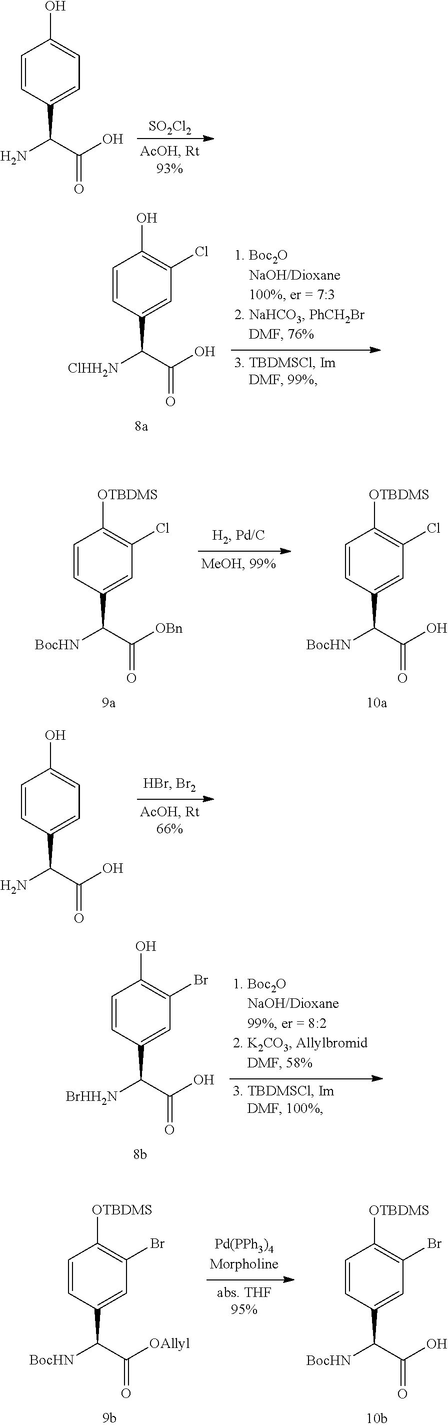

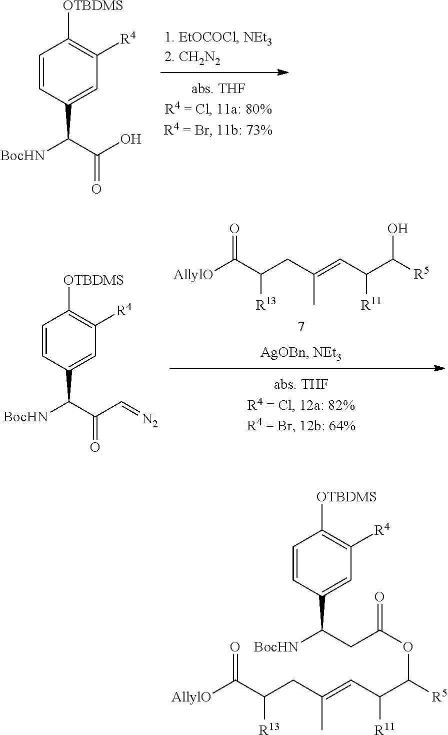

### (S)-carboxy(3-chloro-4-hydroxyphenyl)methanaminium chloride (8a)

A suspension of 4.87 g (29.1 mmol) 4-Hydroxy-L-Phenylglycine in 36 mL acetic acid was treated with 2.61 mL (32.1 mmol) sulfuryl chloride and stirred overnight at room temperature. The resulting hydrochloride was filtered off and washed several times with acetic acid and pentane. Lyophilization of the crude product resulted in the isolation of 8a (6.42 g, 27.0 mmol, 93%) as a white solid. [α]²⁰_{D}=+128.7° (c=1.0, MeOH). Mp: 191-193° C. (decomposition). ¹H NMR (400 MHz, DMSO): δ 4.77 (s, 1H), 7.07 (d, J=8.3 Hz, 1H), 7.21 (dd, J=8.4, 2.1 Hz, 1H), 7.46 (d, J=2.0 Hz, 1H), 8.79 (s, 3H). ¹³C NMR (100 MHz, DMSO): δ 55.2, 116.7, 119.6, 125.7, 127.9, 129.5, 153.8, 169.7.

### (S)-benzyl 2-((tert-butoxycarbonyl)amino)-2-(4-((tert-butyldimethylsilyl)oxy)-3-chloro-phenyl)-acetate (9a)^{[1]}

### (S)-2-((tert-butoxycarbonyl)amino)-2-(3-chloro-4-hydroxyphenyl)acetic acid 9a-I

To a solution of 8.21 g (26.9 mmol) 8a in 103 mL NaOH (1 M in H₂O) and 48 mL dioxane was added 7.04 g (32.3 mmol) Di-tert-butyl-dicarbonate at 0° C. The reaction mixture was allowed to warm up to room temperature overnight. After removal of dioxane under reduced pressure, the aqueous layer was washed with diethyl ether, acidified with 6 M HCl to pH=1-2 and extracted with ethyl acetate. The combined organic layers were washed with brine, dried over Na₂SO₄ and concentrated in vacuo to receive 8.13 g (26.9 mmol, 100%) (S)-2-((tert-butoxycarbonyl)amino)-2-(3-chloro-4-hydroxyphenyl)acetic acid 9a-I as a colorless oil. R_{f}: 0.36 (hexanes/ethyl acetate 1:1+1% AcOH). [α]²⁰_{D}=+115.1° (c=1.0, MeOH, er=7:3). Mp: 88-91° C. 7:2 Mixture of rotamers. ¹H NMR (400 MHz, CDCl₃): δ 1.26 (s, 7H), 1.43 (s, 2H), 5.04 (d, J=5.3 Hz, 0.8H), 5.25 (s, 0.2H), 5.58 (s, 0.3 NH), 6.98 (d, J=8.5 Hz, 1H), 7.19 (m, 1H), 7.37 (s, 0.3H), 7.41 (s, 0.7H), 8.11 (s, 0.7 NH). ¹³C NMR (100 MHz, CDCl₃): δ 28.0, 57.8, 82.1, 116.2, 119.9, 125.3, 127.4, 131.5, 151.1, 156.9, 173.1.

### (S)-benzyl-2-((tert-butoxycarbonyl)amino)-2-(3-chloro-4-hydroxyphenyl)acetate 9a-II

To a solution of 3.01 g (8.58 mmol) 9a-I in 40 mL dry DMF were added 721 mg (8.58 mmol) NaHCO₃ and 4.61 g (9.44 mmol) benzyl bromide at 0° C. and the reaction mixture was allowed to warm up to room temperature overnight. H₂O was added and extracted with ethyl acetate. The combined organic layers were further washed with 1 M HCl, H₂O, brine, dried over Na₂SO₄ and concentrated under reduced pressure. Purification by flash chromatography (hexanes/ethyl acetate 7:3) resulted in the isolation of (S)-benzyl-2-((tert-butoxycarbonyl)amino)-2-(3-chloro-4-hydroxyphenyl) acetate 9a-II (2.57 g, 6.55 mmol, 76%) as a colorless oil. R_{f}: 0.12 (hexanes/ethyl acetate 8:2). [α]²⁰_{D}=+10.5° (c=1.0, CHCl₃, er=7:3). ¹H NMR (400 MHz, CDCl₃): 1.43 (s, 9H), 5.16 (s, 2H), 5.28 (d, J=6.8 Hz, 1H), 5.60 (d, J=6.0 Hz, NH), 5.92 (s, OH), 6.91 (d, J=8.5 Hz, 1H), 7.13 (dd, J=8.5, 2.0 Hz, 1H), 7.21 (m, 2H), 7.31 (m, 4H). ¹³C NMR (100 MHz, CDCl₃): δ 28.3, 56.7, 67.5, 80.4, 116.5, 120.2, 127.2 127.8, 128.0, 128.4, 128.5, 130.0, 134.9, 151.6, 154.7, 170.6.

### (S)-benzyl 2-((tert-butoxycarbonyl)amino)-2-(4-((tert-butyldimethylsilyl)oxy)-3-chloro-phenyl)acetate 9a

A solution of 5.42 g (13.8 mmol) (S)-benzyl-2-((tert-butoxycarbonyl)amino)-2-(3-chloro-4-hydroxyphenyl)acetate 9a-II in 28 mL dry DMF was treated with 2.50 g (16.6 mmol) TBDMS-Cl and 2.35 g (34.6 mmol) imidazole at 0° C. and further stirred for 12 h. The reaction mixture was diluted with ethyl acetate and washed with 1 M HCl, satd NaHCO₃, brine, dried over Na₂SO₄ and concentrated in vacuo. Purification by flash chromatography (hexanes/ethyl acetate 9:1) resulted in the isolation of 9a (6.91 g, 13.7 mmol, 99%) as a colorless oil. R_{f}: 0.29 (hexanes/ethyl acetate 9:1). [α]²⁰_{D}=+28.4° (c=1.0, CHCl₃, er=7:3). ¹H NMR (400 MHz, CDCl₃): δ 0.22 (s, 6H), 1.03 (s, 9H), 1.43 (s, 9H), 5.16 (d, J=3.0 Hz, 2H), 5.28 (d, J=7.0 Hz, 1H), 5.52 (d, J=6.5 Hz, NH), 6.82 (d, J=8.3 Hz, 1H), 7.09 (dd, J=8.3, 2.3 Hz, 1H), 7.21 (m, 2H), 7.31 (m, 4H). ¹³C NMR (100 MHz, CDCl₃): δ −4.4, 18.3, 25.6, 28.3, 56.7, 67.4, 80.3, 120.8, 125.9, 126.4, 127.9, 128.3, 128.5, 128.9, 130.6, 135.1, 151.7, 154.7, 170.7.

### (S)-2-((tert-butoxycarbonyl)amino)-2-(4-((tert-butyldimethylsilyl)oxy)-3-chlorophenyl)acetic acid (10a)^{[1]}

A solution of 2.91 g (5.75 mmol) 9a and 146 mg Pd/C (10% on carbon) in 15 mL dry MeOH was stirred under hydrogen atmosphere (1013 hPa) for 24 h. Filtration over celite and removal of the solvent under reduced pressure resulted in the isolation of 2.36 g (5.67 mmol, 99%) protected acid 10a as a white solid. R_{f}: 0.49 (hexanes/ethyl acetate 1:1+1% AcOH). [α]²⁰_{D}=+57.8° (c=1.0, CHCl₃, er=7:3). Mp: 68-70° C. 6:3 Mixture of rotamers. ¹H NMR (400 MHz, CDCl₃): δ 0.22 (s, 6H), 1.02 (s, 9H), 1.25 (s, 6H), 1.43 (s, 3H), 5.02 (d, J=4.5 Hz, 0.7H), 5.25 (s, 0.3H), 5.47 (s, 0.3 NH), 6.85 (d, J=8.3 Hz, 1H), 7.17 (m, 1H), 7.41 (m, 1H), 8.02 (s, 0.7 NH). ¹³C NMR (100 MHz, CDCl₃): δ −4.4, 18.3, 25.6, 28.0, 28.3 (rotamere), 57.9, 81.9, 120.6, 125.5, 126.2, 129.2, 132.2, 151.3, 156.9, 173.1.

### (S)-carboxy(3-chloro-4-hydroxyphenyl)methanaminium bromide (8b)^{[2]}

To a suspension of 5.02 g (30.0 mmol) 4-Hydroxy-L-Phenylglycine in 15 mL acetic acid were added 15 mL (87.0 mmol) HBr (33% solution in acetic acid) and a solution of 1.70 mL (33.0 mmol) bromine in 10 mL acetic acid. The resulting mixture was stirred for 48 h, the precipitate was filtered and washed several times with acetic acid and diethyl ether to receive 6.56 g (19.9 mmol, 66%) 8b as a white solid. [α]²⁰_{D}=+106.6° (c=1.0, MeOH). Mp: 219-220° C. ¹H NMR (400 MHz, MeOH): 55.00 (s, 1H), 6.97 (d, J=8.5 Hz, 1H), 7.29 (dd, J=8.3, 2.3 Hz, 1H), 7.62 (d, J=2.3 Hz, 1H). ¹³C NMR (100 MHz, MeOH): δ 56.5, 111.4, 117.7, 125.9, 129.7, 134.1, 157.1, 170.7. Anal. Calc for C₈H₉Br₂NO₃ (326.97): C, 29.39; H, 2.77; N, 4.28. found C, 29.24; H, 2.36; N, 4.24.

### (S)-allyl 2-(3-bromo-4-((tert-butyldimethylsilyl)oxy)phenyl)-2-((tert-butoxycarbonyl)-amino)-acetate (9b)

### (S)-2-(3-bromo-4-hydroxyphenyl)-2-((tert-butoxycarbonyl)amino)acetic acid 9b-I^{[2]}

According to amino acid 9a-I, N-Boc protected acid 9b-I was prepared from brominated amino acid 8b (6.54 g, 19.8 mmol), 75.0 mL NaOH (1 M in H₂O), 35.0 mL dioxane and di-tert-butyl dicarbonat (5.19 g, 23.8 mmol). Drying under reduced pressure gave rise to 9b-I (6.80 g, 19.6 mmol, 99%) as a colorless oil. R_{f}: 0.36 (hexanes/ethyl acetate 1:1+1% AcOH). [α]²⁰_{D}=+112.5° (c=1.0, MeOH, er=8:2). Mp: 88-90° C. 7:2 Mixture of rotamers. ¹H NMR (400 MHz, CDCl₃): δ 1.26 (s, 7H), 1.42 (s, 2H), 5.04 (d, J=5.3 Hz, 0.7H), 5.23 (s, 0.3H), 5.66 (s, 0.3 NH), 6.96 (d, J=8.3 Hz, 1H), 7.24 (m, 1H), 7.48 (s, 0.3H), 7.54 (s, 0.7H), 8.01 (s, 0.7 NH). ¹³C NMR (100 MHz, CDCl₃): δ 28.0, 28.3 (rotamere), 56.4 (rotamere), 57.7, 80.9 (rotamere), 82.1, 110.1, 116.0, 127.9, 130.7, 131.7, 152.1, 156.9, 173.2. HRMS (Cl) m/z calc. for C₁₃H₁₇BrNO₅ (M+H)⁺: 346.0285. found 346.0263. Anal. Calc for C₁₃H₁₆BrNO₅ (346.17): C, 45.10; H, 4.66; N, 4.05. found C, 45.06; H, 4.63; N, 3.84.

### (S)-allyl 2-(3-bromo-4-hydroxyphenyl)-2-((tert-butoxycarbonyl)amino)acetate 9b-II

According to ester 9a-II, allylic ester 9b-II was prepared from N-Boc protected amino acid 9b-I (2.99 g, 8.29 mmol), 1.26 g (9.12 mmol) K₂CO₃ and 1.30 g (10.8 mmol) allyl bromide. Purification by flash chromatography (hexanes/ethyl acetate 7:3) gave rise to 9b-II (1.87 g, 4.84 mmol, 58%) as a colorless oil. R_{f}: 0.30 (hexanes/ethyl acetate 7:3). [α]²⁰_{D}=+41.4° (c=1.0, CHCl₃, er=8:2). ¹H NMR (400 MHz, CDCl₃): δ 1.43 (s, 9H), 4.62 (m, 2H), 5.22 (m, 3H), 5.57 (s, NH), 5.79 (m, 2H), 6.95 (d, J=8.3 Hz, 1H), 7.21 (dd, J=8.3, 1.8 Hz, 1H), 7.48 (d, J=2.3 Hz, 1H). ¹³C NMR (100 MHz, CDCl₃): δ 28.3, 56.6, 66.3, 80.4, 110.4, 116.3, 118.8, 127.9, 130.5, 130.7, 131.2, 152.5, 154.7, 170.5. HRMS (Cl) m/z calc. for C₁₆H₂₁BrNO₅ (M+H)⁺: 386.0598. found 386.0638.

### (S)-allyl 2-(3-bromo-4-((tert-butyldimethylsilyl)oxy)phenyl)-2-((tert-butoxycarbonyl)amino) acetate 9b

According to N-Boc protected ester 9a, ester 9b was prepared from allylic ester 9b-II (1.75 g, 4.53 mmol), 819 mg (5.44 mmol) TBDMS-Cl and 617 mg (9.06 mmol) imidazole. Purification by flash chromatography (hexanes/ethyl acetate 9:1) gave rise to 9b (2.26 g, 4.52 mmol, 100%) as a colorless oil. R_{f}: 0.33 (hexanes/ethyl acetate 9:1). [α]²⁰_{D}=+34.3° (c=1.0, CHCl₃, er=8:2). ¹H NMR (400 MHz, CDCl₃): δ 0.24 (s, 6H), 1.03 (s, 9H), 1.43 (s, 9H), 4.62 (d, J=5.5 Hz, 2H), 5.19 (m, 2H), 5.24 (d, J=7.0 Hz, 1H), 5.51 (d, J=6.0 Hz, NH), 5.83 (ddt, J=17.3, 10.0, 5.8 Hz, 1H), 6.82 (d, J=8.3 Hz, 1H), 7.17 (dd, J=8.4, 1.9 Hz, 1H), 7.52 (d, J=2.3 Hz, 1H). ¹³C NMR (100 MHz, CDCl₃): δ −4.3, 18.3, 25.7, 28.3, 56.6, 66.2, 80.3, 115.6, 118.6, 120.2, 127.2, 130.9, 131.2, 131.9, 152.8, 154.7, 170.6. HRMS (Cl) m/z calc. for C₂₂H₃₅BrNO₅Si (M+H)⁺: 500.1462. found 500.1299. Anal. Calc for C₂₂H₃₄BrNO₅Si (346.17): C, 52.79; H, 6.85; N, 2.80. found C, 53.46; H, 7.07; N, 2.94.

### (S)-2-(3-bromo-4-((tert-butyldimethylsilyl)oxy)phenyl)-2-((tert-butoxycarbonyl)amino)acetic acid (10b)^{[1]}

To a solution of 2.33 g (4.66 mmol) 9b in 30 mL dry THF were added 155 mg (134 μmol) Pd(PPh₃)₄ and 0.81 mL (9.31 mmol) morpholine. The reaction mixture was stirred overnight, diluted with ethyl acetate, washed with 1 M HCl (2×), brine, dried over Na₂SO₄ and concentrated under reduced pressure. Purification by flash chromatography (hexanes/ethyl acetate 1:1) gave rise to 10b (2.04 g, 4.43 mmol, 95%) as a white solid. R_{f}: 0.61 (hexanes/ethyl acetate 1:1+1% AcOH). [α]²⁰_{D}=+49.1° (c=1.0, CHCl₃, er=8:2). Mp: 70-72° C. 7:3 Mixture of rotamers. ¹H NMR (400 MHz, CDCl₃): δ 0.24 (s, 6H), 1.04 (s, 9H), 1.26 (s, 7H), 1.44 (s, 2H), 5.02 (d, J=5.0 Hz, 0.8H), 5.25 (s, 0.2H), 5.48 (s, 0.2 NH), 6.84 (d, J=8.5 Hz, 1H), 7.24 (m, 1H), 7.59 (m, 1H), 8.08 (s, 0.8 NH). ¹³C NMR (100 MHz, CDCl₃): δ −4.2, 18.3, 25.7, 28.0, 57.7, 81.9, 115.2, 120.1, 126.9, 132.3, 132.3, 152.4, 156.9, 173.1. HRMS (Cl) m/z calc. for C₁₉H₃₁BrNO₅Si (M+H)⁺: 458.1004. found 458.1044. Anal. Calc for C₁₉H₃₀BrNO₅Si (460.43): C, 49.56; H, 6.57; N, 3.04. found C, 50.54; H, 6.56; N, 3.02.

### (S)-tert-butyl-(1-(4-((tert-butyldimethylsilyl)oxy)-3-chlorophenyl)-3-diazo-2-oxopropyl)-carbamate (11a)

Preparation of diazomethane: To a mixture of 20.0 mL (5.87 mmol) KOH (40% in H₂O) and 80 mL diethyl ether was added 1.21 g (11.7 mmol) 1-methyl-1-nitrosourea in several portions at −10° C. (Internal temperature may not increase over −5° C.). The reaction mixture was cooled to −78° C. and the organic layer was decanted.

Reaction: To a solution of 2.52 g (5.87 mmol) N-Boc protected amino acid 10a in 100 mL dry THF were added 0.90 mL (6.46 mmol) triethylamine and 0.62 mL (6.46 mmol) ethyl chloroformate at −20° C. After 20 min at this temperature, the suspension was cooled to −78° C. and the fresh prepared diazomethane was added. The reaction mixture was allowed to warm up to room temperature overnight. After addition of H₂O, the layers were separated and extracted with ethyl acetate. The combined organic layers were further washed with satd NaHCO₃, H₂O, dried over Na₂SO₄ and concentrated under reduced pressure. Purification by flash chromatography (hexanes/ethyl acetate 8:2) resulted in the isolation of 11a (2.06 g, 4.68 mmol, 80%) as a yellow solid. R_{f}: 0.23 (hexanes/ethyl acetate 8:2). [α]²⁰_{D}=+116.2° (c=1.0, CHCl₃, er=3:7). Mp: 51-53° C. ¹H NMR (400 MHz, CDCl₃): δ 0.22 (s, 6H), 1.01 (s, 9H), 1.41 (s, 9H), 5.08 (s, 1H), 5.24 (s, 1H), 5.82 (d, J=5.3 Hz, NH), 6.84 (d, J=8.3 Hz, 1H), 7.07 (d, J=7.0 Hz, 1H), 7.29 (d, J=2.3 Hz, 1H). ¹³C NMR (100 MHz, CDCl₃): δ −4.4, 18.3, 25.6, 28.3, 54.7, 60.7, 80.2, 120.9, 126.2, 126.8, 129.4, 131.1, 151.8, 154.9, 190.4.

### (R,E)-allyl-7-((3-((tert-butoxycarbonyl)amino)-3-(4-((tert-butyldimethylsilyl)oxy)-3-chloro-phenyl)propanoyl)oxy)-4-methylhept-4-enoate (12a)

To a solution of 1.99 g (4.52 mmol) diazoketon 11a and 1.79 g (9.04 mmol) unsaturated hydroxy ester 7a in 4.5 mL dry THF was added a solution of 114 mg (0.49 mmol) silver benzoate in 1.83 mL (13.1 mmol) triethylamine in the dark at 25° C. The reaction mixture was allowed to warm up to room temperature overnight. The precipitate was filtered, diluted with ethyl acetate and the organic layer was washed with 1 M Na₂S₂O₃, satd NaHCO₃, H₂O, satd NH₄Cl, brine and dried over Na₂SO₄. Removal of the solvent under reduced pressure and purification by flash chromatography (hexanes/ethyl acetate 8:2) resulted in the isolation of 12a (2.25 g, 3.69 mmol, 82%) as a colorless oil. R_{f}: 0.23 (hexanes/ethyl acetate 8:2). [α]²⁰_{D}=+10.6° (c=1.0, CHCl₃, er=7:3). ¹H NMR (400 MHz, CDCl₃): δ 0.20 (s, 6H), 1.01 (s, 9H), 1.42 (s, 9H), 1.61 (s, 3H), 2.23 (td, J=7.2, 7.2 Hz, 2H), 2.32 (m, 2H), 2.43 (m, 2H), 2.74 (dd, J=15.3, 5.7 Hz, 1H), 2.82 (dd, J=14.8, 5.8 Hz, 1H), 3.97 (t, J=5.8 Hz, 2H), 4.56 (ddd, J=5.8, 1.5, 1.5 Hz, 2H), 4.99 (s, 1H), 5.08 (t, J=6.5 Hz, 1H), 5.22 (ddt, J=10.5, 1.25, 1.25 Hz, 1H), 5.30 (ddt, J=17.3, 1.5, 1.5 Hz, 1H), 5.48 (s, NH), 5.90 (ddt, J=17.1, 11.5, 5.8 Hz, 1H), 6.82 (d, J=8.5 Hz, 1H), 7.04 (dd, J=8.4, 2.1 Hz, 1H), 7.27 (d, J=2.3 Hz, 1H). ¹³C NMR (100 MHz, CDCl₃): δ −4.4, 16.0, 18.3, 25.6, 27.3, 28.3, 32.9, 34.5, 40.7, 50.2, 64.2, 65.0, 79.8, 118.2, 119.7, 120.6, 125.4, 125.6, 128.0, 132.2, 132.2, 136.7, 150.8, 154.9, 170.7, 172.9. HRMS (Cl) m/z calc. for C₃₁H₄₉ClNO₇Si (M+H)⁺: 610.2961. found 610.2874. Anal. Calc for C₃₁H₄₈ClNO₇Si (610.25): C, 61.01; H, 7.93; N, 2.30. found C, 60.05; H, 8.06; N, 2.77. HPLC (Reprosil, Hex:iPrOH, 15 min at 8:2, than in 15 min. to 7:3, 1 mL/min): t_{R(major)}=9.48 min, t_{R(minor)}=23.28 min.

### (S)-tert-butyl-(1-(3-bromo-4-((tert-butyldimethylsilyl)oxy)phenyl)-3-diazo-2-oxopropyl)-carbamate (11b)

Preparation of diazomethane: To a mixture of 2.7 mL (0.73 mmol) KOH (40% in H₂O) and 10 mL diethyl ether was added 150 mg (1.46 mmol) 1-methyl-1-nitrosourea in several portions at −10° C. (Internal temperature may not increase over −5° C.). The reaction mixture was cooled to −78° C. and the organic layer was decanted.

Reaction: To a solution of 335 mg (0.73 mmol) N-Boc protected amino acid 10b in 12 mL dry THF were added 0.11 mL (0.80 mmol) triethylamine and 77.0 μL (0.80 mmol) ethyl chloroformate at −20° C. After 20 min at this temperature, the suspension was cooled to −78° C. and the fresh prepared diazomethane was added. The reaction mixture was allowed to warm up to −20° C. and was stirred overnight at this temperature. The excess of diazomethane was destroyed with addition of 1 mL AcOH. After addition of H₂O, the layers were separated and extracted with ethyl acetate. The combined organic layers were further washed with satd NaHCO₃, H₂O, dried with Na₂SO₄ and concentrated under reduced pressure. Purification by flash chromatography (hexanes/ethyl acetate 8:2) resulted in the isolation of 11b (258 mg, 0.53 mmol, 73%) as a yellow solid. R_{f}: 0.24 (hexanes/ethyl acetate 8:2). [α]²⁰_{D}=+160.5° (c=1.0, CHCl₃, er=2:8). Mp: 53-55° C. ¹H NMR (400 MHz, CDCl₃): δ 0.24 (s, 6H), 1.02 (s, 9H), 1.41 (s, 9H), 5.07 (s, 1H), 5.23 (s, 1H), 5.81 (s, NH), 6.83 (d, J=8.3 Hz, 1H), 7.12 (d, J=7.3 Hz, 1H), 7.46 (d, J=2.3 Hz, 1H). ¹³C NMR (100 MHz, CDCl₃): δ −4.2, 18.3, 25.6, 28.3, 54.7, 60.6, 80.2, 115.9, 120.3, 127.6, 131.4, 132.4, 152.9, 154.9, 190.4. HRMS (Cl) m/z calc. for C₂₀H₃₁BrN₃O₄Si (M+H)⁺: 484.1262. found 484.1212.

### (R,E)-allyl-7-((3-(3-bromo-4-((tert-butyldimethylsilyl)oxy)phenyl)-3-((tert-butoxycarbonyl)-amino)propanoyl)oxy)-4-methylhept-4-enoate (12b)

According to ester 12a, β-tyrosine-derivative 12b was prepared from diazoketon 11b (310 mg, 640 μmol), unsaturated hydroxy ester 7 (270 mg, 1.36 mmol), triethylamine (259 μL, 1.86 mmol) and silver benzoate (16.0 mg, 70.0 μmol). Purification by flash chromatography (hexanes/ethyl acetate 8:2) gave rise to 12b (266 mg, 406 μmol, 63%) as a yellow oil. R_{f}: 0.31 (hexanes/ethyl acetate 8:2). [α]²⁰_{D}=+14.4° (c=1.0, CHCl₃, er=8:2). ¹H NMR (400 MHz, CDCl₃): δ 0.23 (s, 6H), 1.03 (s, 9H), 1.42 (s, 9H), 1.61 (s, 3H), 2.24 (td, J=7.0, 7.0 Hz, 2H), 2.33 (m, 2H), 2.44 (m, 2H), 2.74 (dd, J=15.5, 6.0 Hz, 1H), 2.81 (dd, J=15.3, 6.0 Hz, 1H), 3.97 (td, J=7.0, 1.5 Hz, 2H), 4.56 (ddd, J=5.6, 1.25, 1.25 Hz, 2H), 4.99 (s, 1H), 5.08 (t, J=7.8 Hz, 1H), 5.22 (ddt, J=10.3, 1.25, 1.25 Hz, 1H), 5.30 (ddt, J=17.3, 1.5, 1.5 Hz, 1H), 5.45 (s, NH), 5.91 (ddt, J=17.1, 10.3, 5.8 Hz, 1H), 6.80 (d, J=8.3 Hz, 1H), 7.09 (dd, J=8.5, 2.3 Hz, 1H), 7.44 (d, J=2.3 Hz, 1H). ¹³C NMR (100 MHz, CDCl₃): δ −4.3, 16.0, 18.3, 25.7, 27.3, 28.3, 32.9, 34.5, 40.8, 55.7, 64.2, 65.0, 79.8, 115.4, 118.2, 119.8, 120.0, 126.2, 131.1, 132.2, 132.2, 136.7, 151.8, 154.9, 170.7, 172.8. HRMS (Cl) m/z calc. for C₃₁H₄₉BrNO₇Si (M+H)⁺: 654.2456. found 654.2462. HPLC (Reprosil, Hex:iPrOH, 15 min at 8:2, than in 15 min to 7:3, 1 mL/min): t_{R(major)}=9.80 min, t_{R(minor)}=25.76 min.

### Syntheses of Chondramides

Different chondramides can easily be obtained by combining various peptide fragments with various hydroxy carboxylic acids. Two different ring closing approaches are described in the literature.

### Route 1:^{[3]}

Synthesis of the required tripeptide and coupling the hydroxy carboxylic acid to the N-terminus of the tripeptide. Subsequent ring closure via cycloesterification.

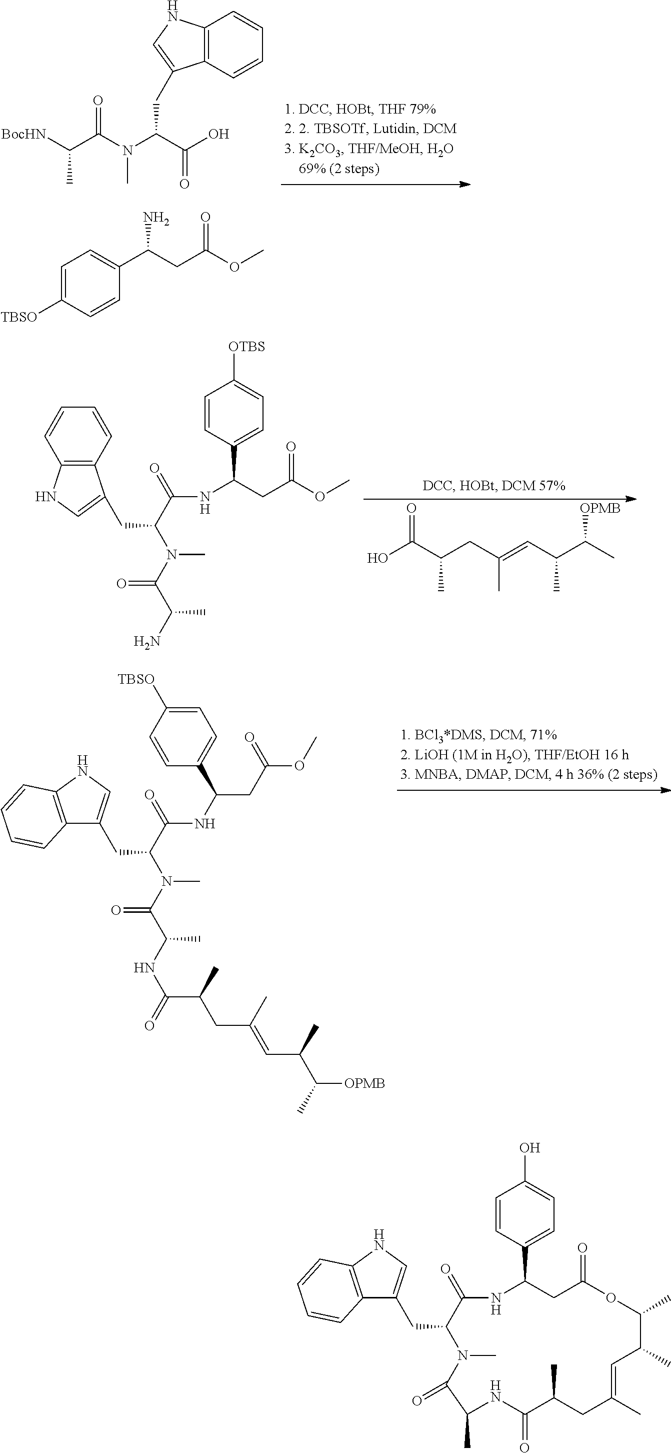

### Route 2:^{[7]}

Synthesis of the required tripeptide and coupling the hydroxy carboxylic acid to the C-terminus of the tripeptide. Subsequent ring closure via cycloamidation.

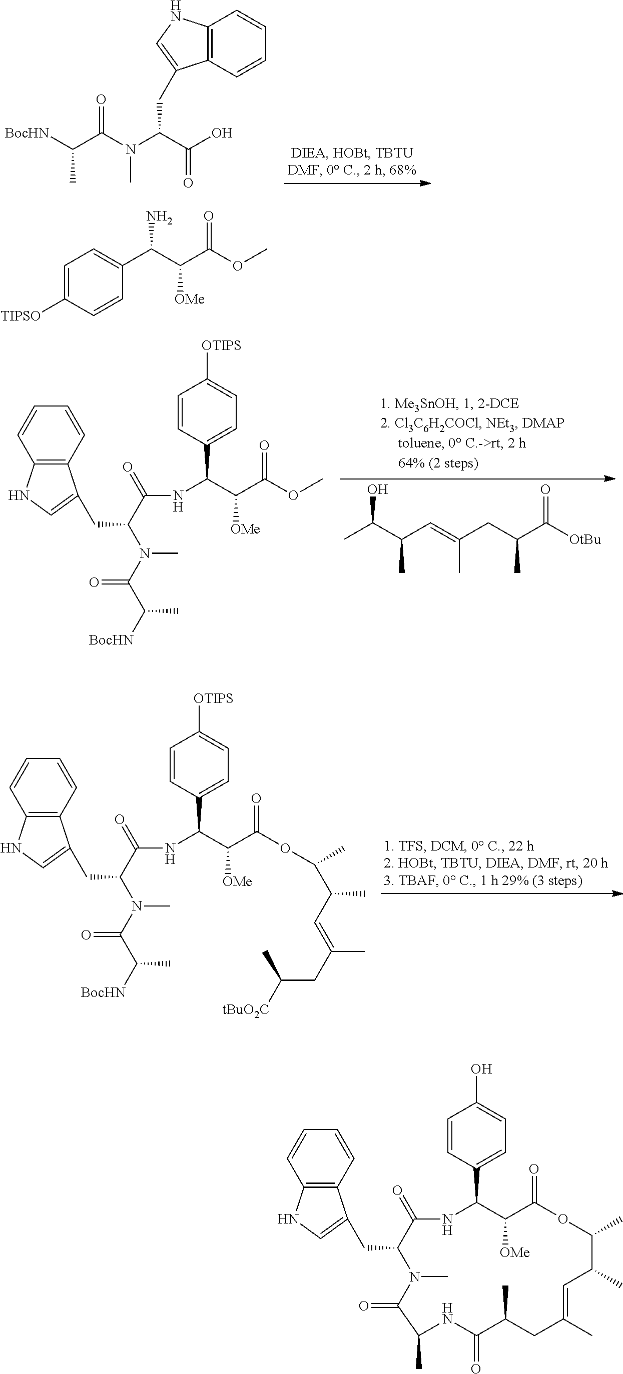

Our own synthesis starts with coupling of the (halogenated) dipeptide with the halogenated β-tyrosine-hydroxy carboxylic acid fragment. As illustrated for the simplified hydroxy acid, ring closure can be performed via cycloamidation.

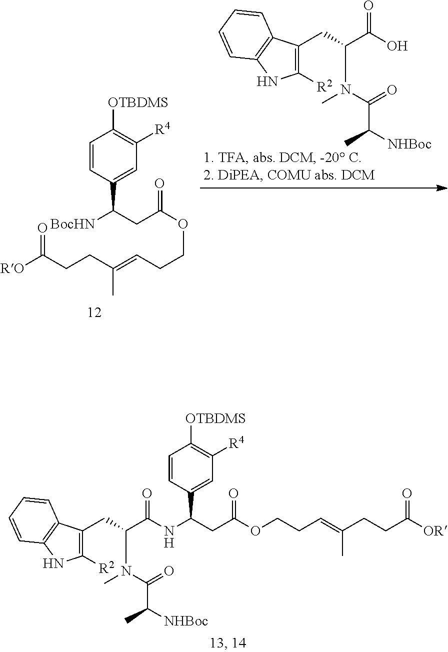

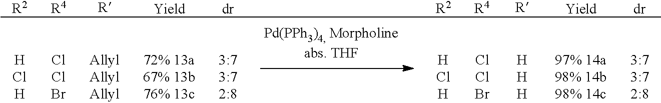

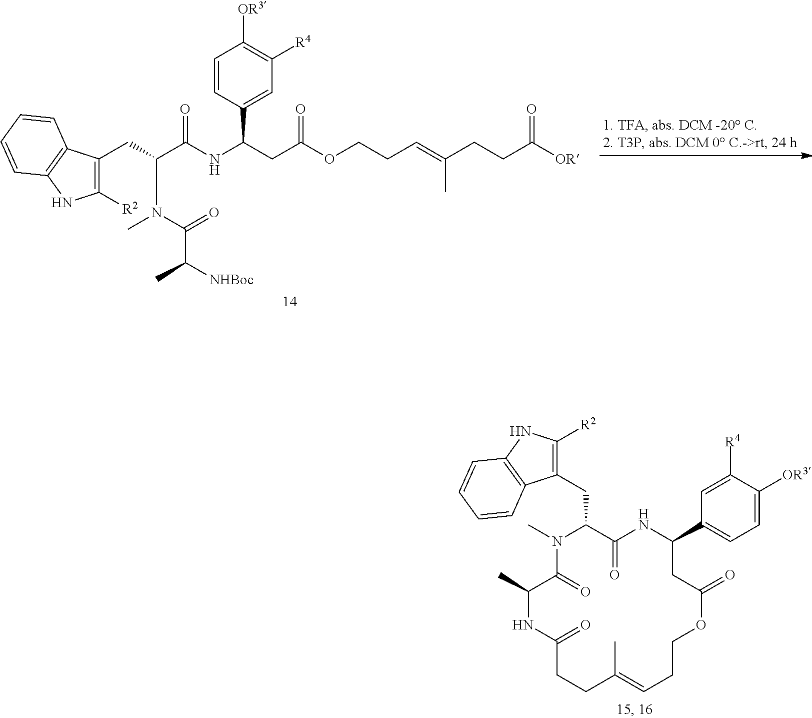

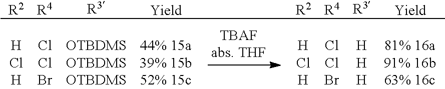

### (6S,9R,12R)-(E)-7-(allyloxy)-4-methyl.-7-oxohept-3-en-1-yl 9-((1H-indol-3-yl)methyl)-12-(4-((tert-butyldimethylsilyl)oxy)-3-chlorophenyl)-2,2,6,8-tetramethyl-4,7,10-trioxo-3-oxa-5,8,11-triazatetradecan-14-oate (13a)

A solution of 430 mg (705 μmol) β-tyrosine-derivative 12a in 5 mL dry THF was treated with 2.20 mL (28.6 mmol) trifluoroacetic acid at −20° C. and was stirred for 12 h at this temperature. The reaction mixture was poured into a sat. NaHCO₃ solution and extracted with DCM (3×), the combined organic layer was further washed with brine, dried over Na₂SO₄ and concentrated in vacuo. To a solution of the resulted crude amine in 2 mL dry DCM were added 329 mg (846 μmol) (R)-2-((S)-2-((tert-butoxycarbonyl)amino)-N-methyl propanamido)-3-(1H-indol-3-yl)propanoic acid, 0.22 mL (1.27 mmol) DiPEA and 362 mg (846 μmol) COMU® at 0° C. The mixture was allowed to warm up to room temperature overnight, concentrated to dryness and the residue was diluted in ethyl acetate. The organic layer was further washed with 1. M KHSO₄, H₂O, satd NaHCO₃, brine, dried over Na₂SO₄ and concentrated under reduced pressure. Purification by flash chromatography (hexanes/ethyl acetate 6:4) resulted in the isolation of 13a (450 mg, 0.51 mmol, 72%, dr=3:7) as a yellow foam. R_{f}: 0.33 (hexanes/ethyl acetate 1:1). [α]²⁰_{D}=+16.3° (c=1.0, CHCl₃, dr=3:7). 3:7 Mixture of diastereomers. ¹H NMR (400 MHz, CDCl₃), major diastereomer: δ 0.21 (s, 6H), 0.94 (d, J=6.5 Hz, 3H), 1.01 (s, 9H), 1.40 (s, 9H), 1.61 (s, 3H), 2.23 (m, 2H), 2.31 (m, 2H), 2.43 (m, 2H), 2.72 (dd, J=13.0, 7.3 Hz, 1H), 2.85 (dd, J=15.6, 8.0 Hz, 1H), 2.92 (s, 3H), 3.21 (dd, J=15.8, 10.3 Hz, 1H), 3.46 (dd, J=15.6, 5.8 Hz, 1H), 3.96 (t, J=7.0 Hz, 2H), 4.38 (m, 1H), 4.56 (d, J=5.5 Hz, 2H), 5.08 (m, 1H), 5.22 (d, J=11.3 Hz, 1H), 5.31 (m, 2H, NH), 5.60 (m, 1H), 5.89 (ddt, J=17.1, 10.5, 5.8 Hz, 1H), 6.77 (d, J=8.3 Hz, 1H), 6.99 (m, 2H), 7.12 (m, 3H, NH), 7.32 (d, J=7.8 Hz, 1H), 7.59 (d, J=7.8 Hz, 1H), 8.20 (s, NH). Minor diastereomer (selected signals): δ 1.01 (s, 9H), 1.44 (s, 9H), 1.59 (s, 3H), 2.77 (s, 3H), 3.14 (m, 1H), 3.40 (m, 1H), 3.91 (m, 2H), 6.81 (d, J=8.5 Hz, 1H). ¹³C NMR (100 MHz, CDCl₃): δ −4.3, 16.1, 16.9, 18.3, 23.2, 25.6, 27.4, 28.3, 30.9, 32.9, 34.5, 40.3, 46.6, 49.1, 56.7, 64.2, 65.1, 79.9, 111.1, 111.1, 118.2, 118.6, 119.4, 119.8, 120.6, 122.1, 122.7, 125.6, 125.7, 127.4, 128.2, 132.2, 134.7, 136.1, 136.6, 150.8, 155.7, 169.4, 170.5, 172.9, 174.3. HRMS (Cl) m/z calc. for C₄₆H₆₆ClN₄O₉Si (M+H)⁺: 881.4284. found 881.4334. HPLC (Reprosil, Hex:iPrOH, 10 min at 3:7, than in 5 min to 1:9, than 15 min at 1:9, 1 mL/min, 25° C.): t_{R(major)}=11.93 min, t_{R(minor)}=26.95 min.

### (6S,9R,12R)-(E)-7-(allyloxy)-4-methyl-7-oxohept-3-en-1-yl 12-(4-((tert-butyldimethylsilyl)oxy)-3-chlorophenyl)-9-((2-chloro-1H-indol-3-yl)methyl)-2,2,6,8-tetramethyl-4,7,10-trioxo-3-oxa-5,8,11-triazatetradecan-14-oate (13b)

According to tripeptide 13a, double chlorinated tripeptide 13b was prepared from β-tyrosine-derivative 12b (304 mg, 498 μmol), chlorinated dipeptide 5 (205 mg, 484 μmol), trifluoroacetic acid (1.50 mL, 19.4 mmol), DiPEA (0.15 mL, 0.87 mmol) and COMU® (249 mg, 0.58 mmol). Purification by flash chromatography (hexanes/ethyl acetate 7:3) gave rise to 13b (298 mg, 325 μmol, 67%, dr=3:7) as a brown foam. R_{f}: 0.22 (hexanes/ethyl acetate 7:3). [α]²⁰_{D}=+30.1° (c=1.0, CHCl₃, dr=3:7). 3:7 Mixture of diastereomers. ¹H NMR (400 MHz, CDCl₃), major diastereomer: δ 0.21 (s, 6H), 0.67 (d, J=6.8 Hz, 3H), 1:02 (s, 9H), 1.37 (s, 9H), 1.61 (s, 3H), 2.24 (m, 2H), 2.31 (m, 2H), 2.43 (t, J=7.1 Hz, 2H), 2.74 (dd, J=15.8, 5.8 Hz, 1H), 2.86 (dd, J=15.8, 8.0 Hz, 1H), 2.96 (s, 3H), 3.21 (dd, J=15.3, 10.8 Hz, 1H), 3.40 (dd, J=15.1, 5.3 Hz, 1H), 3.96 (t, J=7.0 Hz, 2H), 4.28 (q, J=6.8 Hz, 1H), 4.56 (d, J=4.5 Hz, 2H), 5.11 (m, 1H, NH), 5.22 (d, J=10.3 Hz, 1H), 5.34 (m, 2H), 5.65 (dd, J=10.8, 5.6 Hz, 1H), 5.91 (ddt, J=17.1, 10.5, 5.8 Hz, 1H), 6.78 (d, J=8.5 Hz, 1H), 7.00 (m, 2H), 7.09 (m, 2H), 7.21 (m, 1H, NH), 7.49 (d, J=7.8 Hz, 1H), 8.32 (s, NH). Minor diastereomer (selected signals): δ 0.20 (s, 6H), 1.01 (s, 9H), 1.40 (s, 9H), 2.80 (s, 3H), 3.15 (m, 1H), 3.35 (m, 1H), 3.93 (t, J=7.0 Hz, 2H), 6.81 (d, J=8.5 Hz, 1H), 8.40 (s, NH). ¹³C NMR (100 MHz, CDCl₃): δ −4.4, 16.0, 16.4, 18.3, 22.2, 25.6, 27.3, 28.3, 31.5, 32.9, 34.5, 40.3, 46.4, 49.1, 56.2, 64.2, 65.0, 79.8, 107.3, 110.4, 118.2, 118.3, 119.7, 120.2, 120.6, 121.6, 122.3, 125.6, 125.7, 127.3, 128.2, 132.2, 134.4, 134.6, 136.6, 150.9, 155.7, 169.2, 170.4, 172.9, 174.3. HRMS (Cl) m/z calc. for C₄₆H₆₅Cl₂N₄O₉Si (M+H)⁺: 915.3892. found 915.3930. HPLC (Reprosil, Hex:iPrOH, 30 min at 40:60, 1 mL/min, 25° C.): t_{R(major)}=10.35 min, t_{R(minor)}=26.15 min.

### (6S,9R,12R)-(E)-7-(allyloxy)-4-methyl-7-oxohept-3-en-1-yl-9-((1H-indol-3-yl)methyl)-12-(3-bromo-4-((tert-butyldimethylsilyl)oxy)phenyl)-2,2,6,8-tetramethyl-4,7,10-trioxo-3-oxa-5,8,11-triazatetradecan-14-oate (13c)

According to tripeptide 13a, brominated tripeptide 13c was prepared from β-tyrosine-derivative 12c (253 mg, 386 μmol), (R)-2-((S)-2-((tert-butoxycarbonyl)amino)-N-methylpropanamido)-3-(1H-indol-3-yl)propanoic acid (181 mg, 464 μmol), trifluoroacetic acid (1.20 mL, 15.6 mmol), DiPEA (0.12 mL, 0.69 mmol) and COMU® (199 mg, 0.46 mmol). Purification by flash chromatography (hexanes/ethyl acetate 7:3) gave rise to 13c (273 mg, 295 μmol, 76%, dr=2:8) as a brown foam. R_{f}: 0.30 (hexanes/ethyl acetate 1:1). [α]²⁰_{D}=+11.4° (c=1.0, CHCl₃, dr=2:8). 2:8 Mixture of diastereomers. ¹H NMR (400 MHz, CDCl₃), major diastereomer: δ 0.22 (s, 6H), 0.94 (d, J=6.8 Hz, 3H), 1.03 (s, 9H), 1.40 (s, 9H), 1.61 (s, 3H), 2.25 (m, 2H), 2.31 (m, 2H), 2.43 (t, J=8.0 Hz, 2H), 2.73 (dd, J=15.6, 5.8 Hz, 1H), 2.85 (dd, J=16.5, 8.8 Hz, 1H), 2.92 (s, 3H), 3.26 (dd, J=15.8, 10.3 Hz, 1H), 3.45 (dd, J=15.8, 5.8 Hz, 1H), 3.97 (t, J=7.7 Hz, 2H), 4.38 (m, 1H), 4.56 (d, J=7.0 Hz, 2H), 5.09 (t, J=8.3 Hz, 1H), 5.22 (d, J=10.5 Hz, 1H), 5.31 (m, 2H, NH), 5.60 (dd, J=10.0, 5.8 Hz, 1H), 5.89 (ddt, J=17.1, 10.3, 5.8 Hz, 1H), 6.75 (d, J=8.5 Hz, 1H), 6.96 (s, 1H), 7.07 (m, 2H, NH), 7.16 (dd, J=8.2, 8.2 Hz, 1H), 7.32 (d, J=8.0 Hz, 1H), 7.38 (s, 1H), 7.59 (d, J=8.0 Hz, 1H), 8.16 (s, NH). Minor diastereomer (selected signals): δ 1.02 (s, 9H), 1.44 (s, 9H), 1.59 (s, 3H), 2.78 (s, 3H), 3.93 (t, J=7.3 Hz, 2H), 6.79 (d, J=8.3 Hz, 1H), 8.26 (s, NH). ¹³C NMR (100 MHz, CDCl₃): δ −4.3, 16.0, 17.0, 18.3, 23.2, 25.7, 27.3, 28.3, 30.8, 32.9, 34.5, 40.3, 46.6, 49.0, 56.7, 64.1, 65.0, 79.8, 111.1, 111.1, 115.3, 118.2, 118.5, 119.4, 119.8, 120.0, 122.0, 122.0, 126.4, 127.4, 131.2, 132.2, 134.9, 136.1, 136.5, 151.9, 155.6, 169.4, 170.4, 172.9, 174.3. HRMS (Cl) m/z calc. for C₄₆H₆₆BrN₄O₉Si (M+H)⁺: 925.3777. found 925.3818. HPLC (Reprosil, Hex:iPrOH, 10 min at 3:7, than in 5 min to 1:9, than 15 min at 1:9, 1 mL/min, 25° C.): t_{R(major)}=12.88 min, t_{R(minor)}=30.25 min.

### (6S,9R,12R,E)-9-((1H-indol-3-yl)methyl)-12-(4-((tert-butyldimethylsilyl)oxy)-3-chlorophenyl)-2,2,6,8,19-pentamethyl-4,7,10,14-tetraoxo-3,15-dioxa-5,8,11-triazadocos-18-en-22-oic acid (14a)

A solution of 436 mg (495 μmol) tripeptide 13a in 10 mL dry THF was treated with 57.0 mg (49.0 μmol) Pd(PPh₃)₄ and 86.0 μL (0.99 mmol) morpholine at room temperature. After 60 min, ethyl acetate was added, the reaction mixture was washed with 1 M HCl, brine, dried over Na₂SO₄ and concentrated in vacuo. Purification by flash chromatography (hexanes/ethyl acetate 6:4->1:1+1% AcOH) gave rise to 14a (402 mg, 478 μmol, 97%, dr=3:7) as a yellow foam. R_{f}: 0.26 (hexanes/ethyl acetate 1:1+1% AcOH). [α]²⁰_{D}=+10.2° (c=1.0, CHCl₃, dr=3:7). 3:7 Mixture of diastereomers. ¹H NMR (400 MHz, CDCl₃), major diastereomer: δ 0.21 (s, 6H), 0.97 (d, J=6.8 Hz, 3H), 1.02 (s, 9H), 1.41 (s, 9H), 1.64 (s, 3H), 2.28 (m, 4H), 2.45 (m, 2H), 2.68 (m, 1H), 2.86 (dd, J=16.0, 9.3 Hz, 1H), 2.99 (s, 3H), 3.20 (dd, J=15.3, 9.5 Hz, 1H), 3.37 (dd, J=14.8, 6.3 Hz, 1H), 3.98 (m, 2H), 4.44 (m, 1H), 5.17 (t, J=7.0 Hz, 1H), 5.31 (m, 1H), 5.49 (m, 1H), 6.77 (d, J=8.5 Hz, 1H), 6.92 (s, 1H), 6.99 (s, NH), 7.01 (dd, J=8.5, 2.3 Hz, 1H), 7.09 (dd, J=7.9, 7.9 Hz, 1H), 7.16 (dd, J=7.5, 7.5 Hz, 1H), 7.21 (d, J=2.0 Hz, 1H), 7.31 (d, J=8.3 Hz, 1H), 7.57 (d, J=7.8 Hz, 1H), 8.09 (s, NH). Minor diastereomer (selected signals): δ 0.20 (s, 6H), 0.91 (d, J=7.0 Hz, 1H), 1.01 (s, 9H), 1.43 (s, 9H), 1.62 (s, 3H), 2.80 (s, 3H), 3.15 (m, 1H), 3.42 (m, 1H), 5.14 (t, J=6.5 Hz, 1H), 6.82 (d, J=8.5 Hz, 1H), 8.16 (s, NH). ¹³C NMR (100 MHz, CDCl₃): δ −4.4, 15.9, 17.5, 18.3, 23.4, 25.6, 27.2, 28.3, 30.9, 32.8, 34.7, 40.1, 46.8, 49.2, 56.7, 64.2, 79.8, 110.7, 111.1, 118.5, 119.4, 120.6, 120.6, 122.0, 122.3, 125.5, 125.7, 127.2, 128.2, 134.9, 136.1, 136.1, 150.8, 155.5, 169.4, 170.4, 172.9, 174.3. HRMS (Cl) m/z calc. for C₄₃H₆₂ClN₄O₉Si (M+H)⁺: 839.3824. found 839.3816. LCMS (Luna 3μ C18, 50×4.6 mm, H₂O:ACN 30:70, 20 min, rt, 0.6 mL/min): t_{R(major)}=13.652 min @ m/z [+Na]⁺864, t_{R(minor)}=14.400 min @ m/z [+Na]⁺ 864.

### (6S,9R,12R,E)-12-(4-((tert-butyldimethylsilyl)oxy)-3-chlorophenyl)-9-((2-chloro-1H-indol-3-yl)methyl)-2,2,6,8,19-pentamethyl-4,7,10,14-tetraoxo-3,15-dioxa-5,8,11-triazadocos-18-en-22-oic acid (14b)

According to acid 14a, double chlorinated acid 14b was prepared from tripeptide 13b (263 mg, 287 μmol), Pd(PPh₃)₄ (33.0 mg, 29.0 μmol) and morpholine (50.0 μL, 574 μmol). Purification by flash chromatography (hexanes/ethyl acetate 8:2->1:1+1% AcOH) gave rise to 14b (247 mg, 282 μmol, 98%, dr=3:7) as a white foam. R_{f}: 0.22 (hexanes/ethyl acetate 7:3). R_{f}: 0.14 (hexanes/ethyl acetate 7:3+1% AcOH). [α]²⁰_{D}=+19.4° (c=1.0, CHCl₃, dr=3:7). 3:7 Mixture of diastereomers. ¹H NMR (400 MHz, CDCl₃), major diastereomer: δ 0.22 (s, 6H), 0.70 (d, J=6.0 Hz, 3H), 1.02 (s, 9H), 1.38 (s, 9H), 1.64 (s, 3H), 2.31 (m, 4H), 2.46 (m, 2H), 2.66 (m, 1H), 2.86 (dd, J=16.3, 9.3 Hz, 1H), 3.01 (s, 3H), 3.22 (m, 1H), 3.33 (dd, J=15.3, 5.5 Hz, 1H), 3.98 (m, 2H), 4.37 (t, J=5.5 Hz, 1H), 5.18 (t, J=7.3 Hz, 1H), 5.30 (m, 1H), 5.64 (m, 1H), 6.77 (d, J=8.3 Hz, 1H), 7.08 (m, 3H), 7.21 (m, 1H, NH), 7.35 (m, 1H), 7.48 (d, J=7.8 Hz, 1H), 8.23 (s, NH). Minor diastereomer (selected signals): δ 0.21 (s, 6H), 0.65 (d, J=7.0 Hz, 3H), 1.01 (s, 9H), 1.40 (s, 9H), 2.82 (s, 3H), 4.29 (t, J=7.0 Hz, 1H), 6.82 (d, J=8.5 Hz, 1H), 8.29 (s, NH). ¹³C NMR (100 MHz, CDCl₃): δ −4.3, 15.9, 17.1, 18.3, 22.5, 25.6, 27.2, 28.3, 31.3, 32.9, 34.7, 40.2, 46.6, 49.3, 55.7, 64.2, 79.8, 110.4, 110.4, 118.3, 120.2, 120.6, 121.0, 121.7, 122.4, 125.5, 125.8, 127.3, 128.2, 134.3, 134.8, 136.1, 150.8, 155.5, 169.3, 170.3, 174.2, 175.5. HRMS (Cl) m/z calc. for C₄₃H₆₁Cl₂N₄O₉Si (M+H)⁺: 875.3579. found 875.3622. LCMS (Luna 3μ C18, 50×4.6 mm, H₂O:ACN 30:70, 20 min, rt, 0.6 mL/min): t_{R(major)}=16.969 min @ m/z [+Na]⁺897, t_{R(minor)}=18.556 min @ m/z [+Na]⁺897.

### (6S,9R,12R,E)-9-((1H-indol-3-yl)methyl)-12-(3-bromo-4-((tert-butyldimethylsilyl)oxy)phenyl)-2,2,6,8,19-pentamethyl-4,7,10,14-tetraoxo-3,15-dioxa-5,8,11-triazadocos-18-en-22-oic acid (14c)

According to acid 14a, brominated acid 14c was prepared from tripeptide 13c (246 mg, 266 μmol), Pd(PPh₃)₄ (31.0 mg, 27.0 μmol) and morpholine (46.0 μL, 531 μmol). Purification by flash chromatography (hexanes/ethyl acetate 6:4->1:1+1% AcOH) gave rise to 14c (230 mg, 260 μmol, 98%, dr=2:8) as a yellow foam. R_{f}: 0.29 (hexanes/ethyl acetate 1:1+1% AcOH). [α]²⁰_{D}=+6.2° (c=1.0, CHCl₃, dr=2:8). 2:8 Mixture of diastereomers. ¹H NMR (400 MHz, CDCl₃), major diastereomer: δ 0.23 (s, 6H), 0.96 (d, J=7.6 Hz, 3H), 1.03 (s, 9H), 1.41 (s, 9H), 1.64 (s, 3H), 2.36 (m, 6H), 2.68 (m, 1H), 2.85 (dd, J=16.2, 9.4 Hz, 1H), 2.98 (s, 3H), 3.20 (dd, J=15.6, 9.5 Hz, 1H), 3.37 (dd, J=15.2, 6.4 Hz, 1H), 3.98 (m, 2H), 4.47 (m, 1H), 5.19 (t, J=7.0 Hz, 1H), 5.29 (m, 1H), 5.50 (m, 1H), 6.76 (d, J=8.3 Hz, 1H), 6.92 (s, 1H), 7.12 (m, 3H), 7.34 (m, 2H, NH), 7.57 (d, J=7.8 Hz, 1H), 8.13 (s, NH). Minor diastereomer (selected signals): δ 0.22 (s, 6H), 1.02 (s, 9H), 1.43 (s, 9H), 1.62 (s, 3H), 2.80 (s, 3H), 6.80 (d, J=8.3 Hz, 1H). ¹³C NMR (100 MHz, CDCl₃): δ −4.2, 15.9, 17.5, 18.3, 23.4, 25.7, 27.2, 28.3, 30.9, 32.8, 34.7, 40.1, 46.7, 49.1, 56.7, 64.2, 79.8, 110.7, 111.1, 115.3, 118.5, 119.4, 119.9, 121.0, 122.0, 122.2, 126.5, 127.2, 131.2, 135.2, 136.0, 136.2, 151.8, 155.5, 169.4, 170.4, 174.3, 175.6. HRMS (Cl) m/z calc. for C₄₃H₆₂BrN₄O₉Si (M+H)⁺: 885.3464. found 885.3493. LCMS (Luna 3μ C18, 50×4.6 mm, H₂O:ACN 30:70, 20 min, rt, 0.6 mL/min): t_{R(major)}=14.577 min @ m/z [+Na]⁺ 907, t_{R(minor)}=15.149 min @ m/z [+Na]⁺ 907.

### (4R,7R,10S,E)-7-((1H-indol-3-yl)methyl)-4-(4-((tert-butyldimethylsilyl)oxy)-3-chlorophenyl)-8,10,15-trimethyl-1-oxa-5,8,11-triazacyclooctadec-15-ene-2,6,9,12-tetraone (15a)

A solution of 250 mg (297 μmol) tripeptide 14a in 2.6 mL dry DCM was treated with 1.20 mL (15.1 mmol)-trifluoroacetic acid at −20° C. and was stirred for 12 h at this temperature. The solvent was removed under reduced pressure and to a solution of the resulted crude amine in 150 mL dry DCM were added 0.21 mL (1.19 mmol) DiPEA and 653 mg (1.04 mmol, 50 wt % in ethyl acetate) T3P® at 0° C. The reaction mixture was allowed to warm up to room temperature overnight, concentrated to dryness and the residue was diluted in ethyl acetate. The organic layer was further washed with 1 M HCl, satd NaHCO₃, H₂O, brine, dried over Na₂SO₄ and concentrated in vacuo. Purification by flash chromatography (ethyl acetate) resulted in the isolation of 15a (93.0 mg, 0.13 mmol, 44%) as a white solid. R_{f}: 0.20 (ethyl acetate). [α]²⁰_{D}=+20.5° (c=1.0, CHCl₃). Mp: 98-100° C. ¹H NMR (400 MHz, CDCl₃): δ 0.21 (s, 6H), 0.97 (d, J=6.8 Hz, 3H), 1.02 (s, 9H), 1.64 (s, 3H), 2.21 (m, 2H), 2.33 (m, 4H), 2.68 (dd, J=16.6, 3.5 Hz, 1H), 2.81 (dd, J=16.6, 10.0 Hz, 1H), 2.94 (s, 3H), 3.21 (dd, J=15.8, 10.0 Hz, 1H), 3.36 (dd, J=15.6, 6.3 Hz, 1H), 3.96 (m, 1H), 4.02 (ddd, J=10.5, 10.5, 3.5 Hz, 1H), 4.54 (qd, J=6.5, 6.5 Hz, 1H), 5.09 (t, J=7.2 Hz, 1H), 5.25 (m, 1H), 5.55 (dd, J=10.0, 6.3 Hz, 1H), 6.38 (d, J=5.5 Hz, NH), 6.79 (d, J=8.3 Hz, 1H), 6.94 (s, 1H), 6.98 (dd, J=8.3, 2.3 Hz, 1H), 7.13 (m, 2H, NH), 7.19 (d, J=2.3 Hz, 1H), 7.32 (d, J=8.0 Hz, 1H), 7.59 (d, J=7.8 Hz, 1H), 8.07 (s, NH). ¹³C NMR (100 MHz, CDCl₃): δ −4.4, 16.1, 17.3, 18.3, 23.1, 25.6, 27.1, 30.7, 34.5, 35.7, 40.2, 46.0, 48.9, 56.3, 64.8, 110.9, 111.1, 118.5, 119.4, 120.6, 121.4, 122.1, 122.1, 125.4, 125.6, 127.3, 127.8, 135.1, 135.8, 136.1, 150.7, 169.5, 170.6, 171.8, 174.1. HRMS (Cl) m/z calc. for C₃₈H₅₂ClN₄O₆Si (M+H)⁺: 723.3339. found 723.3324. LCMS (Luna 3μ C18, 50×4.6 mm, H₂O:ACN 30:70, 20 min, rt, 0.6 mL/min): t_{R}=11.731 min @ m/z [+Na]⁺745.

### (4R,7R,10S,E)-4-(4-((tert-butyldimethylsilyl)oxy)-3-chlorophenyl)-7-((2-chloro-1H-indol-3-yl)methyl)-8,10,15-trimethyl-1-oxa-5,8,11-triazacyclooctadec-15-ene-2,6,9,12-tetraone (15b)

According to lactam 15a, double chlorinated lactam 15b was prepared from acid 14b (150 mg, 171 μmol), trifluoroacetic acid (0.70 mL, 9.09 mmol), DiPEA (0.12 mL, 0.69 mmol) and T3P® (377 mg, 599 μmol, 50 wt % in ethyl acetate). Purification by flash chromatography (ethyl acetate) gave rise to 15b (51 mg, 67.0 μmol, 39%) as a white solid. R_{f}: 0.36 (ethyl acetate). ¹H NMR (400 MHz, CDCl₃): δ 0.21 (s, 6H), 0.63 (d, J=6.8 Hz, 3H), 1.02 (s, 9H), 1.61 (s, 3H), 2.16 (m, 2H), 2.35 (m, 4H), 2.66 (m, 1H), 2.83 (dd, J=16.8, 10.5 Hz, 1H), 2.93 (s, 3H), 3.26 (m, 2H), 3.98 (m, 2H), 4.33 (m, 1H), 5.06 (t, J=7.2 Hz, 1H), 5.24 (ddd, J=10.3, 7.5, 3.0 Hz; 1H), 5.61 (dd, J=10.2, 6.2 Hz, 1H), 6.39 (s, NH), 6.79 (d, J=8.3 Hz, 1H), 7.01 (dd, J=8.5, 2.3 Hz, 1H), 7.07 (m, 2H), 7.17 (m, 1H, NH), 7.23 (d, J=2.3 Hz, 1H), 7.47 (d, J=7.8 Hz, 1H), 8.68 (s, NH). ¹³C NMR (100 MHz, CDCl₃): δ −4.4, 15.9, 16.6, 18.3, 21.9, 25.6, 27.1, 31.2, 34.4, 35.9, 40.2, 45.8, 49.1, 55.7, 64.8, 107.1, 110.5, 118.2, 120.1, 120.5, 121.4, 121.6, 122.2, 125.4, 125.6, 127.3, 127.8, 134.4, 135.2, 135.9, 150.7, 169.5, 170.6, 171.8, 173.9. HRMS (Cl) m/z calc. for C₃₈H₅₁Cl₂N₄O₆Si (M+H)⁺: 757.2949. found 757.2943.

### (4R,7R,10S,E)-7-((1H-indol-3-yl)methyl)-4-(3-bromo-4-((tert-butyldimethylsilyl)oxy)phenyl)-8,10,15-trimethyl-1-oxa-5,8,11-triazacyclooctadec-15-ene-2,6,9,12-tetraone (15c)

According to lactam 15a, brominated lactam 15b was prepared from acid 14c (205 mg, 231 μmol), trifluoroacetic acid (0.89 mL, 11.6 mmol), DiPEA (0.16 mL, 0.93 mmol) and T3P® (509 mg, 810 μmol, 50 wt % in ethyl acetate). Purification by flash chromatography (ethyl acetate) gave rise to 15c (93 mg, 121 μmol, 52%) as a white solid. R_{f}: 0.21 (ethyl acetate). [α]²⁰_{D}=+17.6° (c=1.0, CHCl₃). Mp: 88-90° C. ¹H NMR (400 MHz, CDCl₃): δ 0.23 (s, 6H), 0.97 (d, J=7.0 Hz, 3H), 1.03 (s, 9H), 1.64 (s, 3 H), 2.19 (m, 2H), 2.34 (m, 4H), 2.67 (dd, J=16.5, 3.3 Hz, 1H), 2.81 (dd, J=16.8, 10.3 Hz, 1H), 2.93 (s, 3H), 3.21 (dd, J=15.8, 10.0 Hz, 1H), 3.37 (dd, J=16.3, 6.0 Hz, 1H), 3.96 (m, 1H), 4.03 (ddd, J=10.5, 10.5, 3.0 Hz, 1H), 4.52 (m, 1H), 5.09 (t, J=7.0 Hz, 1H), 5.25 (ddd, J=10.5, 7.8, 3.3 Hz, 1H), 5.56 (dd, J=10.0, 6.3 Hz, 1H), 6.37 (d, J=5.8 Hz, NH), 6.77 (d, J=8.3 Hz, 1H), 6.95 (s, 1H), 7.03 (dd, J=8.4, 2.1 Hz, 1H), 7.13 (m, 2H, NH), 7.32 (m, 1H), 7.37 (d, J=2.0 Hz, 1H), 7.59 (d, J=7.8 Hz, 1H), 8.13 (s, NH). ¹³C NMR (100 MHz, CDCl₃): δ −4.3, 16.1, 17.3, 18.3, 23.1, 25.7, 27.1, 30.6, 34.5, 35.7, 40.2, 46.0, 48.8, 56.2, 64.8, 110.9, 111.1, 115.4, 118.5, 119.4, 120.0, 121.4, 122.0, 122.1, 126.2, 127.3, 130.9, 135.4, 138.8, 136.1, 151.8, 169.5, 170.6, 171.7, 174.0. HRMS (Cl) m/z calc. for C₃₈H₅₂BrN₄O₆Si (M)⁺: 766.2756. found 766.2741. LCMS (Luna 3μ C18, 50×4.6 mm, H₂O:ACN 30:70, 20 min, rt, 0.6 mL/min): t_{R}=12.655 min @ m/z [+Na]⁺789.

### (4R,7R,10S,E)-7-((1H-indol-3-yl)methyl)-4-(3-chloro-4-hydroxyphenyl)-8,10,15-trimethyl-1-oxa-5,8,11-triazacyclooctadec-15-ene-2,6,9,12-tetraone (16a)

To a solution of 72.0 mg (95.0 μmol) cyclodepsipeptide 15a in 1 mL dry THF was added 27.0 mg (104 μmol) TBAF trihydrat. After 1 h, ethyl acetate was added and the solution was washed with 1 M HCl, brine, dried over Na₂SO₄ and concentrated in vacuo. Purification by flash chromatography (ethyl acetate) gave rise to 16a (47.0 mg, 77.0 μmol, 81%) as a white solid. R_{f}: 0.16 (ethyl acetate). [α]²⁰_{D}=+4.0° (c=1.0, CHCl₃). Mp: 122-126° C. ¹H NMR (400 MHz, CDCl₃): δ 0.97 (s, 3H), 1.63 (s, 3H), 1.71 (s, OH), 2.30 (m, 6H), 2.64 (d, J=16.1, 1H), 2.86 (dd, J=16.9, 11.2 Hz, 1H), 2.94 (s, 3H), 3.19 (dd, J=15.8, 10.3 Hz, 1H), 3.42 (dd, J=15.8, 5.8 Hz, 1H), 3.93 (m, 1H), 4.05 (ddd, J=10.8, 10.8, 2.8 Hz, 1H), 4.45 (m, 1H), 5.08 (t, J=7.2 Hz, 1H), 5.19 (m, 1H), 5.63 (m, 1H), 6.27 (s, NH), 6.83 (s, 1H), 6.93 (m, 2H), 7.11 (m, 2H), 7.20 (d, J=2.0 Hz, 1H), 7.31 (d, J=8.0 Hz, 1H), 7.35 (s, NH), 7.58 (d, J=8.0 Hz, 1H), 8.17 (s, NH). ¹³C NMR (100 MHz, CDCl₃): δ 15.9, 16.7, 22.9, 27.1, 30.8, 34.4, 36.1, 40.3, 46.0, 49.3, 56.5, 64.7, 110.9, 111.1, 116.6, 118.5, 119.4, 120.1, 121.4, 122.0, 122.0, 125.6, 127.2, 127.4, 134.5, 136.1, 136.1, 151.1, 169.6, 170.6, 172.2, 174.2. HRMS (Cl) m/z calc. for C₃₂H₃₈ClN₄O₆(M)⁺: 609.2474. found 609.2474.

### (4R,7R,10S,E)-7-((2-chloro-1H-indol-3-yl)methyl)-4-(3-chloro-4-hydroxyphenyl)-8,10,15-trimethyl-1-oxa-5,8,11-triazacyclooctadec-15-ene-2,6,9,12-tetraone (16b)

According to chondramide-derivative 16a, double chlorinated derivative 16b was prepared from cyclodepsipeptide 15b (49 mg, 65.0 μmol) and TBAF trihydrat (24.0 mg, 78.0 μmol). Purification by flash chromatography (ethyl acetate) gave rise to 16b (38 mg, 59.0 μmol, 91%) as a white solid. R_{f}: 0.30 (ethyl acetate). [α]²⁰_{D}=+35.2° (c=1.0, CHCl₃). Mp: 135-139° C. ¹H NMR (400 MHz, CDCl₃): δ 0.71 (d, J=6.8 Hz, 3H), 1.62 (s, 3H), 1.70 (s, OH), 2.20 (m, 5H), 2.39 (m, 1H), 2.63 (dd, J=17.1, 3.0 Hz, 1H), 2.86 (dd, J=16.9, 10.9 Hz, 1H), 2.98 (s, 3H), 3.19 (dd, J=15.3, 11.0 Hz, 1H), 3.34 (dd, J=15.3, 5.5 Hz, 1H), 3.94 (m, 1H), 4.04 (ddd, J=10.5, 10.5, 2.8 Hz, 1H), 4.33 (m, 1H), 5.06 (t, J=7.2 Hz, 1H), 5.19 (ddd, J=10.0, 6.8, 2.8 Hz, 1H), 5.64 (dd, J=10.8, 5.3 Hz, 1H), 6.14 (s, J=5.3 Hz, NH), 6.83 (d, J=8.5 Hz, 1H), 6.99 (dd, J=8.4, 2.1 Hz, 1H), 7.06 (dd, J=6.9, 6.9 Hz, 1H), 7.12 (dd, J=7.0, 7.0 Hz, 1H), 7.20 (m, 2H), 7.29 (d, J=6.8 Hz, NH), 7.47 (d, J=7.5 Hz, 1H), 8.38 (s, NH). ¹³C NMR (100 MHz, CDCl₃): δ 15.9, 16.2, 21.9, 27.0, 31.4, 34.4, 36.3, 40.3, 45.8, 49.4, 55.9, 64.7, 107.2, 110.5, 116.6, 118.2, 120.1, 120.1, 121.4, 121.6, 122.3, 125.8, 127.1, 127.4, 134.4, 134.5, 136.1, 151.1, 169.6, 170.7, 172.2, 174.1. HRMS (Cl) m/z calc. for C₃₂H₃₇Cl₂N₄O₆(M)⁺: 643.2085. found 643.2098.

### (4R,7R,10S,E)-7-((1H-indol-3-yl)methyl)-4-(3-bromo-4-hydroxyphenyl)-8,10,15-trimethyl-1-oxa-5,8,11-triazacyclooctadec-15-ene-2,6,9,12-tetraone (16c)

According to chondramide-derivative 16a, brominated derivative 16c was prepared from cyclodepsipeptide 15c (73.0 mg, 95.0 μmol) and TBAF trihydrat (36.0 mg, 114 μmol). Purification by flash chromatography (ethyl acetate) gave rise to 16c (39.0 mg, 60.0 μmol, 63%) as a white solid. R_{f}: 0.18 (ethyl acetate). [α]²⁰_{D}=+7.8° (c=1.0, CHCl₃). Mp: 129-132° C. ¹H NMR (400 MHz, CDCl₃): δ 0.93 (d, J=7.0 Hz, 3H), 1.62 (s, 3H), 2.21 (m, 5H), 2.41 (m, 1H), 2.62 (dd, J=16.9, 2.9, 1H), 2.87 (dd, J=17.1, 11.0 Hz, 1H), 2.93 (s, 3H), 3.18 (dd, J=15.9, 10.4 Hz, 1H), 3.43 (dd, J=15.9, 5.7 Hz, 1H), 3.91 (m, 1H), 4.05 (dd, J=10.8, 10.8 Hz, 1H), 4.38 (m, 1H), 5.06 (t, J=7.3 Hz, 1H), 5.17 (m, 1H), 5.66 (dd, J=10.3, 5.8 Hz, 1H), 6.33 (d, J=4.8 Hz, NH), 6.78 (d, J=8.5 Hz, 1H), 6.89 (s, 1H), 6.93 (dd, J=8.5, 2.0 Hz, 1H), 7.07 (dd, J=7.4, 7.4 Hz, 1H), 7.13 (dd, J=7.2, 7.2 Hz, 1H), 7.29 (d, J=8.0 Hz, 1H), 7.36 (d, J=2.0 Hz, 1H), 7.46 (d, J=6.8 Hz, NH), 7.56 (d, J=7.8 Hz, 1H), 7.87 (s, OH), 8.31 (s, NH). ¹³C NMR (100 MHz, CDCl₃): δ 15.9, 16.4, 22.9, 27.0, 30.9, 34.3, 36.4, 40.3, 46.0, 49.4, 56.5, 64.6, 109.9, 110.8, 111.1, 116.4, 118.4, 119.2, 121.4, 121.9, 122.1, 126.1, 127.4, 130.4, 134.8, 136.1, 136.2, 152.3, 169.7, 170.7, 172.4, 174.4. HRMS (Cl) m/z calc. for C₃₂H₃₈BrN₄O₆(M)⁺: 653.1969. found 653.1951.

### Phosphorylations and Glycosylations

These modifications on the tyrosine unit can be performed according to standard protocols.^{[8,9,10,11]}

### LITERATURE

[1] Jiang W., Wanner J., Lee R. J., Bounaud P-Y., Boger D. L., J. Am. Chem. Soc., 2003, 125(7), 1877-1887.
[2] Prieto M., Mayor S., Rodriguez K., Williams P. L., Giralt E., J. Org. Chem., 2007, 72 (3), 1047-1050.
[3] Eggert U., Diestel R., Sasse F., Jansen R., Kunze B., Kalesse M., Angew. Chem., 2008, 120, 6578-6582.
[4] Hassfeld J., Eggert U., Kalesse M., Synthesis, 2005, 7, 1183-1199:
[5] Schmauder A., Müller S., Maier M. E., Tetrahedron, 2008, 64, 6263-6269.
[6] Ashworth P., Broadbelt B., Jankowski P., Kocienski P., Synthesis, 1995, 199.
[7] Schmauder A., Sibley L. D., Maier M. E., Chem. Eur. J., 2010, 4328.4336.
[8] Silverberg L. J., Dillon J. L., Vemishetti P., Tetrahedron Letters, 1996, 771-774.
[9] Jensen K. J., Meldal M., Bock K., J. Chem. Soc. Perkin Trans. 1, 1993, 2119-2129.
[10] Ernst B., Winkler T., Tetrahedron Letters, 1989, 3081-3084.
[11] Wadouachi A., Kovensky J., Molecules, 2011, 3933-3968.

### Reagents

All chemicals were of reagent grade quality and were obtained from commercial sources. Reference compounds chondramide A˜C were provided by the Microbial Drugs research group (MWIS) at the Helmholtz Centre for Infection Research (HZI, Braunschweig, Germany).

### Cell Cultures

Cell lines were obtained from the American Type Culture Collection (ATCC) and the German Collection of Microorganisms and Cell Cultures (Deutsche Sammlung für Mikroorganismen and Zellkulturen, DSMZ), and HUVEC (single donor) were purchased from PromoCell. Cells were cultured under the conditions recommended by the respective depositor in a standard humidified incubator at 37° C. with 5% CO₂.

### Growth Inhibition Experiments

Cells were seeded at 6×10³ cells per well of 96-well plates (Corning CellBind®) in 180 μl complete medium and directly treated with chondramides dissolved in methanol in a serial dilution. Each compound was tested in duplicate for 5 d, as well as the internal methanol control. After 5 d incubation, 20 μl of 5 mg/ml MTT (Mosmann, T. (1983). Rapid Colorimetric Assay for Cellular Growth and Survival: Application to Proliferation and Cytotoxicity Assays. J. Immunol. Meth. 65, 55-63) in PBS was added per well and it was further incubated for 2 h at 37° C. The medium was then discarded and cells were washed with 100 μl PBS before adding 100 μl 2-propanol/10 N HCl (250:1) in order to dissolve formazan granules. The absorbance at 570 nm was measured using a microplate reader (EL808, Bio-Tek Instruments Inc.), and cell viability was expressed as percentage relative to the respective methanol control. GI₅₀ values were obtained by sigmoidal curve fitting. Testing of fractions was performed likewise using 1.2×10⁴ cells per well. Samples were dissolved in cell culture medium and cells were treated for 2 d before adding MTT. Viability was expressed relative to the untreated control.

### HCS Assay on Lysosomal Integrity

Human U-2 OS osteosarcoma cells were seeded at 5×10³ cells per well in 96-well imaging plates (BD Falcon). After o/n equilibration, the cells were treated for 4 h with samples at assigned concentrations. For labeling, cells were washed twice with PBS, and 10 μg/ml Hoechst33342 (Molecular Probes) and 5 μg/ml acridine orange (Sigma) dissolved in PBS was added for 15 min. After washing, the samples were imaged at 200-fold magnification on an automated microscope (BD Pathway855) suitable for high-content screening with appropriate filter sets for Hoechst and acridine orange (green/red) fluorescence.

### Segmentation and Parameter Selection (Lysosomal Integrity)

Images for high-content analysis were recorded in the red and green channel for acridine orange fluorescence and Hoechst channel for nuclei. In living cells, the weak base acridine orange accumulates in acidic compartments, mainly lysosomes, exhibiting a red fluorescence. At lower concentrations of the dye in the whole cell, fluorescence is green. By this, images in the green and Hoechst channel were used for segmentation with a cyto-nuc-dual mask in AttoVision v1.6.2 software (BD Biosciences), which results in segments (ROIs, regions of interest) for both, nuclei and cytoplasm. Upon disruption of lysosomal integrity, the pH increases (less acidic) and the red fluorescence of acridine orange disappears mostly. For analysis of lysosomal pH, the intensity value of green fluorescence (background) was divided by the red fluorescence intensity in the cytoplasmic segment. The latter value was calculated after an overlay of the cytoplasmic ROIs from the green channel in the red channel. Parameters were calculated within a well on a single-cell basis within the cytoplasmic ROIs (regions of interest) and expressed as average.

### HCS Assay on Actin Filaments

Human U-2 OS cells were seeded as described for assays on lysosomal integrity. After o/n cultivation, cells were treated with samples at assigned concentrations for 1-2 d. For immunostaining or labeling with ActiStain 555 phalloidin (Cytoskeleton, Inc.) cells were fixed with cold (−20° C.) acetone/MeOH (1:1) for 10 min or 3.7% paraformaldehyde in PBS at r.t., respectively. After washing with PBS, the cells were permeabilized with 0.01% (0.5% for ActiStain 555 phalloidin) Triton-X 100 in PBS. For immunostaining, U-2 OS cells were probed with the following antibody combination: β-actin mAb (1:2000, Sigma)/goat-anti-mouse-Alexa 488 (1:1000, Molecular Probes), diluted in PBS/10% FBS. Cells were incubated with primary antibody for 45 min at 37° C., followed by incubation with the secondary antibody under the same conditions. Actin labeling with ActiStain 555 phalloidin (100 nM in PBS) was done at r.t. for 30 min. For both preparations nuclei were stained with Hoechst 33342 (5 μg/ml; Molecular Probes), and a whole cell stain was added to immunostained cells (1 μg/ml; HCS CellMask™ Red stain, Invitrogen). The samples were imaged on an automated microscope (BD Pathway 855) with appropriate filter sets for Alexa 488, Hoechst, TRITC, and CellMask™ Red fluorescence.

### Segmentation and Parameter Selection (Actin Disruption)

As depicted in Figure S2 cytoplasmic segments were obtained by means of whole cell staining with HCS CellMask Red™ (Invitrogen) and nuclear staining with Hoechst33342 (Molecular Probes) for cyto-nuc-dual segmentation in AttoVision v1.6.2 (BD Biosciences). Several measures for actin polymerization events were determined. In an unbiased approach, an experimental test set was created and samples were classified into five cellular states. Important features for description of actin filament disruption were defined by statistical methods, like cross-validation by the lasso method (unpublished results). The standard deviation of actin fluorescence was amongst the optimal features and was chosen for the quantification of HCS assays on actin. In Bravais-Pearson correlation models this parameter also correlated well with, in the context of actin polymerization, biological meaningful features, like the intensity of nuclear fluorescence. The actin fluorescence SD was calculated within a well on a single-cell basis within the cytoplasmic ROIs (regions of interest) and expressed as average.

### Actin Polymerization Assay.

Fluorescence-based actin polymerization assays were performed using pyrene muscle actin (Cytoskeleton, Inc.) according to the supplier's protocol. Chondramides were dissolved in non-polymerizing buffer (5 mM Tris-HCl pH 8.0, 0.2 mM CaCl₂, 0.2 mM ATP, 1 mM DTT) and mixed with pyrene actin diluted in the same buffer to give a final concentration of 20 μM chondramide and 5 μM actin. Samples were prepared in black, low-volume 384-well plates (Corning, NBS™ surface). The fluorescence at 405 nm (excitation at 360 nm) was scanned every 30 s for 85 min at 37° C. on a microplate reader (Spectramax M5^{e}, Molecular Devices). Experiments were performed in duplicates and the average relative fluorescence of polymerizing pyrene actin is given. Slopes were calculated in the linear range of kinetic measurements.

### Semi-Preparative HPLC

Measurements were conducted on a Dionex HPLC system (Famos autosampler, P680 pump, TCC100 thermostat, and PDA100 detector), equipped with a Phenomenex Luna C18, 250×4.6 mm, 5 μm dp column. Separation was achieved by variable linear gradients using (A) H₂O and (B) ACN at a flow rate of 2.5 ml/min and 25° C. The gradient started at 5-60% B with a 0.5 min hold and increased to 100% B in a variable time frame. UV data was acquired at 220 and 280 nm. The sample was injected by μl-pick-up technology with a water/ACN (50:50 v/v) mixture as supporting solvent. A maximum of 75 μl sample was injected before manual fraction collection.

### High-Resolution Mass Spectrometry

All measurements were performed on a Dionex Ultimate 3000 RSLC system using a Waters BEH C18, 100×2.1 mm, 1.7 μm dp column by injection of 2 μl methanolic sample. Separation was achieved by a linear gradient with (A) H₂O+0.1% FA to (B) ACN+0.1% FA at a flow rate of 550 μl/min and 45° C. The gradient was initiated by a 0.39 min isocratic step at 5% B, followed by an increase to 95% B in 18 min to end up with a 1.5 min flush step at 95% B before reequilibration with initial conditions. UV and MS detection were performed simultaneously. Coupling the HPLC to a MS was supported by an Advion Triversa Nanomate nano-ESI system attached to a Thermo Fisher Orbitrap. Mass spectra were acquired in centroid mode ranging from 200-2000 m/z at a resolution of R=30000.

### NMR

Data was acquired on a DRX 500 MHz and a Bruker Ascend 700 MHz spectrometer, the latter one equipped with a CryoProbe system (Bruker Biospin GmbH, Germany). More detailed information can be found in Supplemental Experimental Procedures.

### Nomenclature

The nomenclature of the chondramides is based on original chondramides A˜D. Since most variations occur on R¹ this position was chosen to determine the letter of the according chondramide. Since A was already mentioned in literature A was set to Al. B has no variation on R¹ in comparison to A therefore it was set to A2 in the nomenclature used herein. C has two protons on position R¹ and was therefore chosen as C1. Chondramide D has no variation in R¹ compared to C but the same substitution pattern than B in comparison to chondramide A, therefore D was set to C2 in the nomenclature used herein. The further numbers are based on the substitution pattern meaning that e.g. A5, C5 and E5 have the same substitution pattern except on position R¹. Since the letters A to D are already in use, it has been decided to name a further variation on position R¹ (—OH) as E with its according numbers.

### NMR

The structure of all listed chondramides was established by analysis of 1H, DQF-COSY, HSQC, and HMBC data. This data was measured on a DRX 500 MHz and a Bruker Ascend 700 MHz spectrometer equipped with a CryoProbe system (Bruker Biospin GmbH, Germany).

To establish the stereochemistry homonuclear coupling constants of ¹H, chemical shifts of ¹H and ¹³C of the original chondramide A, the synthesized chondramide A and chondramide A from SBCm007 was compared as well as retention times of a HPLC run of the original chondramide A and the one from SBCm007, carried out with a gradient from 5% to 95% ACN and Water both supplemented with 0.1% FA, as done in the total synthesis paper on chondramide A (Schmauder, A., Sibley, L. D., and Maier, M. E. (2010). Total Synthesis and Configurational Assignment of Chondramide A. Chem. Eur. J. 16, 4328-4336). Due to the reported biosynthesis of chondramides (Rachid S., Krug D., Kunze B., Kochems I., Scharfe M., Zabriskie T., Blocker H., Müller R., Chemistry & Biology, 2006 (14), 667-681) the stereochemistry of the chondramide core structure can be considered as being identical in all natural derivatives.

### Chondramide Derivatives

All derivatives were either structure elucidated or proposed by HR-MS data dereplication (NMR: structure elucidated; n.d.: not detected); Residues refer to formula (II).

| | | | | | | m/z | m/z |
|---|---|---|---|---|---|---|---|
| | | | | | | [M + H]⁺ | [M + H]⁺ |
| | R¹ | R² | R³ | R⁴ | R⁵ | theor. | found |
| Chondramide A (A1) | OMe | H | OH | H | Me | 647.3445 | NMR |
| Chondramide B (A2) | OMe | Cl | OH | H | Me | 681.3056 | 681.3049 |
| Chondramide A3 | OMe | H | OH | Cl | Me | 681.3056 | NMR |
| Chondramide A4 | OMe | Cl | OH | Cl | Me | 715.2666 | NMR |
| Chondramide A5 | OMe | H | P | H | Me | 727.3109 | 727.3102 |
| Chondramide A6 | OMe | Cl | P | H | Me | 761.2719 | NMR |
| Chondramide A7 | OMe | H | P | Cl | Me | 761.2719 | 761.2713 |
| Chondramide A8 | OMe | Cl | P | Cl | Me | 795.2329 | NMR |
| Chondramide A9 | OMe | H | C₆H₉O₇ | Cl | Me | 857.3376 | NMR |
| Chondramide A10 | OMe | Cl | C₆H₉O₇ | Cl | Me | 891.2987 | NMR |
| Chondramide C (C1) | H | H | OH | H | Me | 617.334 | 617.3333 |
| Chondramide D (C2) | H | Cl | OH | H | Me | 651.295 | 651.2944 |
| Chondramide C3 | H | H | OH | Cl | Me | 651.295 | 651.2944 |
| Chondramide C4 | H | Cl | OH | Cl | Me | 685.256 | 685.2554 |
| Chondramide C5 | H | H | P | H | Me | 697.3003 | 685.2554 |
| Chondramide C6 | H | Cl | P | H | Me | 731.2613 | 731.2607 |
| Chondramide C7 | H | H | P | Cl | Me | 731.2613 | 731.2607 |
| Chondramide C8 | H | Cl | P | Cl | Me | 765.2224 | n.d. |
| Chondramide C9 | H | H | C₆H₉O₇ | Cl | Me | 827.3271 | 827.3265 |
| Chondramide C10 | H | Cl | C₆H₉O₇ | Cl | Me | 861.2881 | n.d. |
| Chondramide E1 | OH | H | OH | H | Me | 633.3289 | 633.3283 |
| Chondramide E2 | OH | Cl | OH | H | Me | 667.2899 | NMR |
| Chondramide E3 | OH | H | OH | Cl | Me | 667.2899 | 667.2893 |
| Chondramide E4 | OH | Cl | OH | Cl | Me | 701.2509 | 701.2503 |
| Chondramide E5 | OH | H | P | H | Me | 713.2952 | 713.2946 |
| Chondramide E6 | OH | Cl | P | H | Me | 747.2562 | 747.2556 |
| Chondramide E7 | OH | H | P | Cl | Me | 747.2562 | 747.2556 |
| Chondramide E8 | OH | Cl | P | Cl | Me | 781.2173 | n.d. |
| Chondramide E9 | OH | H | C₆H₉O₇ | Cl | Me | 843.322 | n.d. |
| Chondramide E10 | OH | Cl | C₆H₉O₇ | Cl | Me | 877.283 | n.d. |
| Chondramide A3 (linear) | OMe | H | OH | Cl | Me | 699.3161 | NMR |
| Bromo-Chondramide A3 | OMe | H | OH | Br | Me | 725.255 | NMR |
| Chondramide A9 Variant | OMe | H | C₁₅H₂₄NO₁₂ | Cl | Me | 1074.4327 | 1074.432 |
| Bromo-Chondramide A9 | OMe | H | C₆H₉O₇ | Br | Me | 901.2871 | 901.2865 |
| Chondramide A10 Variant | OMe | Cl | C₁₅H₂₄NO₁₂ | Cl | Me | 1108.3937 | NMR |
| Propionyl-Chondramide C1 | H | H | OH | H | Et | 631.3496 | NMR |
| Bromo-Chondramide C3 | H | H | OH | Br | Me | 695.2445 | NMR |
| Propionyl-Bromo- | H | H | OH | Br | Et | 709.2601 | NMR |
| Chondramide C3 | | | | | | | |
| Bromo-Chondramide C9 | H | H | C₆H₉O₇ | Br | Me | 871.2766 | 871.2759 |

### NMR Tables

Synthesized Chondramide A(1) and Chondramide A from SBcm007

Chondramide A
(700 MHz, 175 MHz, MeOD)

| Cmc5/synthesized | | | | SBcm007 | | | |
|---|---|---|---|---|---|---|---|
| | 13C | 1H | J(in Hertz) | | 13C | 1H | J(in Hertz) |

PKS

| | | | | | | | |
|---|---|---|---|---|---|---|---|
| 1 | 176.9 | x | | 1 | 176.5 | x | |
| 2 | 40.2 | 2.59-2.72 | m | 2 | 40.2 | 2.63-2.69 | m |
| 2-Me | 19.1 (3H) | 1.08 | d 6.80 | 2-Me | 19.1 | 1.09 | d 6.78 |
| 3 | 46.0 (2H) | 2.23; 2.03 | dd 13.0, 12.6; | 3 | 46.0 | 2.24; 2.04 | dd 12.6, 12,6; |
| | | | dd 12.6 2.6 | | | | 13.6, 3.31 |
| 4 | 134.7 | x | | 4 | 134.3 | x | |
| 4-Me | 16.0 (3H) | 1.68 | s | 4-Me | 15.9 | 1.69 | s |
| 5 | 129.1 | 4.81-4.87 | m | 5 | 129.1 | 4.85 | Waterpeak |
| 6 | 38.7 | 2.45-2.57 | m | 6 | 38.7 | 2.47-2.55 | m |
| 6-Me | 17.9 (3H) | 0.91 | d 6.60 | 6-Me | 17.9 | 0.92 | d 6.72 |
| 7 | 79.2 | 4.47-4.56 | m | 7 | 79.2 | 4.51.4.56 | m |
| 7-Me | 18.9 (3H) | 0.86 | d 6.1 | 7-Me | 18.9 | 0.87 | d 6.24 |

Tyr Deriv.

| | | | | | | | |
|---|---|---|---|---|---|---|---|
| 1′ | 173.5 | x | | 1′ | 173.0 | x | |
| 2′ | 83.4 | 3.85 | d 10.1 | 2′ | 83.5 | 3.86 | d 9.89 |
| O—Me′ | 58.3 (3H) | 3.14 | s | O—Me′ | 58.3 | 3.15 | s |
| 3′ | 55.7 | 5.03 | d 9.9 | 3′ | 55.7 | 5.04 | dd 9.82; 9.67 |
| 4′ | 131.3 | x | | 4′ | 130.9 | x | |
| 5′ | 129.5 | 6.99 | d 8.6 | 5′ | 129.6 | 6.99 | d 8.69 |
| 6′ | 116.1 | 6.67 | d 8.6 | 6′ | 116.1 | 6.68 | d 8.60 |
| 7′ | 157.9 | x | | 7′ | 157.5 | x | |

N—Me-Trp

| | | | | | | | |
|---|---|---|---|---|---|---|---|
| 1″ | 171.3 | | | 1″ | 170.9 | x | |
| 2″ | 56.9 | 5.52 | dd 8.1, 8.1 | 2″ | 56.9 | 5.53 | dd 8.6, 8.6 |
| 3″ | 26.4 (2H) | 3.02 | d 8.1 | 3″ | 26.5 | 3.03 | dd 8.1, 1.9 |
| 4″ | 110.1 | x | | 4″ | 109.8 | x | |
| 5″ | 124.4 | 6.83 | s | 5″ | 124.4 | 6.84 | s |
| 6″ | 128.5 | x | | 6″ | 128.1 | x | |
| 7″ | 137.9 | x | | 7″ | 137.6 | x | |
| 8″ | 112.2 | 7.26 | d 8.1 | 8″ | 112.2 | 7.27 | d 8.10 |
| 9″ | 122.3 | 7.02-7.09 | m | 9″ | 122.3 | 7.06 | dd 7.52 |
| 10″ | 119.6 | 6.95-7.02 | m | 10″ | 119.6 | 6.98 | dd 7.45 |
| 11″ | 119.4 | 7.57 | d 7.8 | 11″ | 119.4 | 7.57 | d 7.88 |
| N—Me″ | 30.9 (3H) | 3.08 | s | N—Me″ | 30.9 | 3.09 | s |

Ala

| | | | | | | | |
|---|---|---|---|---|---|---|---|
| 1′″ | 174.9 | x | | 1′″ | 174.5 | x | |
| 2′″ | 45.9 | 4.73-4.81 | | 2′″ | 46.1 | 4.76-4.81 | m |
| 3′″ | 18.4 (3H) | 0.8 | d 7.1 | 3′″ | 18.4 | 0.81 | d 6.97 |

Chondramide A3 (700 MHz, 175 MHz, MeOD)
Cm7

| | 13C | 1H | J(in Hertz) |
|---|---|---|---|
| PKS | | | |
| 1 | 176.5 | x | |
| 2 | 39.9 | 2.64-2.70 | m |
| 2-Me | 18.8 | 1.09 | d 6.74 |
| | (3H) | | |
| 3 | 45.8 | 2.24; 2.05 | dd 12.90, 12.90; dd 13.33, |
| | (2H) | | 3.02 |
| 4 | 134.4 | x | |
| 4-Me | 15.7 | 1.7 | s |
| | (3H) | | |
| 5 | 128.8 | 4.85 | m, waterpeak |
| 6 | 38.3 | 2.48-2.54 | m |
| 6-Me | 17.5 | 0.92 | d 6.71 |
| | (3H) | | |
| 7 | 78.9 | 4.51-4.56 | m |
| 7-Me | 18.2 | 0.85 | d 7.00 |
| | (3H) | | |
| Tyr Deriv. | | | |
| 1′ | 172.9 | x | |
| 2′ | 82.9 | 3.82 | d 9.93 |
| O-Me′ | 57.9 | 3.16 | s |
| | (3H) | | |
| 3′ | 55.2 | 4.99 | dd 9.97, 9.47 |
| 4′ | 132.4 | x | |
| 5′ | 129.6 | 7.15 | d 2.11 |
| 6′ | 121.0 | x | |
| 7′ | 153.4 | x | |
| 8′ | 116.9 | 6.77 | d 8.38 |
| 9′ | 127.6 | 6.86 | dd 8.38, 2.08 |
| N-Me-Trp | | | |
| 1″ | 170.9 | | |
| 2″ | 56.7 | 5.51 | dd 8.37, 7.78 |
| 3″ | 26.2 | 3.07; 3.01 | dd 15.67, 7.53; dd 14.54, |
| | (2H) | | 8.64 |
| 4″ | 109.8 | x | |
| 5″ | 123.9 | 6.85 | s |
| 6″ | 128.2 | x | |
| 7″ | 137.6 | x | |
| 8″ | 111.9 | 7.26 | d 8.09 |
| 9″ | 122.0 | 7.05 | dd 7.91, 6.84 |
| 10″ | 119.3 | 6.98 | dd 7.92, 7.14 |
| 11″ | 119.0 | 7.57 | d 7.84 |
| N-Me″ | 30.5 | 3.09 | s |
| | (3H) | | |
| Ala | | | |
| 1″′ | 174.4 | x | |
| 2″′ | 45.7 | 4.79 | m, waterpeak |
| 3″′ | 18.6 | 0.88 | d 6.23 |
| | (3H) | | |

Chondramide A3 linear (700 MHz, 175 MHz, MeOD)
Cm7

| | 13C | 1H | J (in Hertz) |
|---|---|---|---|
| PKS | | | |
| 1 | 178.3 | x | |
| 2 | 39.3 | 2.49-2.55 | m |
| 2-Me | 16.9 | 1.02 | d 6.88 |
| | (3H) | | |
| 3 | 44.8 | 2.28-2.33; | m; dd 13.32, 8.63 |
| | (2H) | 1.95 | |
| 4 | 133.2 | x | |
| 4-Me | 16.1 | 1.62 | s |
| | (3H) | | |
| 5 | 131.2 | 4.99 | d 9.59 |
| 6 | 41.2 | 2.29-2.35 | m |
| 6-Me | 17.1 | 0.95 | d 6.72 |
| | (3H) | | |
| 7 | 72.7 | 3.41-3.47 | m |
| 7-Me | 20.9 | 1.06 | d 6.30 |
| | (3H) | | |
| Tyr Deriv. | | | |
| 1′ | 173.5 | x | |
| 2′ | 83.1 | 4.13 | d 6.28 |
| O—Me′ | 59.2 | 3.36 | s |
| | (3H) | | |
| 3′ | 54.8 | 5.25 | d 6.61 |
| 4′ | 130.5 | x | |
| 5′ | 130.4 | 7.32 | d 2.08 |
| 6′ | 120.5 | x | |
| 7′ | 153.4 | x | |
| 8′ | 115.8 | 6.8 | d 8.37 |
| 9′ | 128.8 | 7.1 | dd 8.39, 2.18 |
| N—Me-Trp | | | |
| 1″ | 171.8 | | |
| 2″ | 58.2 | 5.57 | dd 10.73, 5.69 |
| 3″ | 24.8 | 3.36-3.41; | m; dd 15.02, |
| | (2H) | 3.18 | 10.57 |
| 4″ | 110.2 | x | |
| 5″ | 123.3 | 7.02 | s |
| 6″ | 127.3 | x | |
| 7″ | 137.4 | x | |
| 8″ | 111.8 | 7.29 | d 8.12 |
| 9″ | 122.8 | 7.07 | dd 8.18, 7.29 |
| 10″ | 119.7 | 6.98 | dd 7.95, 7.07 |
| 11″ | 118.9 | 7.57 | d 8.00 |
| N—Me″ | 31.6 | 3.04 | s |
| | (3H) | | |
| Ala | | | |
| 1′″ | 175.3 | x | |
| 2′″ | 46.6 | 4.53 | q 6.61 |
| 3′″ | 16.1 | 0.8 | d 7.00 |
| | (3H) | | |

Chondramide A4 (700 MHz, 175 MHz, MeOD)
Cm7

| | 13C | 1H | J (in Hertz) |
|---|---|---|---|
| PKS | | | |
| 1 | 176.4 | x | |
| 2 | 39.8 | 2.60-2.67 | m |
| 2-Me | 18.8 | 1.07 | d 6.78 |
| | (3H) | | |
| 3 | 45.8 | 2.22; | dd 13.08, 12.2; dd 13.4, |
| | (2H) | 2.03 | 3.44 |
| 4 | 134.4 | x | |
| 4-Me | 15.7 | 1.68 | s |
| | (3H) | | |
| 5 | 128.8 | 4.83 | d 9.97 |
| 6 | 38.4 | 2.48-2.54 | m |
| 6-Me | 17.6 | 0.93 | d 6.74 |
| | (3H) | | |
| 7 | 79.0 | 4.52 | dq 8.09, 6.31 |
| 7-Me | 18.8 | 0.84 | d 6.23 |
| | (3H) | | |
| Tyr Deriv. | | | |
| 1′ | 172.9 | x | |
| 2′ | 82.9 | 3.85 | d 10.02 |
| O—Me′ | 57.9 | 3.16 | s |
| | (3H) | | |
| 3′ | 55.2 | 4.99 | d 10.02 |
| 4′ | 132.5 | x | |
| 5′ | 129.7 | 7.16 | d 2.15 |
| 6′ | 121.0 | x | |
| 7′ | 153.4 | x | |
| 8′ | 117.1 | 6.77 | d 8.36 |
| 9′ | 127.7 | 6.92 | dd 8.42, 2.17 |
| N—Me-Trp | | | |
| 1″ | 170.5 | | |
| 2″ | 55.5 | 5.58 | dd 8.93, 7.06 |
| 3″ | 25.0 | 2.96-3.05 | m |
| | (2H) | | |
| 4″ | 106.2 | x | |
| 5″ | 122.9 | x | |
| 6″ | 128.2 | x | |
| 7″ | 135.9 | x | |
| 8″ | 111.4 | 7.17 | d 8.09 |
| 9″ | 122.5 | 7.06 | ddd 8.19, 7.20, 1.13 |
| 10″ | 120.2 | 6.99 | ddd 8.03, 7.29, 0.97 |
| 11″ | 118.9 | 7.49 | d 7.91 |
| N—Me″ | 30.9 | 3.13 | s |
| | (3H) | | |
| Ala | | | |
| 1′″ | 174.5 | x | |
| 2′″ | 45.5 | 4.76 | q 7.07 |
| 3′″ | 17.9 | 0.75 | d 6.98 |
| | (3H) | | |

Chondramide A6 (700 MHz, 175 MHz, MeOD)
Cm7

| | 13C | 1H | J (in Hertz) |
|---|---|---|---|
| PKS | | | |
| 1 | 176.3 | x | |
| 2 | 39.8 | 2.65-2.70 | m |
| 2-Me | 18.3 | 1.09 | d 6.74 |
| | (3H) | | |
| 3 | 46.1 | 2.22; 2.07 | dd 13.01, 12.45; dd 13.24, |
| | (2H) | | 3.17 |
| 4 | 134.5 | x | |
| 4-Me | 15.5 | 1.7 | s |
| | (3H) | | |
| 5 | 128.8 | 4.83 | m, waterpeak |
| 6 | 38.5 | 2.48-2.53 | m |
| 6-Me | 18.0 | 0.93 | d 6.74 |
| | (3H) | | |
| 7 | 79.0 | 4.48-4.51 | m |
| 7-Me | 18.8 | 0.83 | d 6.29 |
| | (3H) | | |
| Tyr Deriv. | | | |
| 1′ | 172.9 | x | |
| 2′ | 82.8 | 3.83 | d 10.29 |
| O—Me | 57.9 | 3.09 | s |
| | (3H) | | |
| 3′ | 55.6 | 5 | d 10.37 |
| 4′ | 134.8 | x | |
| 5′ | 128.9 | 6.97 | d 8.06 |
| 6′ | 121.1 | 7.07-7.10 | m |
| 7′ | 153.3 | x | |
| N—Me-Trp | | | |
| 1″ | 169.9 | | |
| 2″ | 55.3 | 5.54 | dd 8.23, 7.73 |
| 3″ | 25.5 | 3.06; 2.85 | dd 14.33, 9.13; dd 14.50, 7.41 |
| | (2H) | | |
| 4″ | 105.3 | x | |
| 5″ | 123.0 | x | |
| 6″ | 127.9 | x | |
| 7″ | 135.8 | x | |
| 8″ | 111.9 | 7.3 | d 8.14 |
| 9″ | 122.4 | 7.07-7.10 | m |
| 10″ | 119.9 | 7.02 | dd 7.73, 7.20 |
| 11″ | 118.7 | 7.55 | d 7.76 |
| N—Me″ | 30.9 | 3.17 | s |
| | (3H) | | |
| Ala | | | |
| 1′″ | 174.4 | x | |
| 2′″ | 45.4 | 4.82 | m, waterpeak |
| 3′″ | 18.0 | 0.93 | d 6.63 |
| | (3H) | | |

Chondramide A8 (700 MHz, 175 MHz, MeOD)
Cm7

| | 13C | 1H | J (in Hertz) |
|---|---|---|---|
| PKS | | | |
| 1 | 176.7 | x | |
| 2 | 39.8 | 2.66-2.72 | m |
| 2-Me | 18.5 | 1.1 | d 6.70 |
| 3 | 46.1 | 2.22; 2.08 | dd 13.0, 2.6; dd 12.6 2.6 |
| | (2H) | | |
| 4 | 134.4 | x | |
| 4-Me | 15.5 | 1.7 | s |
| | (3H) | | |
| 5 | 128.8 | 4.83 | m, Waterpeak |
| 6 | 38.4 | 2.48-2.54 | m |
| 6-Me | 17.9 | 0.92 | d 6.74 |
| | (3H) | | |
| 7 | 79.3 | 4.48-4.53 | m |
| 7-Me | 18.9 | 0.84 | d 6.23 |
| | (3H) | | |
| Tyr Deriv. | | | |
| 1′ | 173.0 | x | |
| 2′ | 82.4 | 3.81 | d 10.3 |
| O—Me′ | 57.8 | 3.11 | s |
| | (3H) | | |
| 3′ | 55.2 | 4.98 | dd 9.90, 9.87 |
| 4′ | 137.7 | x | |
| 5′ | 130.4 | 7.19 | d 1.55 |
| 6′ | 126.4 | x | |
| 7′ | 148.3 | x | |
| 8′ | 122.3 | 7.29 | d 8.48 |
| 9′ | 127.3 | 6.88 | dd 8.35, 1.91 |
| N—H | X | 8.47 | d 9.53 |
| N—Me-Trp | | | |
| 1″ | 170.4 | | |
| 2″ | 55.4 | 5.52 | dd 8.50, 7.85 |
| 3″ | 25.6 | 3.11, 2.84 | m, dd 13.98, 7.10 |
| | (2H) | | |
| 4″ | 105.5 | x | |
| 5″ | 122.8 | x | |
| 6″ | 128.0 | x | |
| 7″ | 136.1 | x | |
| 8″ | 111.7 | 7.26 | d 8.11 |
| 9″ | 122.5 | 7.08 | ddd 8.86, 8.11, 0.99 |
| 10″ | 120.1 | 7.01 | ddd 8.32, 7.79, 0.90 |
| 11″ | 118.7 | 7.53 | d 7.85 |
| N—Me″ | 30.9 | 3.18 | s |
| | (3H) | | |
| Ala | | | |
| 1′″ | 174.5 | x | |
| 2′″ | 45.6 | 4.83 | m, Waterpeak |
| 3′″ | 18.2 | 0.96 | d 7.02 |
| | (3H) | | |

Chondramide A9 (500 MHz, 125 MHz, MeOD)
Cm7

| | 13C | 1H | J (in Hertz) |
|---|---|---|---|
| PKS | | | |
| 1 | 176.5 | x | |
| 2 | 39.9 | 2.67-2.76 | m |
| 2-Me | 18.7 | 1.11 | d 6.76 |
| | (3H) | | |
| 3 | 45.9 | 2.25; 2.08 | dd 12.76, 12.76; dd 13.09, |
| | (2H) | | 3.36 |
| 4 | 134.4 | x | |
| 4-Me | 15.6 | 1.72 | s |
| | (3H) | | |
| 5 | 128.8 | 4.86 | m, Waterpeak |
| 6 | 38.4 | 2.47-2.56 | m |
| 6-Me | 17.8 | 0.92 | d 6.68 |
| | (3H) | | |
| 7 | 79.2 | 4.48-4.54 | m |
| 7-Me | 18.8 | 0.87 | d 6.18 |
| | (3H) | | |
| Tyr Deriv. | | | |
| 1′ | 172.9 | x | |
| 2′ | 82.7 | 3.75 | d 10.19 |
| O—Me′ | 57.9 | 3.11 | s |
| | (3H) | | |
| 3′ | 55.2 | 4.93 | d 10.22 |
| 4′ | 135.3 | x | |
| 5′ | 130.5 | 7.14 | d 2.09 |
| 6′ | 123.5 | x | |
| 7′ | 153.1 | x | |
| 8′ | 117.0 | 7.03 | d 8.65 |
| 9′ | 126.9 | 6.73 | dd 8.58, 2.02 |
| N—Me-Trp | | | |
| 1″ | 170.7 | | |
| 2″ | 55.6 | 5.48 | dd 9.23, 6.74 |
| 3″ | 26.5 | 3.17; 2.85 | m 3.09-3.21; dd 13.95, 6.97 |
| | (2H) | | |
| 4″ | 109.4 | x | |
| 5″ | 123.9 | 6.69 | s |
| 6″ | 128.0 | x | |
| 7″ | 137.5 | x | |
| 8″ | 112.3 | 7.3 | d 8.12 |
| 9″ | 122.1 | 7.07 | dd 8.55, 7.23 |
| 10″ | 119.2 | 6.98 | dd 8.37, 7.31 |
| 11″ | 119.0 | 7.59 | d 7.84 |
| Ala | | | |
| 1′″ | 174.4 | x | |
| 2′″ | 45.7 | 4.86 | m, Waterpeak |
| 3′″ | 18.3 | 1.04 | d 6.95 |
| | (3H) | | |
| β-D-glucuronic | | | |
| acid | | | |
| 1 | 101.9 | 5.06 | d 7.63 |
| 2 | 74.3 | 3.62 | dd 8.86, 7.63 |
| 3 | 77.0 | 3.55 | dd 9.05, 9.05 |
| 4 | 72.9 | 3.65 | dd 9.19, 9.16 |
| 5 | 76.2 | 3.99 | d 9.72 |
| 6 | 173.3 | x | |

Chondramide A10 (500 MHz, 125 MHz, MeOD)
Cm7

| | 13C | 1H | J (in Hertz) |
|---|---|---|---|
| PKS | | | |
| 1 | 176.7 | x | |
| 2 | 39.9 | 2.64-2.72 | m |
| 2-Me | 18.8 | 1.09 | d 6.72 |
| | (3H) | | |
| 3 | 46.1 | 2.23; 2.06 | dd 12.89, 12.44; dd 13.34, |
| | (2H) | | 3.34 |
| 4 | 134.5 | x | |
| 4-Me | 15.5 | 1.7 | s |
| | (3H) | | |
| 5 | 128.7 | 4.83 | m, Waterpeak |
| 6 | 38.4 | 2.48-2.54 | m |
| 6-Me | 17.9 | 0.93 | d 6.68 |
| | (3H) | | |
| 7 | 79.2 | 4.47-4.54 | m |
| 7-Me | 18.8 | 0.85 | d 6.22 |
| | (3H) | | |
| Tyr Deriv. | | | |
| 1′ | 173.1 | x | |
| 2′ | 82.7 | 3.79 | d 10.21 |
| O—Me′ | 57.9 | 3.11 | s |
| | (3H) | | |
| 3′ | 55.2 | 4.96 | d 10.22 |
| 4′ | 135.6 | x | |
| 5′ | 130.3 | 7.14 | d 2.13 |
| 6′ | 123.9 | x | |
| 7′ | 153.3 | x | |
| 8′ | 117.3 | 7.04 | d 8.62 |
| 9′ | 127.2 | 6.85 | dd 8.63, 2.13 |
| N—Me-Trp | | | |
| 1″ | 170.4 | | |
| 2″ | 55.6 | 5.54 | dd 7.93, 7.93 |
| 3″ | 25.4 | 3.11; 2.87 | m; dd 13.95, 6.97 |
| | (2H) | | |
| 4″ | 105.9 | x | |
| 5″ | 122.9 | x | |
| 6″ | 128.2 | x | |
| 7″ | 135.8 | x | |
| 8″ | 111.6 | 7.51 | d 7.96 |
| 9″ | 122.5 | 7.06 | dd 7.44, 7.44 |
| 10″ | 120.1 | 6.99 | dd 7.70, 7.70 |
| 11″ | 118.6 | 7.51 | d 7.96 |
| N—Me″ | 30.8 | 3.17 | s |
| | (3H) | | |
| Ala | | | |
| 1′″ | 174.6 | x | |
| 2′″ | 45.6 | 4.82 | m, Waterpeak |
| 3′″ | 18.1 | 0.93 | d 6.94 |
| | (3H) | | |
| β-D-glucuronic | | | |
| acid | | | |
| 1 | 102.1 | 5.05 | d 7.70 |
| 2 | 74.3 | 3.62 | dd 9.04, 7.64 |
| 3 | 77.3 | 3.54 | dd 9.13, 9.13 |
| 4 | 72.9 | 3.65 | dd 9.19, 9.19 |
| 5 | 76.3 | 4 | d 9.72 |
| 6 | 172.9 | x | |

Chondramide E2 (700 MHz, 175 MHz, MeOD)
SBcm007

| | 13C | 1H | J (in Hertz) |
|---|---|---|---|
| PKS | | | |
| 1 | 176.5 | x | |
| 2 | 39.9 | 2.59-2.64 | m |
| 2-Me | 18.5 (3H) | 1.06 | d 6.70 |
| 3 | 46.1 (2H) | 2.19; | dd 12.98, 12.37 dd 13.65, |
| | | 2.04 | 3.83 |
| 4 | 134.3 | x | |
| 4-Me | 15.5 (3H) | 1.67 | s |
| 5 | 128.8 | 4.83 | Wasserpeak |
| 6 | 38.5 | 2.48-2.45 | m |
| 6-Me | 17.9 (3H) | 0.93 | d 6.72 |
| 7 | 78.7 | 4.41-4.49 | m |
| 7-Me | 18.6 (3H) | 0.84 | d 6.24 |
| Tyr Deriv. | | | |
| 1′ | 173.0 | x | |
| 2′ | 73.4 | 4.16 | d 10.23 |
| 3′ | 57.0 | 4.98 | dd 10.02, 9.87 |
| 4′ | 130.9 | x | |
| 5′ | 129.6 (2H) | 7.09 | d 8.63 |
| 6′ | 115.9 (2H) | 6.71 | d 8.63 |
| 7′ | 157.5 | x | |
| N—Me-Trp | | | |
| 1″ | 170.9 | x | |
| 2″ | 55.3 | 5.6 | dd 9.82, 6.36 |
| 3″ | 25.2 (2H) | 3.07; | dd 14.76, 9.78; dd14.76, |
| | | 2.90 | 6.36 |
| 4″ | 109.8 | x | |
| 5″ | 122.5 | x | |
| 6″ | 128.1 | x | |
| 7″ | 135.9 | x | |
| 8″ | 111.3 | 7.19 | d 8.06 |
| 9″ | 122.5 | 7.07 | ddd 8.18, 7.20, 1.07 |
| 10″ | 120.2 | 7.01 | ddd 7.98, 7.24, 0.90 |
| 11″ | 119.0 | 7.53 | d 7.84 |
| N—Me″ | 30.9 (3H) | 3.12 | s |
| Ala | | | |
| 1′″ | 174.5 | x | |
| 2′″ | 45.4 | 4.70-4.74 | m |
| 3′″ | 17.9 (3H) | 0.68 | d 7.03 |

Bromo Chondramide
A3 (700 MHz, 175 MHz, MeOD)
Cm7

| | 13C | 1H | J(in Hertz) |
|---|---|---|---|
| PKS | | | |
| 1 | 176.6 | x | |
| 2 | 39.9 | 2.64-2.70 | m |
| 2-Me | 18.6 | 1.09 | d 6.78 |
| | (3H) | | |
| 3 | 45.8 | 2.25; 2.05 | dd 13.41, 12.47; dd 13.23, 3.32 |
| | (2H) | | |
| 4 | 134.4 | x | |
| 4-Me | 15.7 | 1.7 | s |
| | (3H) | | |
| 5 | 128.8 | 4.84 | m, Waterpeak |
| 6 | 38.4 | 2.48-2.55 | m |
| 6-Me | 17.5 | 0.93 | d 6.74 |
| | (3H) | | |
| 7 | 78.0 | 4.51-4.55 | m |
| 7-Me | 18.4 | 0.88 | d 6.23 |
| | (3H) | | |
| Tyr Deriv. | | | |
| 1′ | 172.8 | x | |
| 2′ | 83.0 | 3.83 | d 9.96 |
| O—Me′ | 57.8 | 3.16 | s |
| | (3H) | | |
| 3′ | 55.1 | 4.99 | dd 9.65, 9.65 |
| 4′ | 132.9 | x | |
| 5′ | 132.8 | 7.33 | d 2.15 |
| 6′ | 110.1 | x | |
| 7′ | 154.3 | x | |
| 8′ | 116.7 | 6.75 | d 8.34 |
| 9′ | 128.3 | 6.9 | dd 8.38, 2.13 |
| N—Me-Trp | | | |
| 1″ | 170.9 | | |
| 2″ | 56.8 | 5.51 | dd 8.45, 7.63 |
| 3″ | 26.1 | 2.97-3.08 | m |
| | (2H) | | |
| 4″ | 109.6 | x | |
| 5″ | 124.7 | 6.85 | s |
| 6″ | 128.1 | x | |
| 7″ | 137.7 | x | |
| 8″ | 111.9 | 7.26 | d 8.10 |
| 9″ | 121.9 | 7.05 | dd 8.19, 6.88 |
| 10″ | 119.3 | 6.98 | dd 8.05, 6.96 |
| 11″ | 119.0 | 7.57 | d 7.87 |
| N—Me″ | 30.7 | 3.09 | s |
| | (3H) | | |
| Ala | | | |
| 1′″ | 174.4 | x | |
| 2′″ | 45.5 | 4.77-4.82 | m |
| 3′″ | 18.2 | 0.85 | d 6.96 |
| | (3H) | | |

Propionyl-Chondramide
C1 (700 MHz, 175 MHz, MeOD)
Cm7

| | 13C | 1H | J (in Hertz) |
|---|---|---|---|
| PKS | | | |
| 1 | 177.3 | x | |
| 2 | 39.8 | 2.65-2.70 | m |
| 2-Me | 19.4 (3H) | 1.12 | d 6.89 |
| 3 | 44.2 (2H) | 2.31; 2.01 | dd 14.53, 10.31; dd |
| | | | 14.53, 3.09 |
| 4 | 134.3 | x | |
| 4-Me | 16.7 (3H) | 1.63 | s |
| 5 | 129.3 | 4.96 | d 9.37 |
| 6 | 37.0 | 2.54-2.58 | m |
| 6-Me | 16.7 (3H) | 0.84 | d 6.92 |
| 7 | 81.6 | 4.57-4.61 | m |
| 7-Eth | 26.2 (2H) | 1.49-1.55; 1.32-1.39 | m; m |
| 7-Eth-Meth | 10.7 (3H) | 0.79 | t 7.45 |
| Tyr Deriv. | | | |
| 1′ | 172.5 | x | |
| 2′ | 42.3 (2H) | 2.72 | d 6.52 |
| 3′ | 51.2 | 5.18-5.22 | m |
| 4′ | 133.1 | x | |
| 5′ | 127.9 (2H) | 6.96 | d 7.47 |
| 6′ | 116.0 (2H) | 6.68 | d 8.60 |
| 7′ | 157.4 | x | |
| N—Me-Trp | | | |
| 1″ | 171.9 | | |
| 2″ | 57.4 | 5.54 | dd 9.30, 7.11 |
| 3″ | 25.0 (2H) | 3.23; 3.11 | dd 15.03, 7.09; dd |
| | | | 14.96, 9.38 |
| 4″ | 110.3 | x | |
| 5″ | 123.9 | 6.97 | s |
| 6″ | 128.3 | x | |
| 7″ | 137.7 | x | |
| 8″ | 112.0 | 7.33 | d 8.14 |
| 9″ | 122.2 | 7.09 | dd 8.08, 7.08 |
| 10″ | 119.5 | 7 | dd 8.01, 7.09 |
| 11″ | 119.0 | 7.58 | d 7.91 |
| N—Me″ | 30.2 (3H) | 3.05 | s |
| Ala | | | |
| 1′″ | 174.9 | x | |
| 2′″ | 46.2 | 4.76 | q 6.84 |
| 3′″ | 17.9 (3H) | 0.92 | d 6.88 |

Bromo-Chondramide
C3 (700 MHz, 175 MHz, MeOD)
Cm7

| | 13C | 1H | J (in Hertz) |
|---|---|---|---|
| PKS | | | |
| 1 | 176.6 | x | |
| 2 | 40.1 | 2.65-2.70 | m |
| 2-Me | 18.7 | 1.11 | d 6.80 |
| | (3H) | | |
| 3 | 46.0 | 2.27; 2.07 | dd 13.29, 12.25; dd 13.37, 3.15 |
| | (2H) | | |
| 4 | 134.6 | x | |
| 4-Me | 15.9 | 1.69 | s |
| | (3H) | | |
| 5 | 128.9 | 4.87 | m, Waterpeak |
| 6 | 38.1 | 2.50-2.56 | m |
| 6-Me | 17.5 | 0.91 | d 6.86 |
| | (3H) | | |
| 7 | 78.6 | 4.46-4.50 | m |
| 7-Me | 17.8 | 0.94 | d 6.26 |
| | (3H) | | |
| Tyr Deriv. | | | |
| 1′ | 172.6 | x | |
| 2′ | 42.5 | 2.57-2.65 | m, Waterpeak |
| | (2H) | | |
| 3′ | 51.2 | 5.28 | dd 11.53, 3.68 |
| 4′ | 135.1 | x | |
| 5′ | 131.4 | 7.33 | d 2.18 |
| 6′ | 110.5 | x | |
| 7′ | 154.4 | x | |
| 8′ | 117.0 | 6.76 | d 8.36 |
| 9′ | 127.1 | 6.87 | dd 8.04, 2.24 |
| N—Me-Trp | | | |
| 1″ | 170.9 | | |
| 2″ | 57.0 | 5.53 | dd 8.76, 7.51 |
| 3″ | 25.5 | 3.18; 3.08 | dd 14.68, 7.46; dd 14.93, 8.87 |
| | (2H) | | |
| 4″ | 109.9 | x | |
| 5″ | 123.9 | 6.94 | s |
| 6″ | 128.8 | x | |
| 7″ | 137.7 | x | |
| 8″ | 112.0 | 7.3 | d 8.00 |
| 9″ | 122.1 | 7.07 | dd 7.07, 6.07 |
| 10″ | 119.4 | 6.99 | dd 8.03, 7.07 |
| 11″ | 119.6 | 7.59 | d 7.93 |
| N—Me″ | 30.5 | 3.09 | s |
| | (3H) | | |
| Ala | | | |
| 1′″ | 174.4 | x | |
| 2′″ | 46.0 | 4.71-4.75 | m, Waterpeak |
| 3′″ | 17.9 | 0.88 | d 6.90 |
| | (3H) | | |

Propionyl-Bromo
Chondramide C3 (700 MHz, 175 MHz, MeOD)
Cm7

| | 13C | 1H | J (in Hertz) |
|---|---|---|---|
| PKS | | | |
| 1 | 177.2 | x | |
| 2 | 39.9 | 2.64-2.65 | m |
| 2-Me | 19.6 (3H) | 1.13 | d 6.94 |
| 3 | 44.2 (2H) | 2.32; 2.01 | dd 14.49, 10.76; dd |
| | | | 14.63, |
| 4 | 134.3 | x | 2.94 |
| 4-Me | 16.6 (3H) | 1.62 | s |
| 5 | 129.2 | 4.96 | d 9.45 |
| 6 | 36.8 | 2.54-2.58 | m |
| 6-Me | 16.5 (3H) | 0.84 | d 6.94 |
| 7 | 81.6 | 4.58-4.62 | m |
| 7-Eth | 26.3 (2H) | 1.49-1.55; 1.35-1.40 | m; m |
| 7-Eth-Meth | 10.6 (3H) | 0.78 | t 7.45 |
| Tyr Deriv. | | | |
| 1′ | 172.2 | x | |
| 2′ | 41.8 (2H) | 2.74 | d 6.36 |
| 3′ | 51.5 | 5.18 | t 6.22 |
| 4′ | 134.9 | x | |
| 5′ | 131.5 | 7.36 | d 2.23 |
| 6′ | 110.6 | x | |
| 7′ | 154.4 | x | |
| 8′ | 116.5 | 6.78 | d 8.38 |
| 9′ | 127.2 | 6.94 | dd 8.41, 2.22 |
| N—Me-Trp | | | |
| 1″ | 171.3 | | |
| 2″ | 57.5 | 5.53 | dd 9.47, 6.97 |
| 3″ | 24.8 (3H) | 3.15-3.24 | m |
| 4″ | 110.3 | x | |
| 5″ | 123.8 | 6.98 | s |
| 6″ | 128.2 | x | |
| 7″ | 137.7 | x | |
| 8″ | 111.9 | 7.32 | d 8.14 |
| 9″ | 122.1 | 7.07 | dd 8.23, 7.09 |
| 10″ | 119.4 | 6.99 | dd 7.98, 6.96 |
| 11″ | 119.3 | 7.57 | d 7.89 |
| N—Me″ | 31.20.2 (3H) | 3.04 | s |
| Ala | | | |
| 1′″ | 174.8 | x | |
| 2′″ | 46.2 | 4.71-4.75 | m |
| 3′″ | 18.0 (3H) | 0.88 | d 6.91 |

Chondramide A10
Variant (700 MHz, 175 MHz, MeOD)
Cm7

| | 13C | 1H | J (in Hertz) |
|---|---|---|---|
| PKS | | | |
| 1 | 176.3 | x | |
| 2 | 39.9 | 2.65-2.69 | m |
| 2-Me | 18.6 | 1.09 | d 6.70 |
| | (3H) | | |
| 3 | 45.9 | 2.23; 2.06 | dd 12.76, 12.76; dd 13.00, |
| | (2H) | | 3.13 |
| 4 | 134.3 | x | |
| 4-Me | 15.5 | 1.69 | s |
| | (3H) | | |
| 5 | 128.7 | 4.83 | m, Waterpeak |
| 6 | 38.4 | 2.48-2.54 | m |
| 6-Me | 17.7 | 0.93 | d 6.73 |
| | (3H) | | |
| 7 | 79.1 | 4.49-4.54 | m |
| 7-Me | 18.7 | 0.85 | d 6.25 |
| | (3H) | | |
| Tyr Deriv. | | | |
| 1′ | 172.7 | x | |
| 2′ | 82.6 | 3.82 | d 10.13 |
| O—Me′ | 57.8 | 3.12 | s |
| | (3H) | | |
| 3′ | 55.2 | 4.99 | dd 9.88, 9.87 |
| 4′ | 135.7 | x | |
| 5′ | 130.1 | 7.19 | d 2.30 |
| 6′ | 123.9 | x | |
| 7′ | 153.0 | x | |
| 8′ | 117.4 | 7.02 | d 8.61 |
| 9′ | 127.5 | 6.92 | dd 8.57, 2.05 |
| N—H | x | 8.55 | d 9.43 |
| N—Me-Trp | | | |
| 1″ | 170.3 | | |
| 2″ | 55.5 | 5.56 | dd 8.07, 8.07 |
| 3″ | 25.4 | 3.07, 2.92 | dd 14.44, 7.94 |
| | (2H) | | |
| 4″ | 105.9 | x | |
| 5″ | 122.8 | x | |
| 6″ | 127.9 | x | |
| 7″ | 135.7 | x | |
| 8″ | 111.5 | 7.17 | d 7.71 |
| 9″ | 122.6 | 7.07 | ddd 8.04, 7.15, 1.01 |
| 10″ | 120.1 | 6.99 | ddd 8.00, 7.21, 0.98 |
| 11″ | 118.9 | 7.5 | d 7.99 |
| N—Me″ | 30.9 | 3.15 | s |
| | (3H) | | |
| Ala | | | |
| 1′″ | 174.4 | x | |
| 2′″ | 45.6 | 4.81 | m, Waterpeak |
| 3′″ | 18.1 | 0.88 | d 6.97 |
| | (3H) | | |
| β-D-glucuronic | | | |
| acid | | | |
| 1 | 102.0 | 5.06 | d 7.70 |
| 2 | 74.5 | 3.63 | dd 9.15, 7.70 |
| 3 | 77.1 | 3.76 | dd 9.07, 9.07 |
| 4 | 78.7 | 3.92 | m 3.91-3.95 |
| 5 | 75.8 | 4.15 | d 4.15 |
| 6 | 171.1 | x | |
| 2-(acetylamino)-2-deoxy- | | | |
| 6-O-Methyl | | | |
| hexopyranose | | | |
| 1 | 99.5 | 5.35 | d 3.71 |
| 2 | 55.1 | 3.93 | m 3.91-3.95 |
| 3 | 72.7 | 3.61 | m 3.60-3.64 |
| 4 | 72.9 | 3.7 | m 3.68-3.71 |
| 5 | 71.4 | 3.49 | dd 9.75, 9.75 |
| 6 | 71.9 | 3.69; 3.60 | m 3.67-3.71; m 3.57-3.61 |
| | (2H) | | |
| 2,N-acetyl | 173.4 | X | |
| 2 | 22.5 | 2.02 | s |
| | (3H) | | |
| 6,O-Methyl | 59.4 | 3.39 | s |
| | (3H) | | |

The following compounds CA1 to CA9 have been prepared in analogy to the compounds described above:

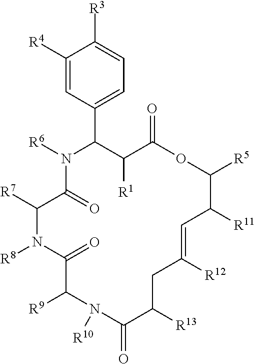

| | CA1 | CA2 | CA3 | CA4 | CA5 |
|---|---|---|---|---|---|
| R¹ | H | H | H | H | H |
| R³ | OH | OH | OH | OH | OH |
| R⁴ | Cl | Cl | Br | Cl | Cl |
| R⁵ | H | H | H | ethyl | ethyl |
| R⁶ | H | H | H | H | H |
| R⁷ | —CH₂-indole | —CH₂—Cl-indole | —CH₂-indole | isopropyl | isopropyl |
| R⁸ | methyl | methyl | methyl | methyl | methyl |
| R⁹ | methyl | methyl | methyl | methyl | methyl |
| R¹⁰ | H | H | H | H | H |
| R¹¹ | H | H | H | H | H |
| R¹² | methyl | methyl | methyl | methyl | methyl |
| R¹³ | H | H | H | H | H |
| m/z [M + H]⁺ | 609.2474 | 643.2085 | 653.1969 | 550.2678 | 550.2678 |
| theor. | | | | | |
| m/z [M + H]⁺ | 609.2474 | 643.2098 | 653.1951 | 550.2678 | 550.2692 |
| found | | | | | |

| | CA6 | CA7 | CA8 | CA9 |
|---|---|---|---|---|
| R¹ | H | H | H | H |
| R³ | OH | OH | OH | OH |
| R⁴ | Cl | Cl | Cl | Cl |
| R⁵ | ethyl | ethyl | ethyl | H |
| R⁶ | H | H | H | H |
| R⁷ | isopropyl | —CH₂—Cl-indole | —CH₂—Cl-indole | isopropyl |
| R⁸ | methyl | methyl | methyl | methyl |
| R⁹ | methyl | methyl | methyl | methyl |
| R¹⁰ | H | H | H | H |
| R¹¹ | H | H | H | H |
| R¹² | methyl | methyl | methyl | methyl |
| R¹³ | methyl | methyl | H | H |
| m/z [M + H]⁺ | 564.2835 | 685.2554 | 671.2398 | 522.2365 |
| theor. | | | | |
| m/z [M + H]⁺ | 564.2943 | 685.2558 | 671.2395 | 522.2337 |
| found | | | | |

These compounds were profiled regarding their growth inhibitory potential in a panel of cancer cell lines. Natural chondramide A was used as reference:

Biological evaluation of group A derivatives. GI₅₀ values [nM] were determined in a small panel of cancer cell lines.

| Compound | HCT-116 | KB-3.1 | RAW246.7 | U-2 OS |
|---|---|---|---|---|
| Chondramide A (ref.) | 91.7 | 71.6 | 47.3 | 68.5 |
| CA2 | 30.5 | 375.9 | 179.6 | 66.7 |
| CA7-Fr1 | 16.3 | 97.9 | 74.7 | 53.5 |
| CA1 | 1.3 | 4.8 | 6.1 | 2.8 |
| CA3 | 2.1 | 6.4 | 6.2 | 1.8 |
| CA7-Fr2 | 0.8 | 1.9 | 1.8 | 1.3 |
| CA8 | 0.2 | 1.1 | 0.8 | 0.6 |
| CA4 | 25.5 | 58.2 | 29.8 | >180 |
| CA5 | 77.3 | 68.7 | 56.2 | 153.3 |
| CA6-Fr1 | 79.4 | 71.4 | 41.1 | 59.6 |
| CA6-Fr2 | 100.7 | 74.5 | 104.9 | 65.4 |
| CA9 | 180.1 | >180 | >180 | >180 |

HCT-116: human colon carcinoma;
KB-3.1: human cervical carcinoma;
RAW246.7: murine leukemic monocyte macrophages;
U-2 OS: human osteosarcoma;
Fr: fraction

TABLE 1

| GI₅₀ values [nM] | HCT-116 | U-2 OS | KB-3.1 | KB-V.1 | L-929 | MRC5 | HUVEC |
|---|---|---|---|---|---|---|---|
| Chondramide A | 48.9 ± 14.6 | 27.1 ± 4.1 | 50.5 ± 15.4 | 38.0 ± 10.5 | 90.9 ± 11.8 | 63.8 ± 13.0 | 43.7 ± 7.0 |
| Chondramide B | 30.1 ± 9.5 | 20.8 ± 1.9 | 40.7 ± 9.4 | 56.4 ± 9.1 | 80.8 ± 13.6 | 20.9 ± 3.2 | 30.7 ± 2.9 |
| Chondramide C | 33.7 ± 3.2 | 25.6 ± 1.2 | 36.8 ± 6.8 | 85.8 ± 14.9 | 87.3 ± 14.9 | 26.1 ± 4.7 | 34.0 ± 3.7 |
| Chondramide A3 | 28.6 ± 3.9 | 46.7 ± 6.5 | 63.5 ± 12.6 | 85.7 ± 1.4 | 72.8 ± 3.6 | 38.0 ± 2.8 | 50.5 ± 5.6 |
| Chondramide A4 | 44.4 ± 1.6 | 26.4 ± 2.2 | 56.8 ± 7.0 | 83.1 ± 1.6 | 67.6 ± 9.8 | 12.7 ± 1.4 | 52.5 ± 7.4 |
| Chondramide A6 | 60.9 ± 3.5 | 51.6 ± 4.6 | 49.3 ± 7.6 | 23.1 ± 1.5 | 44.6 ± 5.5 | 25.9 ± 1.5 | 67.7 ± 10.4 |
| Chondramide A8 | 49.3 ± 7.6 | 33.2 ± 3.4 | 13.4 ± 3.0 | 58.1 ± 4.0 | 56.2 ± 12.5 | 7.6 ± 4.7 | 24.05 ± 7.9 |
| Chondramide A9 | n.a. | n.a. | n.a. | w.a. | w.a. | n.a. | n.a. |
| Chondramide A10 | n.a. | n.a. | w.a. | w.a. | n.a. | n.a. | w.a. |
| Bromo-Chondramide A3 | 42.9 ± 3.2 | 37.7 ± 2.8 | 74.5 ± 14.2 | 11.2 ± 0.4 | 69.4 ± 1.5 | 44.5 ± 4.9 | 32.7 ± 2.9 |
| Bromo-Chondramide C3 | 17.7 ± 1.3 | 4.8 ± 1.7 | 46.8 ± 6.5 | 23.8 ± 2.1 | 14.0 ± 1.4 | 57.7 ± 3.5 | 44.4 ± 7.0 |
| Propionyl-Chondramide C1 | 42.1 ± 3.2 | 15.1 ± 1.1 | 56.3 ± 9.6 | 22.7 ± 8.3 | 30.0 ± 7.7 | 71.9 ± 4.1 | 54.6 ± 5.5 |
| Propionyl-Bromo- | 17.2 ± 1.6 | 9.0 ± 0.6 | 38.0 ± 6.5 | 31.9 ± 2.0 | 23.0 ± 2.1 | 129.8 ± 3.7 | 50.2 ± 5.6 |
| Chondramide C3 | | | | | | | |
| Chondramide E4 | 378.8 ± 30.2 | 351.9 ± 30.6 | 627.2 ± 122.5 | 251.5 ± 79.1 | 554.8 ± 81.6 | 479.7 ± 63.6 | 207.4 ± 26.4 |

1. A compound of formula (I): 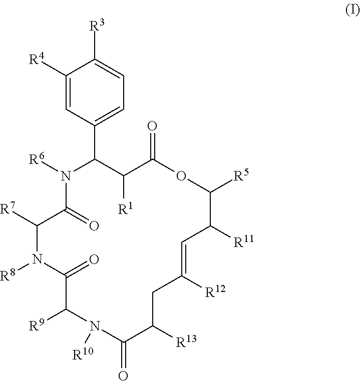
wherein
R¹ is a hydrogen atom, a hydroxy group, an alkyl, an alkenyl, or a heteroalkyl group;
R³ is a hydrogen atom, a halogen atom, a phosphate group, a hydroxy group, an amino group, a thiol group, an alkyl, an alkenyl, an alkynyl, a heteroalkyl, a cycloalkyl, a heterocycloalkyl, an alkylcycloalkyl, a heteroalkylcycloalkyl, an aryl, a heteroaryl, an aralkyl or a heteroaralkyl group;
R⁴ is a halogen atom or a hydrogen atom;
R⁵ is a hydrogen atom or an alkyl, an alkenyl or a heteroalkyl group;
R⁶ is a hydrogen atom or an alkyl group;
R⁷ is a hydrogen atom, an alkyl, an alkenyl, an alkynyl, a heteroalkyl, a cycloalkyl, a heterocycloalkyl, an alkylcycloalkyl, a heteroalkylcycloalkyl, an aryl, a heteroaryl, an aralkyl or a heteroaralkyl group;
R⁸ is a hydrogen atom or an alkyl group;
R⁹ is a hydrogen atom, an alkyl, an alkenyl, an alkynyl, a heteroalkyl, a cycloalkyl, a heterocycloalkyl, an alkylcycloalkyl, a heteroalkylcycloalkyl, an aryl, a heteroaryl, an aralkyl or a heteroaralkyl group;
R¹⁰ is a hydrogen atom or an alkyl group;
R¹¹ is a hydrogen atom or an alkyl group;
R¹² is a hydrogen atom or an alkyl group;
R¹³ is a hydrogen atom or an alkyl group; and/or
R⁷ and R⁸ and/or R⁹ and R¹⁰ together are part of an optionally substituted heterocycloalkyl group;
or a pharmaceutically acceptable salt, solvate or hydrate or a pharmaceutically acceptable formulation thereof.

2. A compound according to claim 1, wherein R⁴ is Br, F or Cl.

3. A compound according to claim 1, wherein R³ is a hydroxy group, a phosphate group, or one of the following groups:

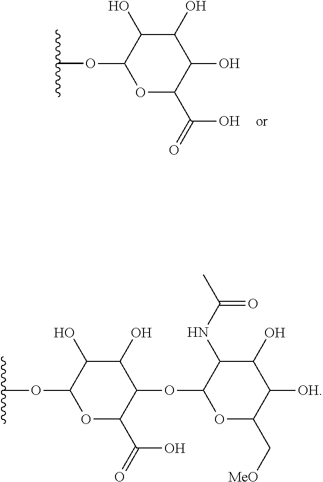 [SMILES: *OC1OC(C(=O)O)C(O)C(O)C1O ; "or" ; *OC1OC(C(=O)O)C(OC2OC(COC)C(O)C(O)C2NC(C)=O)C(O)C1O]

4. A compound according to claim 1, wherein R1 is a hydrogen atom, a hydroxy group or a methoxy group.

5. A compound according to claim 1, wherein R5 is a hydrogen atom or a C1-C8 alkyl group, preferably a C1-C8 alkyl group.

6. A compound according to claim 1, wherein R⁶ is a hydrogen atom or a methyl group.

7. A compound according to claim 1, wherein R⁷ is a C₁-C₆ alkyl group or a group of formula —CH₂-Ind, wherein Ind is an optionally substituted indole group.

8. A compound according to claim 1, wherein R⁷ has the following structure: 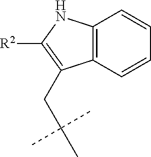
wherein R² a hydrogen atom or Cl; especially a hydrogen atom.

9. A compound according to claim 1, wherein R⁸ is a hydrogen atom or a methyl group.

10. A compound according to any one of the preceding claim 1, wherein R9 is a methyl group.

11. A compound according to claim, 1 wherein R10 is a hydrogen atom or a methyl group.

12. A compound according to claim 1, wherein R¹¹, R¹² and R¹³ are independently from each other a hydrogen atom or a methyl group.

13. A compound according to claim 1, wherein R7 and/or R9 is a group of formula —CH2-CH2-CH2-CH2-NH2 or —CH2-CH2-CH2-CH2-NHR71, wherein R71 is fluoresceinyl, diacetylated fluoresceinyl, rhodaminyl, Carboxytetramethylrhodaminyl (TAMRA), BODIPY or a cyanine.

14. A pharmaceutical composition comprising a compound according to claim 1 and optionally one or more carrier substances and/or one or more adjuvants.

15. (canceled)

16. A compound according to claim 1 wherein R3 is a hydroxy group.

17. A compound according to claim 1 wherein R1 is a hydrogen atom.

18. A compound according to claim 1 wherein R5 is an ethyl group.

19. A compound according to claim 1 wherein R6 is a hydrogen atom.

20. A method for treating a subject suffering from cancer, comprising administering to the subject and effective amount of a compound or pharmaceutical composition of claim 1.
